(19) United States
(12) Patent Application Publication
(10) Pub. No.: US 2014/0179678 A1
(43) Pub. Date: Jun. 26, 2014

(54) METHODS OF TARGETED TREATMENT OF FRONTOTEMPORAL LOBAR DEGENERATION

(71) Applicants: Holger Patzke, Belmont, MA (US); Gerhard Koenig, Newton, MA (US); Jean-Francois Blain, Jamaica Plain, MA (US)

(72) Inventors: Holger Patzke, Belmont, MA (US); Gerhard Koenig, Newton, MA (US); Jean-Francois Blain, Jamaica Plain, MA (US)

(21) Appl. No.: 14/007,572

(22) PCT Filed: Mar. 26, 2012
(86) PCT No.: PCT/US12/30527

§ 371 Date: Mar. 10, 2014

Related U.S. Application Data
(60) Provisional application No. 61/467,989, filed on Mar. 26, 2011.

(51) Int. Cl. C07D 267/20

(52) U.S. Cl. CPC C07D 267/20
USPC 514/211.11

(57) ABSTRACT

The present invention provides targeted treatment to subjects suffering from Frontotemporal lobar degeneration through use of FTLD targeted agents, as described in the present invention. In particular, the FTLD targeted agents provided herein demonstrate high brain penetration, which decreases risk issues associated with peripheral administration. Furthermore, the FTLD targeted agents of the present invention, when administered to a subject selected for treatment based on the results of a FTLD diagnostic assay, offer targeted treatment of FTLD.

### CROSS REFERENCE TO RELATED APPLICATIONS

This application claims the benefit of U.S. Provisional Application No. 61/467,989, filed on Mar. 26, 2011, which is incorporated herein by reference in its entirety.

### BACKGROUND

Frontotemporal lobar degeneration (FTLD) is a progressive neurodegenerative disorder representing about 5 percent of all dementia patients (Graff-Radford and Woodruff, Semin. Neurol., 27:48-57 (2007)). It is the second most common form of early-onset neurodegenerative dementia after Alzheimer's Disease (AD), affecting 10-20 percent of patients with an onset of dementia before 65 years. FTLD patients present with prominent behavioral and personality changes, often accompanied by language impairment, which evolve gradually into cognitive impairment and dementia (McKhann et al., Arch. Neurol., 58:1803-1809 (2001) and Neary et al., Neurology, 51:1546-54 (1998)). FTLD may occur alone or in combination with motor neuron disease (MND) (Lomen-Hoerth et al., Neurology, 59:1077-79 (2002)). The most common neuropathology associated with clinical FTLD is frontal and anterior temporal lobe atrophy with neuronal inclusions immunoreactive for ubiquitin and TAR-DNA binding protein 43 (TDP-43), but negative for tau and α-synuclein (FTLD-U) (Josephs et al., Neuropathol. Appl. Neurobiol., 30:369-73 (2004); Lipton et al., Acta. Neuropathol. (Berl), 108:379-85 (2004); and Mackenzie et al., Acta. Neuropathol., 112:551-59 (2006)). Neuronal cytoplasmic inclusions (NCIs) in the neocortex, striatum, and the dentate fascia of the hippocampus are the pathological hallmarks of FTLD-U. Up to four subtypes of FTLD-U have been delineated that are based on the distribution of NCIs, dystrophic neurites and the presence of neuronal intranuclear inclusions (NIIs). Almost all cases with PGRN mutations have a common FTLD-U subtype, characterized by NCIs, short thin neurites in layer II of the cortex and lentiform NIIs. This subtype is referred to as Type 1 by Mackenzie and coworkers (Mackenzie et al., Acta. Neuropathol., 112:539-49 (2006)) and Type 3 by Sampathu and co-workers (Sampathu et al., Am. J. Pathol., 169:1343-52 (2006)).

FTLD has a high familial incidence, with up to 50% of patients reported to have a family history of dementia. Recent molecular genetic advances in the field of FTLD have revealed that the genetic basis of FTLD-U is heterogeneous, and the causative mechanisms are just starting to be unraveled (Rademakers and Hutton, Curr. Neurol. Neurosci. Rep., 7:434-42 (2007)). Loss-of-function mutations in the gene encoding the secreted growth factor progranulin (PGRN) on chromosome 17 were identified as a major cause of familial FTLD-U, and are present in up to 25 percent of familial FTLD-U patients worldwide (Baker et al., Nature, 442:916-9 (2006); Cruts et al., Nature, 442:920-4 (2006); and Gass et al., Hum. Mol. Genet., 15:2988-3001 (2006)). In addition, mutations in the valosin containing protein gene (VCP) and the gene encoding the charged multivesicular body protein (CHMP2B) were reported in a small number of FTLD-U families (Skibinski et al., Nat. Genet., 37:806-8 (2005) and Watts et al., Nat. Genet., 36:377-81 (2004)).

With such low incidence of FTLD among dementia patients, there is significant need for treatment that is specific for this patient population and this specific disorder.

Frontotemporal dementia (FTD) is the clinical syndrome associated with FTLD. Symptoms can include: progressive inability to behave appropriately, empathize with others, learn, reason, make judgments, communicate and carry out daily activities.

### SUMMARY

The present invention provides targeted treatment to subjects suffering from FTD or FTLD (e.g, FTD associated with FTLD) through use of FTLD targeted agents, as described in the present invention. In particular, the FTLD targeted agents provided herein demonstrate high brain penetration, which decreases risk issues associated with peripheral administration. Furthermore, the FTLD targeted agents of the present invention, when administered to a subject selected for treatment based on the results of an FTD or FTLD diagnostic assay, offer targeted treatment of FTD or FTLD (e.g., patients suffering from FTD associated with FTLD).

Accordingly, in one aspect the invention provides a method for targeted treatment of Frontotemporal Dementia (FTD) or FrontoTemporal Lobar Degeneration (FTLD) in a subject. The method comprises administering an FTLD targeted agent to a subject identified as suffering from FTD or FTLD or both FTD and FTLD.

In another aspect, the invention provides a method for treating frontotemporal lobe dementia in a subject. The method comprises the step of administering an FTLD targeted agent to a subject identified as suffering from FTLD, such that the frontotemporal lobe dementia is treated in the subject.

In yet another aspect, the invention provides a diminished peripheral formulation comprising an FTLD targeted agent, and a pharmaceutically acceptable carrier, wherein the FTLD targeted agent is formulated to improve the targeted treatment of FTLD.

The invention provides a method of treating frontotemporal dementia (FTD) or frontotemporal lobar degeneration (FTLD) comprising administering to a patient in need thereof an effective amount of a composition comprising a compound of Formula (IV):

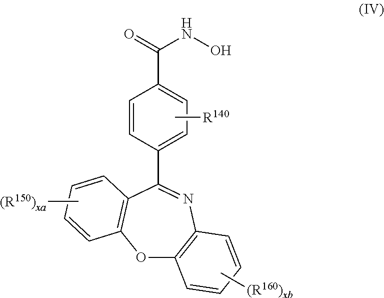

or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xa and xb denote numbers that are each independently selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiments the composition comprises a compound of Formula (V):

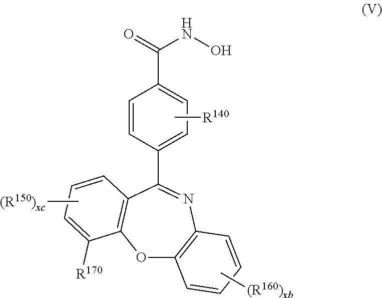

or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xb denotes a number selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xc is 0 or 1; and
R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —CO—C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl; and
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiment the compounds has formula V or a pharmaceutically acceptable salt thereof and xb and xc are 0.

In certain embodiment the compounds has formula V or a pharmaceutically acceptable salt thereof and R¹⁴⁰ is selected from the group consisting of: H, —OH, halo, —CN, —C₁-C₄alkyl, —C₁-C₄alkoxyl, —CF₃, —OCF₃, and —NO₂.

In certain embodiment the compounds has formula V or VI or a pharmaceutically acceptable salt thereof and R¹⁷⁰ is selected from: H, halo, —CN, —CF₃, —OCF₃, —C₁-C₆alkyl, and —C₁-C₆alkoxyl.

In certain embodiments the composition comprises a compound of Formula (VI):

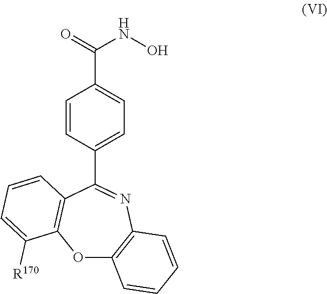

or a pharmaceutically acceptable salt thereof. In certain embodiments R¹⁷⁰ is selected from: H, halo, —CN, —CF₃, —OCF₃, —C₁-C₆alkyl, and —C₁-C₆alkoxyl.

In certain embodiments the composition comprises a compound selected from:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide,
(Z)—N-hydroxy-4-(5-(2-morpholinoethyl)-5H-dibenzo[b,e][1,4]diazepin-11-yl)benzamide,
   or a pharmaceutically acceptable salt thereof.

In some embodiments the composition comprises (Z)-4-(dibenzo[b,f][1,4]oxazepin-11-yl)-N-hydroxybenzamide or a pharmaceutically acceptable salt thereof.

In some embodiments the patient is suffering FTD.

In some embodiments the patient is suffering from FTLD.

In some embodiments the patient is suffering from FTD and FTLD.

The invention also includes a method of treating a patient at risk of developing frontotemporal dementia (FTD) or frontotemporal lobar degeneration (FTLD), comprising administering to the patient an effective amount of pharmaceutical composition comprising a compound of Formula (IV):

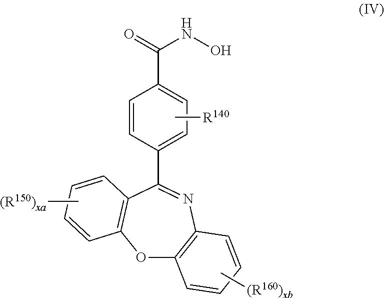

or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xa and xb denote numbers that are each independently selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiments the composition comprises a compound of Formula (V):

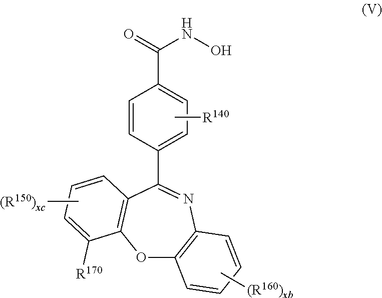

or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xb denotes a number selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xc is 0 or 1; and
R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —CO—C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl; and
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiment the compounds has formula V or a pharmaceutically acceptable salt thereof and xb and xc are 0.

In certain embodiment the compounds has formula V or a pharmaceutically acceptable salt thereof and R¹⁴⁰ is selected from the group consisting of: H, —OH, halo, —CN, —C₁-C₄alkyl, —C₁-C₄alkoxyl, —CF₃, —OCF₃, and —NO₂.

In certain embodiment the compounds has formula V or VI or a pharmaceutically acceptable salt thereof and R¹⁷⁰ is selected from: H, halo, —CN, —CF₃, —OCF₃, —C₁-C₆alkyl, and —C₁-C₆alkoxyl.

In certain embodiments the composition comprises a compound of Formula (VI):

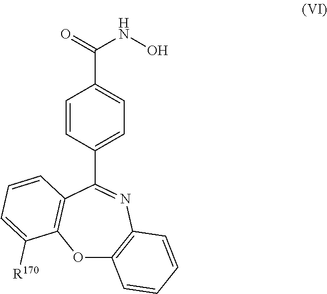

or a pharmaceutically acceptable salt thereof. In certain embodiments R¹⁷⁰ is selected from: H, halo, —CN, —CF₃, —OCF₃, —C₁-C₆alkyl, and —C₁-C₆alkoxyl.

In certain embodiments the composition comprises a compound selected from:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide,
(Z)—N-hydroxy-4-(5-(2-morpholinoethyl)-5H-dibenzo[b,e][1,4]diazepin-11-yl)benzamide,
   or a pharmaceutically acceptable salt thereof.

In some embodiments the composition comprises (Z)-4-(dibenzo[b,f][1,4]oxazepin-11-yl)-N-hydroxybenzamide or a pharmaceutically acceptable salt thereof.

In some embodiments of both the method of treating a patient suffering from FTD or FTLD or the patient at risk the patient harbors a mutant allele of the progranulin gene (e.g., a mutant T allele of rs5848).

In the forgoing methods: the compound is administered to a human patient at a daily oral dose of 10 mg-1 gm, 20-800 mg, 40-600 mg or 50-400 mg.

### DESCRIPTION OF THE DRAWINGS

FIG. 1 depicts the results of studies examining the effect of Compound 1 on the relative level of progranulin mRNA expression in primary cortical neurons derived from E17 Sprague-Dawley rats (0.1 and 0.3 μM Compound 1, FIG. 1A; 3.0 μM Compound 1, FIG. 1B).

FIG. 2 depicts the results of studies examining the effect of Compound 1 on progranulin mRNA (FIG. 2A) and protein (FIG. 2B) levels in FTLD patient lymphoblast cell lines.

FIG. 3 depicts the results of studies examining the effect of Compound 1 on progranulin mRNA (FIG. 3A) and protein (FIG. 3B) levels in primary fibroblasts from progranulin mutation carriers.

FIG. 4 depicts the results of studies examining the effect of Compound 1 on progranulin expression in immortalized lymphoblasts from a normal human subject. Bar graphs (FIG. 4B) represent the quantification of the western blot (FIG. 4A).

FIG. 5 depicts the results of studies examining the effect of Compound 1 on mice treated at 100 mg/kg Compound 1. FIG. 5A depicts progranulin mRNA relative expression in the cerebral cortex and FIG. 5B depicts progranulin protein expression in the cerebral cortex.

FIG. 6 depicts the results of studies examining the effect of Compound 1 on rats treated at 100 mg/kg Compound 1. FIG. 6A depicts CSF progranulin levels and FIG. 6B depicts plasma progranulin levels.

FIG. 7 depicts the results of studies examining the effect of Compound 1 on progranulin protein levels in rat primary cortical neurons.

### DETAILED DESCRIPTION

The present invention provides targeted treatment to subjects suffering from frontotemporal dementia or frontotemporal lobar degeneration through use of FTLD targeted agents, as described in the present invention. In particular, the FTLD targeted agents provided herein demonstrate high brain penetration, which decreases risk issues associated with peripheral administration. Furthermore, the FTLD targeted agents of the present invention, when administered to a subject selected for treatment based on the results of a FTD or FTLD diagnostic assay, offer targeted treatment of FTD or FTLD (e.g, FTD associated with FTLD).

The present invention, including targeted agents, methods, and pharmaceutical compositions will be described with reference to the following definitions that, for convenience, are set forth below. Unless otherwise specified, the below terms used herein are defined as follows:

### I. DEFINITIONS

For purposes of the present disclosure, the following definitions will be used (unless expressly stated otherwise).

The terms “treating”, “treatment”, or the like, as used herein covers the treatment of a disease-state in an animal and includes at least one of: (i) preventing the disease-state from occurring, in particular, when such animal is predisposed to the disease-state but has not yet developed symptoms of having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, preferably eliminating or curing of the disease. In a preferred embodiment the terms “treating”, “treatment”, or the like, covers the treatment of a disease-state in an animal and includes at least one of (ii), (iii) and (iv) above. In a preferred embodiment of the present disclosure the animal is a mammal, preferably a primate, more preferably a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

The expressions “targeted treatment of FrontoTemporal Lobar Degeneration” and targeted treatment of FTLD” are used interchangeably herein, and describe a method of treatment that offers a high level of success in treating subjects with FTLD or frontotemporal lobe dementia, as measured clinically and/or quantitatively through progranulin or progranulin mRNA levels. Such targeted treatment is based on the understanding described herein that there is significant correlation, e.g., greater than 80%, e.g., greater than 85%, e.g., greater than 90%, e.g., greater than 91%, e.g., greater than 92%, e.g., greater than 93%, e.g., greater than 94% e.g., greater than 95%, e.g., greater than 96%, e.g., greater than 97%, e.g., greater than 98%, e.g., greater than 99%, e.g., greater than 99.5%, e.g., 100%, between the incidence of mutations of the progranulin gene that effect progranulin levels and FTLD. The compounds of the invention, i.e., the “FTLD targeted agents” operate to restore or increase progranulin expression. In particular embodiments, the FTLD targeted agent has an acceptable safety profile, where blood plasma levels are sufficiently safe and afford brain penetration at doses that achieve the desired effect, e.g., FTLD targeted treatment

As used herein, the terms “histone deacetylase” and “HDAC” are intended to refer to any one of a family of enzymes that remove acetyl groups from a protein, such as for example, the F-amino groups of lysine residues at the N-terminus of a histone. Unless otherwise indicated by context, the term “histone” is meant to refer to any histone protein, including H1, H2A, H2B, H3, H4, and H5, from any species. Preferred histone deacetylases include class I and class II enzymes. Other preferred histone deacetylases include class IV enzymes. Preferably the histone deacetylase is a human HDAC, including, but not limited to, HDAC-1, HDAC-2, HDAC-3, HDAC-4, HDAC-5, HDAC-6, HDAC-7, HDAC-8, HDAC-9, HDAC-10 and HDAC-11. In some other preferred embodiments, the histone deacetylase is derived from a protozoal or fungal source.

The terms “histone deacetylase inhibitor” and “inhibitor of histone deacetylase” are intended to mean a compound having a structure as defined herein, which is capable of interacting with a histone deacetylase and inhibiting its enzymatic activity.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” is intended to mean a straight or branched chain aliphatic group having from 1 to 12 carbon atoms, preferably 1-8 carbon atoms, and more preferably 1-6 carbon atoms. Other preferred alkyl groups have from 2 to 12 carbon atoms, preferably 2-8 carbon atoms and more preferably 2-6 carbon atoms. Preferred alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms, preferably 2-8 carbon atoms, and more preferably 2-6 carbon atoms. Preferred alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms, preferably 2-8 carbon atoms, and more preferably 2-6 carbon atoms. Preferred alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” is intended to mean a saturated or unsaturated mono-, bi, tri- or poly-cyclic hydrocarbon group having about 3 to 15 carbons, preferably having 3 to 12 carbons, preferably 3 to 8 carbons, and more preferably 3 to 6 carbons. In certain preferred embodiments, the cycloalkyl group is fused to an aryl, heteroaryl or heterocyclic group. Preferred cycloalkyl groups include, without limitation, cyclopenten-2-enone, cyclopenten-2-enol, cyclohex-2-enone, cyclohex-2-enol, cyclopropyl, cyclobutyl, cyclopentyl, cyclopentenyl, cyclohexyl, cyclohexenyl, cycloheptyl, and cyclooctyl.

In certain preferred embodiments, the cycloalkyl group is a bridged cycloalkyl group, preferably a C₅-C₁₀ bridged bicyclic group. In certain preferred embodiments, the bridged cycloalkyl group is a C₅ bridged bicyclic group. In certain preferred embodiments, the bridged cycloalkyl group is a C₆ bridged bicyclic group. In certain preferred embodiments, the bridged cycloalkyl group is a C₇ bridged bicyclic group. In certain preferred embodiments, the bridged cycloalkyl group is a C₈ bridged bicyclic group. In certain preferred embodiments, the bridged cycloalkyl group is a C₉ bridged bicyclic. In certain preferred embodiments, the bridged cycloalkyl group has a bridge of 0, 1, 2 or 3 carbon atoms. A bridge of 0 carbon atoms is a bond, and equates to a cycloalkyl group fused to another ring structure. In certain preferred embodiments, the bridged cycloalkyl group has a bridge of 0, 1 or 3 carbon atoms. In certain preferred embodiments, the bridged cycloalkyl group has a bridge of 1 or 3 carbon atoms. In certain preferred embodiments, the bridged cycloalkyl group has a bridge of 1 carbon atom. In certain preferred embodiments, the bridged cycloalkyl group has a bridge of 2 carbon atoms. In certain preferred embodiments, the bridged cycloalkyl group has a bridge of 3 carbon atoms. If a bridged cycloalkyl group is described as “optionally substituted”, it is intended to be optionally substituted on any position, including the bridge. The bridged cycloalkyl group is not limited to any particular stereochemistry.

The term “heteroalkyl” is intended to mean a saturated or unsaturated, straight or branched chain aliphatic group, wherein one or more carbon atoms in the chain are independently replaced by a heteroatom selected from the group consisting of O, S(O)₀₋₂, N and N(R³³).

The term “aryl” is intended to mean a mono-, bi-, tri- or polycyclic C₆-C₁₄ aromatic moiety, preferably comprising one to three aromatic rings. Preferably, the aryl group is a C₆-C₁₀ aryl group, more preferably a C₆ aryl group. Preferred aryl groups include, without limitation, phenyl, naphthyl, anthracenyl, and fluorenyl.

The terms “aralkyl” or “arylalkyl” is intended to mean a group comprising an aryl group covalently linked to an alkyl group. If an aralkyl group is described as “optionally substituted”, it is intended that either or both of the aryl and alkyl moieties may independently be optionally substituted or unsubstituted. Preferably, the aralkyl group is (C₁-C₆)alkyl(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain preferred embodiments, the heterocyclic group is non-aromatic. In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a bridged heterocyclic group, preferably a C₆-C₁₀ bridged bicyclic group, wherein one or more carbon atoms are independently replaced by a heteroatom selected from the group consisting of N, O and S. In certain preferred embodiments, the bridged heterocyclic group is a C₆ bridged bicyclic group. In certain preferred embodiments, the bridged heterocyclic group is a C₇ bridged bicyclic group. In certain preferred embodiments, the bridged heterocyclic group is a C₈ bridged bicyclic group. In certain preferred embodiments, the bridged heterocyclic group is a C₉ bridged bicyclic. In certain preferred embodiments, the bridged heterocyclic group has a bridge of 0, 1, 2 or 3 carbon atoms. In certain preferred embodiments, the bridged heterocyclic group has a bridge of 0, 1 or 3 carbon atoms. A bridge of 0 carbon atoms is a bond, and equates to a heterocyclic group fused to another ring structure. In certain preferred embodiments, the bridged heterocyclic group has a bridge of 1 or 3 carbon atoms. In certain preferred embodiments, the bridged heterocyclic group has a bridge of 1 carbon atom. In certain preferred embodiments, the bridged heterocyclic group has a bridge of 2 carbon atoms. In certain preferred embodiments, the bridged heterocyclic group has a bridge of 3 carbon atoms. If a bridged heterocyclic group is described as “optionally substituted”, it is intended to be optionally substituted on any position, including the bridge. The bridged heterocyclic group is not limited to any particular stereochemistry.

In certain preferred embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 μl electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thianthrenyl, thiazolyl, thienyl, thienothiazolyl, thienooxazolyl, thienoimidazolyl, thiophenyl, triazinyl, triazolyl (e.g., 1,2,3-triazolyl, 1,2,4-triazolyl, 1,2,5-triazolyl, 1,3,4-triazolyl), and xanthenyl.

Aromatic polycycles include, but are not limited to, bicyclic and tricyclic fused ring systems, including for example naphthyl.

Non-aromatic polycycles include, but are not limited to, bicyclic and tricyclic fused ring systems where each ring can be 4-9 membered and each ring can containing zero, 1 or more double and/or triple bonds. Suitable examples of non-aromatic polycycles include, but are not limited to, decalin, octahydroindene, perhydrobenzocycloheptene and perhydrobenzo-[f]-azulene.

Polyheteroaryl groups include bicyclic and tricyclic fused rings systems where each ring can independently be 5 or 6 membered and contain one or more heteroatom, for example, 1, 2, 3 or 4 heteroatoms, independently chosen from O, N and S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline, and the like.

Non-aromatic polyheterocyclic groups include but are not limited to bicyclic and tricyclic ring systems where each ring can be 4-9 membered, contain one or more heteroatom, for example 1, 2, 3 or 4 heteroatoms, independently chosen from O, N and S, and contain zero, or one or more C—C double or triple bonds. Suitable examples of non-aromatic polyheterocycles include but are not limited to, hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydrop-1H-dicyclopenta[b,e]pyran.

Mixed aryl and non-aryl polyheterocycle groups include but are not limited to bicyclic and tricyclic fused ring systems where each ring can be 4-9 membered, contain one or more heteroatom independently chosen from O, N and S and at least one of the rings must be aromatic. Suitable examples of mixed aryl and non-aryl polyheteorcycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydropyrido[2,3-b][1,4]diazepin-4-one, 1,2,3,4,6,11-hexahydro-benzo[b]pyrido[2,3-e][1,4]diazepine-5-one, methylenedioxyphenyl, bis-methylenedioxyphenyl, 1,2,3,4-tetrahydronaphthalene, dibenzosuberane dihydroanthracene and 9H-fluorene.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and
(c) —(CR³²R^{33a})ₛ—NR³⁰R³¹, wherein s is from 0 (in which case the nitrogen is directly bonded to the moiety that is substituted) to 6, R³² and R^{33a} are each independently hydrogen, halo, hydroxyl or C₁-C₄alkyl, and R³⁰ and R³¹ are each independently hydrogen, cyano, oxo, hydroxyl, —C₁-C₈ alkyl, C₁-C₈ heteroalkyl, C₁-C₈ alkenyl, carboxamido, C₁-C₃ alkyl-carboxamido, carboxamido-C₁-C₃ alkyl, amidino, C₂-C₈hydroxyalkyl, C₁-C₃ alkylaryl, aryl-C₁-C₃ alkyl, C₁-C₃ alkylheteroaryl, heteroaryl-C₁-C₃ alkyl, C₁-C₃ alkylheterocyclyl, heterocyclyl-C₁-C₃ alkyl C₁-C₃ alkylcycloalkyl, cycloalkyl-C₁-C₃ alkyl, C₂-C₈ alkoxy, C₂-C₈ alkoxy-C₁-C₄alkyl, C₁-C₈ alkoxycarbonyl, aryloxycarbonyl, aryl-C₁-C₃ alkoxycarbonyl, heteroaryloxycarbonyl, heteroaryl-C₁-C₃ alkoxycarbonyl, C₁-C₈ acyl, C₀-C₈ alkyl-carbonyl, aryl-C₀-C₈ alkyl-carbonyl, heteroaryl-C₀-C₈ alkyl-carbonyl, cycloalkyl-C₀-C₈ alkyl-carbonyl, C₀-C₈ alkyl-NH-carbonyl, aryl-C₀-C₈ alkyl-NH-carbonyl, heteroaryl-C₀-C₈ alkyl-NH-carbonyl, cycloalkyl-C₀-C₈ alkyl-NH-carbonyl, C₀-C₈ alkyl-O-carbonyl, aryl-C₀-C₈ alkyl-O-carbonyl, heteroaryl-C₀-C₈ alkyl-O-carbonyl, cycloalkyl-C₀-C₈ alkyl-O-carbonyl, C₁-C₈ alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃ alkyl-, cycloalkyl-C₁-C₃ alkyl-, heterocyclyl-C₁-C₃ alkyl-, heteroaryl-C₁-C₃ alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, CO—C₃alkyl-S(O)₀₋₂—CO—C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocycyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰), —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl —CO—.

When there are two optional substituents bonded to adjacent atoms of a ring structure, such as for example phenyl, thiophenyl, or pyridinyl, the substituents, together with the atoms to which they are bonded, optionally form a 5- or 6-membered cycloalkyl or heterocycle having 1, 2, or 3 annular heteroatoms.

In a preferred embodiment, hydrocarbyl, alkyl, alkenyl, alkynyl, heteroalkyl, cycloalkyl, heterocyclic, aryl, heteroaryl, aromatic polycycle, non-aromatic polycycle, polyheteroaryl, non-aromatic polyheterocyclic and mixed aryl and non-aryl polyheterocycle groups are unsubstituted.

In other preferred embodiments, hydrocarbyl, alkyl, alkenyl, alkynyl, heteroalkyl, cycloalkyl, heterocyclic, aryl, heteroaryl, aromatic polycycle, non-aromatic polycycle, polyheteroaryl, non-aromatic polyheterocyclic and mixed aryl and non-aryl polyheterocycle groups are substituted with from 1 to 3 independently selected substituents.

Preferred substituents on alkyl groups include, but are not limited to, hydroxyl, halogen (e.g., a single halogen substituent or multiple halo substituents; in the latter case, groups such as CF₃ or an alkyl group bearing more than one Cl), cyano, nitro, alkyl, cycloalkyl, alkenyl, cycloalkenyl, alkynyl, heterocycle, aryl, —OR^{u}, —SR^{u}, —S(═O)R^{y}, —S(═O)₂R^{y}, —P(═O)₂R^{y}, —S(═O)₂OR^{y}, —P(═O)₂OR^{y}, —NR^{v}R^{w}, —NR^{v}S(═O)₂R^{y}, —NR^{v}P(═O)₂R^{y}, —S(═O)₂NR^{v}R^{w}, —P(═O)₂NR^{v}R^{w}, —C(═O)OR^{y}, —C(═O)R^{u}, —C(═O)NR^{v}R^{w}, —OC(═O)R^{u}, —OC(═O)NR^{v}R^{w}, —NR^{v}C(═O)OR^{y}, —NR^{x}C(═O)NR^{v}R^{w}, —NR^{x}S(═O)₂NR^{v}R^{w}, —NR^{x}P(═O)₂NR^{v}R^{w}, —NR^{v}C(═O)R^{u} or —NR^{v}P(═O)₂R^{y}, wherein R^{u} is hydrogen, alkyl, cycloalkyl, alkenyl, cycloalkenyl, alkynyl, heterocycle or aryl; R^{v}, R^{w} and R^{x} are independently hydrogen, alkyl, cycloalkyl, heterocycle or aryl, or said R^{v} and R^{w} together with the N to which they are bonded optionally form a heterocycle; and R^{y} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Preferred substituents on alkenyl and alkynyl groups include, but are not limited to, alkyl or substituted alkyl, as well as those groups recited as preferred alkyl substituents.

Preferred substituents on cycloalkyl groups include, but are not limited to, nitro, cyano, alkyl or substituted alkyl, as well as those groups recited about as preferred alkyl substituents. Other preferred substituents include, but are not limited to, spiro-attached or fused cyclic substituents, preferably spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on cycloalkenyl groups include, but are not limited to, nitro, cyano, alkyl or substituted alkyl, as well as those groups recited as preferred alkyl substituents. Other preferred substituents include, but are not limited to, spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Preferred substituents on aryl groups include, but are not limited to, nitro, cycloalkyl or substituted cycloalkyl, cycloalkenyl or substituted cycloalkenyl, cyano, alkyl or substituted alkyl, as well as those groups recited above as preferred alkyl substituents. Other preferred substituents include, but are not limited to, fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted. Still other preferred substituents on aryl groups (phenyl, as a non-limiting example) include, but are not limited to, haloalkyl and those groups recited as preferred alkyl substituents.

Preferred substituents on heterocylic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as preferred alkyl substituents. Other preferred substituents on heterocyclic groups include, but are not limited to, spiro-attached or fused cyclic substituents at any available point or points of attachment, more preferably spiro-attached cycloalkyl, spiro-attached cycloalkenyl, spiro-attached heterocycle (excluding heteroaryl), fused cycloalkyl, fused cycloakenyl, fused heterocycle and fused aryl, where the aforementioned cycloalkyl, cycloalkenyl, heterocycle and aryl substituents can themselves be optionally substituted.

In a preferred embodiment, a heterocyclic group is substituted on carbon, nitrogen and/or sulfur at one or more positions. Preferred substituents on nitrogen include, but are not limited to N-oxide, alkyl, aryl, aralkyl, alkylcarbonyl, alkylsulfonyl, arylcarbonyl, arylsulfonyl, alkoxycarbonyl, or aralkoxycarbonyl. Preferred substituents on sulfur include, but are not limited to, oxo and C₁₋₆alkyl. In certain preferred embodiments, nitrogen and sulfur heteroatoms may independently be optionally oxidized and nitrogen heteroatoms may independently be optionally quaternized.

Especially preferred substituents on alkyl groups include halogen and hydroxy.

Especially preferred substituents on ring groups, such as aryl, heteroaryl, cycloalkyl and heterocyclyl, include halogen, alkoxy and alkyl.

Preferred substituents on aromatic polycycles include, but are not limited to, oxo, C₁-C₆alkyl, cycloalkylalkyl (e.g. cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR^{aa}, such as alkoxy, wherein R^{aa} is selected from the group consisting of H, C₁-C₆alkyl, C₄-C₉cycloalkyl, C₄-C₉heterocycloalkyl, aryl, heteroaryl, arylalkyl, heteroarylalkyl and (CH₂)₀₋₆Z^{a}R^{bb}, wherein Z^{a} is selected from the group consisting of O, NR^{cc}, S and S(O), and R^{bb} is selected from the group consisting of H, C₁-C₆alkyl, C₄-C₉cycloalkyl, C₄-C₉heterocycloalkyl, C₄-C₉heterocycloalkylalkyl, aryl, mixed aryl and non-aryl polycycle, heteroaryl, arylalkyl, (e.g. benzyl), and heteroarylalkyl (e.g. pyridylmethyl); and R^{cc} is selected from the group consisting of H, C₁-C₆alkyl, C₄-C₉cycloalkyl, C₄-C₉heterocycloalkyl, aryl, heteroaryl, arylalkyl (e.g. benzyl), heteroarylalkyl (e.g. pyridylmethyl) and amino acyl.

Preferred substituents on non-aromatic polycycles include, but are not limited to, oxo, C₃-C₉cycloalkyl, such as cyclopropyl, cyclobutyl, cyclopentyl, cyclohexyl and the like. Unless otherwise noted, non-aromatic polycycle substituents include both unsubstituted cycloalkyl groups and cycloalkyl groups that are substituted by one or more suitable substituents, including but not limited to, C₁-C₆alkyl, oxo, halo, hydroxy, aminoalkyl, oxyalkyl, alkylamino and OR^{aa}, such as alkoxy. Preferred substituents for such cycloalkyl groups include halo, hydroxy, alkoxy, oxyalkyl, alkylamino and aminoalkyl.

Preferred substituents on carbon atoms of polyheteroaryl groups include but are not limited to, straight and branched optionally substituted C₁-C₆alkyl, unsaturation (i.e., there are one or more double or triple C—C bonds), acyl, oxo, cycloalkyl, halo, oxyalkyl, alkylamino, aminoalkyl, acylamino, OR^{aa} (for example alkoxy), and a substituent of the formula —O—(CH₂CH═CH(CH₃)(CH₂))₁₋₃H. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Preferably substitutions on nitrogen atoms include, for example by N-oxide or R^{cc}. Preferred substituents on nitrogen atoms include H, C₁-C₄alkyl, acyl, aminoacyl and sulfonyl. Preferably sulfur atoms are unsubstituted. Preferred substituents on sulfur atoms include but are not limited to oxo and lower alkyl.

Preferred substituents on carbon atoms of non-aromatic polyheterocyclic groups include but are not limited to straight and branched optionally substituted C₁-C₆alkyl, unsaturation (i.e., there are one or more double or triple C—C bonds), acyl, oxo, cycloalkyl, halo, oxyalkyl, alkylamino, aminoalkyl, acylamino and OR^{aa}, for example alkoxy. Examples of suitable straight and branched C₁-C₆alkyl substituents include but are not limited to methyl, ethyl, n-propyl, 2-propyl, n-butyl, sec-butyl, t-butyl and the like. Preferred substituents include halo, hydroxy, alkoxy, oxyalkyl, alkylamino and aminoalkyl. Preferably substitutions on nitrogen atoms include, for example, N-oxide or R^{cc}. Preferred N substituents include H, C₁-C₄ alkyl, acyl, aminoacyl and sulfonyl. Preferably, sulfur atoms are unsubstituted. Preferred S substituents include oxo and lower alkyl.

Preferred substituents on mixed aryl and non-aryl polyheterocycle groups include, but are not limited to, nitro or as described above for non-aromatic polycycle groups. Preferred substituents on carbon atoms include, but are not limited to, —N—OH, ═N—OH, optionally substituted alkyl, unsaturation (i.e., there are one or more double or triple C—C bonds), oxo, acyl, cycloalkyl, halo, oxyalkyl, alkylamino, aminoalkyl, acylamino and OR^{aa}, for example alkoxy. Preferably substitutions on nitrogen atoms include, for example, N-oxide or R^{cc}. Preferred N substituents include H, C₁₋₄alkyl, acyl aminoacyl and sulfonyl. Preferably sulfur atoms are unsubstituted. Preferred S substituents include oxo and lower alkyl.

A “halohydrocarbyl” is a hydrocarbyl moiety in which from one to all hydrogens have been replaced with one or more halo.

The term “halogen” or “halo” is intended to mean chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

The term “radical” is intended to mean a chemical moiety comprising one or more unpaired electrons.

Where optional substituents are chosen from “one or more” groups it is to be understood that this definition includes all substituents being chosen from one of the specified groups or the substituents being chosen from two or more of the specified groups.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

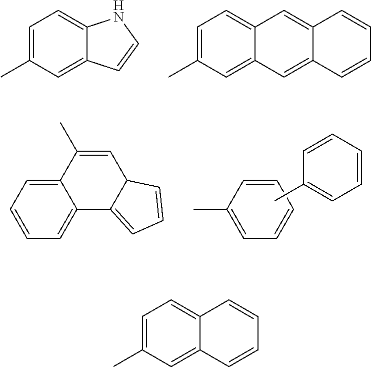

An “unsubstituted” moiety (e.g., unsubstituted cycloalkyl, unsubstituted heteroaryl, etc.) means that moiety as defined above that does not have an optional substituent. Thus, for example, “unsubstituted aryl” does not include phenyl substituted with a halo.

The term “protecting group” is intended to mean a group used in synthesis to temporarily mask the characteristic chemistry of a functional group because it interferes with another reaction. A good protecting group should be easy to put on, easy to remove and in high yielding reactions, and inert to the conditions of the reaction required. A protecting group or protective group is introduced into a molecule by chemical modification of a functional group in order to obtain chemoselectivity in a subsequent chemical reaction. One skilled in the art will recognize that during any of the processes for preparation of the compounds in the present disclosure, it may be necessary and/or desirable to protect sensitive or reactive groups on any of the molecules concerned. This may be achieved by means of conventional protecting groups, such as but not limited to Bn- (or —CH₂Ph), —CHPh₂, alloc (or CH₂═CH—CH₂—O—C(O)—), BOC-, -Cbz (or Z—), —F-moc, —C(O)—CF₃, N-Phthalimide, 1-Adoc-, TBDMS-, TBDPS-, TMS-, TIPS-, IPDMS-, —SiR₃, SEM-, t-Bu-, Tr-, THP- and Allyl-. These protecting groups may be removed at a convenient stage using methods known from the art.

The term “therapeutically effective amount” as that term is used herein refers to an amount which elicits the desired therapeutic effect. The therapeutic effect is dependent upon the disease being treated and the results desired. As such, the therapeutic effect can be a decrease in the severity of symptoms associated with the disease and/or inhibition (partial or complete) of progression of the disease. Further, the therapeutic effect can be the increase in production of progranulin in the brain. The amount needed to elicit the therapeutic response can be determined based on the age, health, size and sex of the patient. Optimal amounts can also be determined based on monitoring of the patient's response to treatment. Administration may be by any route, including, without limitation, parenteral, oral, sublingual, transdermal, topical, intranasal, intratracheal, or intrarectal. In certain particularly preferred embodiments, compounds of the disclosure are administered intravenously in a hospital setting. In certain other preferred embodiments, administration may preferably be by the oral route.

Some compounds of the disclosure may have one or more chiral centers and/or geometric isomeric centers (E- and Z-isomers), and it is to be understood that the disclosure encompasses all such optical, diastereoisomers and geometric isomers. The disclosure also comprises all tautomeric forms of the compounds disclosed herein.

The present disclosure also includes prodrugs of compounds of the disclosure. The term “prodrug” is intended to represent covalently bonded carriers, which are capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the disclosure include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like.

The compounds of the disclosure may be administered as is or as a prodrug, for example in the form of an in vivo hydrolyzable ester or in vivo hydrolyzable amide. An in vivo hydrolyzable ester of a compound of the disclosure containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any appropriate carboxy group in the compounds of this disclosure.

An in vivo hydrolyzable ester of a compound of the disclosure containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the disclosure containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

For simplicity, and unless stated otherwise, a moiety is written in the direction corresponding to the order given, for example, in Formula (I). For example, if J is —C₀₋₆alkyl-aryl-C₂₋₆heteroalkyl-, it is meant that the —C₀₋₆alkyl- portion is attached to Q and the —C₂₋₆heteroalkyl- portion is attached to L.

### II. COMPOUNDS OF THE INVENTION

The present invention provides targeted treatment to subjects suffering from FTD or FTLD through the use of FTLD targeted agents, as described in the present invention. In particular, the FTLD targeted agents provided herein demonstrate high brain penetration, which decreases risk issues associated with peripheral administration. Furthermore, the FTLD targeted agents of the present invention, when administered to a subject selected for treatment based on the results of a FTD or FTLD diagnostic assay, offer targeted treatment of FTD or FTLD. Thus, the compounds are useful for treating a subject suffering from FTD associated with FTLD and for treating a subject suffering from FTLD associated with reduced expression of progranulin before or after exhibiting symptoms of FTD.

The compounds of the invention described herein have been identified as HDAC inhibitors with unexpectedly enhanced utility as FTLD targeted agents due to increased brain penetration, and hence a safer therapeutic profile. As such, the compounds of the invention may be used to provide targeted treatment to subjects suffering from Frontotemporal lobar degeneration.

In a first embodiment, the FTLD targeted agents are represented by Formula (I):

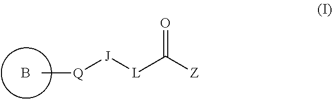

and N-oxides, hydrates, solvates, pharmaceutically acceptable salts, prodrugs and complexes thereof, and racemic mixtures, diastereomers and enantiomers thereof, wherein

Z is selected from the group consisting of —N(R¹)OR² and H;

L is selected from the group consisting of a covalent bond and —N(OR²)—;

wherein, when L is —N(OR²)—, Z is H; and

wherein, when Z is H, L is —N(OR²)—;

J is selected from the group consisting of a covalent bond, ═CH—, —C₁-C₈alkyl-, —C₀-C₃alkyl-C₁-C₈heteroalkyl-C₀-C₃alkyl-, —C₀-C₃alkyl-C₂-C₈alkenyl-C₀-C₃alkyl-, —C₀-C₃alkyl-C₂-C₈alkynyl-C₀-C₃alkyl-, —C₀-C₆alkyl-aryl-C₀-C₆alkyl-, —C₀-C₆alkyl-aryl-C₂-C₆heteroalkyl-, —C₀-C₃alkyl-C₁-C₆heteroalkyl-aryl-C₀-C₆alkyl-, —C₀-C₃alkyl-C₁-C₆heteroalkyl-heteroaryl-C₀-C₆alkyl-, —C₀-C₆alkyl-cycloalkyl-C₀-C₆alkyl-, —C₀-C₆alkyl-heterocyclyl-C₀-C₆alkyl-, —C₄-C₆heterocyclyl-aryl-C₀-C₆alkyl-, —C₄-C₆heterocyclyl-aryl-C₀-C₆heteroalkyl-, —C₀-C₆alkyl-C₄-C₆heterocyclyl-C₀-C₆alkyl-, —C₀-C₆alkyl-heteroaryl-C₀-C₆alkyl-, —C₀-C₆alkylheteroaryl-C₀-C₆heteroalkyl-, —C₄-C₆heterocyclyl-heteroaryl-C₀-C₆alkyl-, —C₀-C₆alkyl-aryl-C₂-C₆alkynyl-, —C₀-C₆alkyl-heteroaryl-C₂-C₆alkynyl-, —C₀-C₆alkyl-aryl-C₂-C₆alkynyl-C₂-C₆alkenyl-, —C₀-C₆alkyl-aryl-C₂-C₆alkenyl-, —C₀-C₆alkyl-heteroaryl-C₂-C₆alkenyl-, —C₀-C₃alkyl-C₂-C₆alkenyl-aryl-C₀-C₆alkyl-, —C₀-C₃alkyl-C₂-C₆alkenyl-heteroaryl-C₀-C₆alkyl-, —C₀-C₃alkyl-C₂-C₆alkynyl-aryl-C₀-C₆alkyl-, —C₀-C₃alkyl-C₂-C₆alkynyl-heteroaryl-C₀-C₆alkyl-, —C₀-C₆alkylaryl-aryl-C₀-C₆alkyl-, —C₀-C₆alkylaryl-heteroaryl-C₀-C₆alkyl-, —C₀-C₃alkyl-heteroaryl-heteroaryl-C₀-C₃alkyl-, —C₀-C₃alkyl-heteroaryl-aryl-C₀-C₃alkyl-, —C₀-C₃alkyl-aryl-heteroaryl-C₀-C₃alkyl-, —C₀-C₃alkyl-aryl-aryl-C₀-C₃alkyl-, and —C₀-C₆alkyl-C₃-C₆cycloalkyl-C₀-C₆alkyl-, wherein each alkyl, alkenyl, alkynyl, heteroalkyl, aryl, heteroaryl, heterocyclyl, and cycloalkyl moiety is optionally substituted, and wherein when J is ═CH—, Q is a covalent bond and B is attached through a carbon sp² to J;

Q is selected from the group consisting of an optionally substituted:

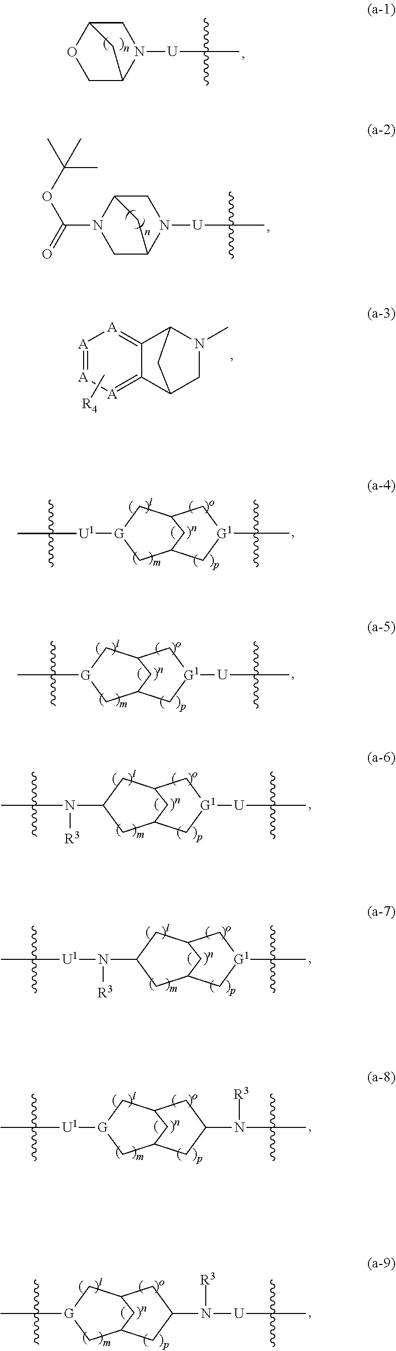

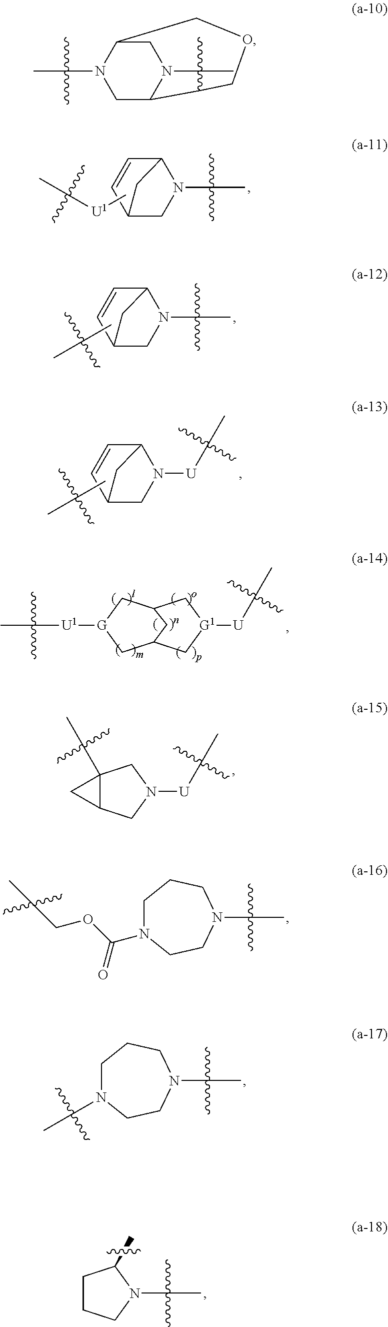

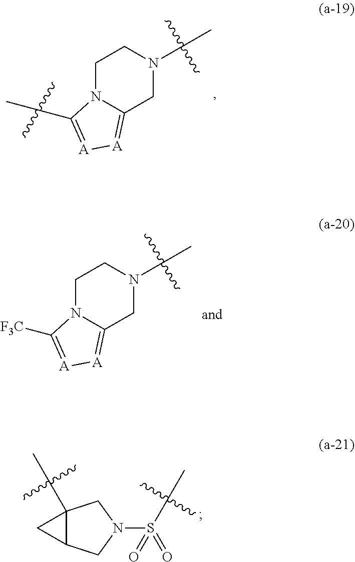

or where possible, an (R,R) or (S,S) enantiomer or a mixture of enantiomers thereof,

wherein G and G¹ are independently selected from carbon and N; the variables I, m, n, o and p denote numbers that are each independently selected from 0, 1, 2 or 3 provided that the sum total of l, m, n, o and p is 4, 5, 6 or 7, such that the group represented by Q comprises a 6, 7, 8 or 9 membered bridged or fused heterocyclyl, respectively, and further provided that when G and G¹ are both N then the sum total of l and o is not zero, and the sum total of m and p is not zero, and wherein n is an integer ranging from 0 to 3; (preferably, Q comprises a 7 or 8-membered ring; in one particular embodiment, n is zero, such that Q comprises a fused bicyclic ring);

U is selected from the group consisting of —C₀-C₈alkyl-C(O)—C₀-C₃alkyl-, —C₁-C₈alkyl-, —C₀-C₈alkyl-N(R³)—C(O)—C₀-C₃alkyl-, —C₀-C₈alkyl-O—C(O)—C₀-C₃alkyl-, —C₀-C₈alkyl-N(R³)—C(S)—C₀-C₃alkyl-, —C₀-C₈alkyl-O—C(S)—C₀-C₃alkyl-, —C₀-C₈alkyl-N(R³)—S(O)₂—C₀-C₃alkyl-, —C₀-C₈alkyl-heterocyclyl-C₀-C₃alkyl-, a covalent bond and —O—C₂-C₄alkyl-; and

U¹ is selected from the group consisting of H, —C(R¹)(R²)—, —C₀-C₈alkyl-C(O)—C₀-C₃alkyl-, —C₁-C₈alkyl-, —C₀-C₈alkyl-N(R³)—C(O)—C₀-C₃alkyl-, —C(R¹)(R²)—N(R³)—C(O)—C₀-C₃alkyl-, —C(R¹)(R²)—C(O)—C₀-C₃alkyl-, —C₀-C₈alkyl-O—C(O)—C₀-C₃alkyl-, —C(R¹)(R²)—O—C(O)—C₀-C₃alkyl-, —C₀-C₈alkyl-N(R³)—C(S)—C₀-C₃alkyl-, —C₀-C₈alkyl-O—C(S)—C₀-C₃alkyl-, —C₀-C₈alkyl-N(R³)—S(O)₂—C₀-C₃alkyl-, —C₀-C₈alkyl-heterocyclyl-C₀-C₃alkyl-, a covalent bond, (R³)(R^{3a})N—C₂-C₄alkyl-, —O—C₂-C₄alkyl-, and R³—O—C₂-C₄alkyl-;

or

Q is selected from the group consisting of a covalent bond, —C₁-C₈alkyl-, —C₁-C₈alkyl-, —C₁-C₈heterocyclyl-, ═N—O—, —C₀-C₆alkyl-N(R³)—C₀-C₃alkyl-, —C₀-C₆alkyl-O—C₀-C₃alkyl-, —C₀-C₆alkyl-S(O)₀₋₂—C₀-C₃alkyl-, —C₀-C₆alkyl-heterocyclyl-C₀-C₃alkyl-, —C₀-C₆alkyl-C(O)—C₀-C₃alkyl-, —C₀-C₆alkyl-O—C₀-C₃alkyl-, —C₀-C₆alkyl-cycloalkyl-C₀-C₃alkyl-, —C₀-C₆alkyl-N(R³)—C(O)-cycloalkyl-C₀-C₃alkyl-, —C₀-C₆alkyl-N(R³)-cycloalkyl-C₀-C₃alkyl-, —C₀-C₆alkyl-S(O)₀₋₂—N(R³)-cycloalkyl-C₀-C₃alkyl-, —C₀-C₆alkyl-N(R³)—C(O)—N(R³)-cycloalkyl-C₀-C₃alkyl-, —C₀-C₆alkyl-O—C(O)—O-cycloalkyl-C₀-C₃alkyl-, —C₀-C₆alkyl-N(R³)—C(O)—O-cycloalkyl-C₀-C₃alkyl-, —C₀-C₆alkyl-(CR³═CR³)₁₋₂—C₀-C₆alkyl-, —C₀-C₆alkyl-(C≡C)₁₋₂—C₀-C₆alkyl-, —C₀-C₆alkyl-N(R³)—C(O)—C₀-C₃alkyl-, —C₀-C₆alkyl-N(R³)—C(O)-alkenyl-C₀-C₄alkyl-, —C₀-C₆alkyl-C(O)—N(R³)—C₀-C₄alkyl-, —C₀-C₆alkyl-SO₂—N(R³)—C₀-C₃alkyl-, —C₀-C₆alkyl-N(R³)—SO₂—C₀-C₃alkyl-, —C₀-C₃alkyl-N(R³)—S(O)₂—N(R³)—C₀-C₃alkyl-, —C₀-C₆alkyl-S—C₀-C₃alkyl-, —C₀-C₆alkyl-S(O)—C₀-C₃alkyl-, —C₀-C₆alkyl-S(O)₂—C₀-C₃alkyl-, —C₀-C₆alkyl-N(R³)—C(O)—N(R³)—C₀-C₃alkyl-, ═N—O—C₀-C₃alkyl-, -heterocyclyl-C₀-C₃alkyl-heterocyclyl-C₀-C₃alkyl-, —SO₂—C₀-C₆alkyl-heterocyclyl-C₀-C₃alkyl-, —C(O)—C₀-C₆alkyl-bridged heterocyclyl-C₀-C₃alkyl-, —N(R³)—C(O)—C₀-C₆alkyl-heterocyclyl-C₀-C₃alkyl-, —O—C(O)—C₀-C₆alkyl-heterocyclyl-C₀-C₃alkyl-, —N(R³)—C(S)—C₀-C₆alkyl-heterocyclyl-C₀-C₃alkyl-, —O—C(S)—C₀-C₆alkyl-heterocyclyl-C₀-C₃alkyl-, —N(R³)—S(O)₂—C₀-C₆alkyl-heterocyclyl-C₀-C₃alkyl-, —C₀-C₆alkyl-heterocyclyl-C₀-C₃alkyl-SO₂—N(R³)—, —C₀-C₆ alkyl-heterocyclyl-C₀-C₃alkyl-C(O)—N(R³)— and —C₀-C₆alkyl-heterocyclyl-C₀-C₃alkyl-C(O)—O—, wherein each alkyl, alkenyl, alkynyl, heteroalkyl, cycloalkyl, heterocyclyl, aryl and heteroaryl moiety is optionally substituted; wherein

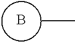

is selected from the group consisting of b-1a to b-1k and b-1 to b-125, and wherein when Q is attached to

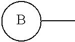

via ═N—O—, or ═N—O—C₀₋₃alkyl, it is attached through carbon in

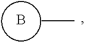

and wherein each alkyl, heteroalkyl, cycloalkyl, heterocyclyl and alkenyl moiety is optionally substituted; and wherein when Q is a covalent bond and J is attached to

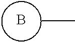

via ═CH—, then it is attached through carbon sp² in

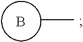

or when

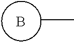

is selected from the group consisting of b-1 to b-121 and is attached to Q via a N in

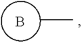

then Q is selected from the group consisting of a covalent bond, —C(O)—C₁-C₃alkyl-O—, —C₁-C₈alkyl-, —C₂-C₆alkyl-N(R³)—C₀-C₃alkyl-, —C₀-C₆alkyl-heterocyclyl-C₀-C₃alkyl-, —C₀-C₆alkyl-C(O)—C₀-C₃alkyl-, —C₀-C₆alkyl-O—C₀-C₃alkyl-, —C₁-C₆alkyl-(CR³═CR³)₁₋₂—C₀-C₆alkyl-, —C₁-C₆alkyl-(C≡C)₁₋₂—C₀-C₆alkyl-, —C₂-C₆alkyl-N(R³)—C(O)—C₀-C₃alkyl, —C₂-C₆alkyl-N(R³)—C(O)-alkenyl-C₀-C₃alkyl, —C₀-C₆alkyl-C(O)—N(R³)—C₀-C₄alkyl-, —C(O)—O—C₀-C₄alkyl, —C₀-C₆alkyl-S(O)₂—N(R³)—C₀-C₃alkyl, —C₂-C₆alkyl-N(R³)—S(O)₂—C₀-C₃alkyl, —C₂-C₃alkyl-N(R³)—S(O)₂—N(R³)—C₀-C₃alkyl-, —C₂-C₆alkyl-S—C₀-C₃alkyl, —C₂-C₆alkyl-S(O)—C₀-C₃alkyl, —C₀-C₆alkyl-S(O)₂—C₀-C₃alkyl, —C₂-C₆alkyl-N(R³)—C(O)—N(R³)—C₀-C₃alkyl, —C₂-C₃alkyl-C═N—O—C₀-C₃alkyl, —SO₂—C₀-C₆alkyl-heterocyclyl-C₀-C₃alkyl-, —C(O)—C₀-C₆alkyl-heterocyclyl-C₀-C₃alkyl-, —C₂-C₄alkyl-N(R³)—C(O)—C₀-C₆alkyl-heterocyclyl-C₀-C₃alkyl-, —C₂-C₄alkyl-O—C(O)—C₀-C₆alkyl-heterocyclyl-C₀-C₃alkyl-, —C₂-C₄alkyl-N(R³)—C(S)—C₀-C₆alkyl-heterocyclyl-C₀-C₃alkyl-, —C₂-C₄alkyl-O—C(S)—C₀-C₆alkyl-heterocyclyl-C₀-C₃alkyl-, —C₂-C₄alkyl-N(R³)—S(O)₂—C₀-C₆alkyl-heterocyclyl-C₀-C₃alkyl-, —C₀-C₆alkyl-heterocyclyl-C₀-C₃alkyl-S(O₂)—N(R³)—, —C₀-C₆alkyl-heterocyclyl-C₀-C₃alkyl-C(O)—N(R³)— and —C₀-C₆alkyl-heterocyclyl-C₀-C₃alkyl-C(O)—O—, wherein each alkyl, heterocyclyl and alkenyl moiety is optionally substituted, and wherein the heterocyclyl moiety is optionally bridged with —(CH₂)₀₋₃—;

R¹ and R² are independently selected from the group consisting of —H, C₁-C₆alkyl, aryl, heteroaryl, heterocyclyl, cycloalkyl and a protecting group;

each R³ is independently selected from the group consisting of —H, alkyl, C₀-C₃alkyl-heterocyclyl, C₁-C₃alkyl-C₂-C₆alkenyl, C₁-C₃alkyl-C₂-C₃alkynyl, —C₂-C₄alkyl-OR¹, —C₂-C₄alkyl-NR^{3b}R^{3c}, —C₂-C₄alkyl-NR¹R², heteroalkyl, C₀-C₆alkylheteroaryl, C(O)CF₃, —C(O)—NH₂, —C(O)—NR^{3b}R^{3c}, —C(O)—NR¹R², —C(O)—OR¹, —S(O)₂—NR¹R², —S(O)₂—R¹, —C(O)—R¹, —C₃-C₆cycloalkyl, —C₀-C₃alkyl-C₃-C₇cycloalkyl, —C₁-C₆alkylaryl, aryl, C₀-C₃alkyl-heteroaryl and heteroaryl, wherein each alkyl, alkenyl, alkynyl, heteroalkyl, cycloalkyl, heterocyclyl, aryl and heteroaryl moiety is optionally substituted with from one to three independently selected substituents;

each R^{3a} is independently selected from the group consisting of —H, alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl, covalent bond, wherein each alkyl, alkenyl, alkynyl, heteroalkyl, cycloalkyl, heterocyclyl, aryl and heteroaryl moiety is optionally substituted;

wherein R³ and R^{3a}, together with the atom to which they are attached, optionally form a heterocyclic ring, wherein the heterocyclyl moiety is optionally substituted;

wherein R^{3b} and R^{3c}, together with the atom to which they are attached, optionally form a heterocyclic ring, wherein the heterocyclyl moiety is optionally substituted; provided that

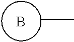

is absent when Q is structure (a-1), (a-2), (a-3), (a-20) or when U¹ is H, N(R³)(R^{3a})—C₂-C₄alkyl- or R³—O—C₂-C₄alkyl-;

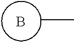

is selected from the group consisting of hydrogen, aryl, aryl-alkyl-, heteroaryl, heteroaryl-alkyl-, heterocyclyl, cycloalkyl, heterocyclyl-alkyl, cycloalkyl-alkyl, C₁-C₁₀alkyl, (aryl)₂-CH—C₀-C₆alkyl-, (aryl)(heteroaryl)CH—C₀-C₆alkyl- and (heteroaryl)₂CH—C₀-C₆alkyl-, each of which is optionally substituted; or

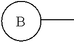

is a radical selected from the group consisting of

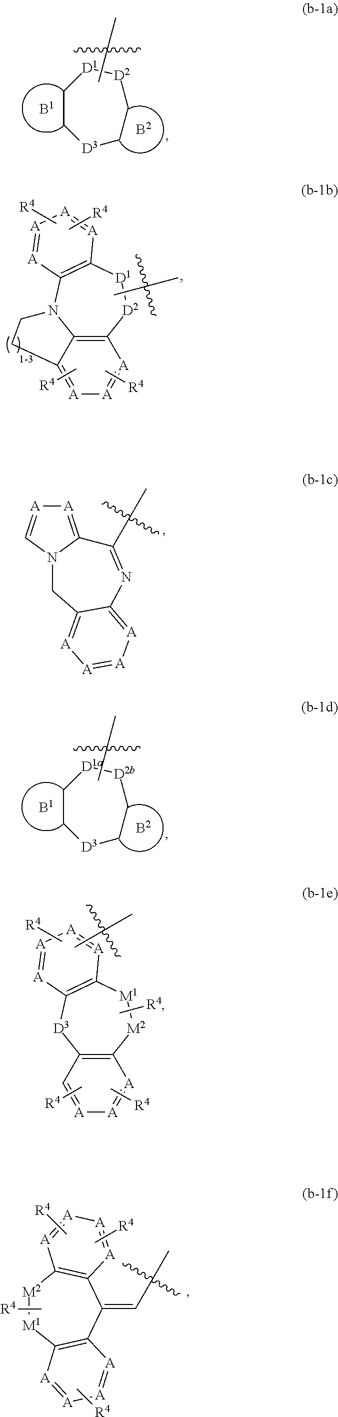

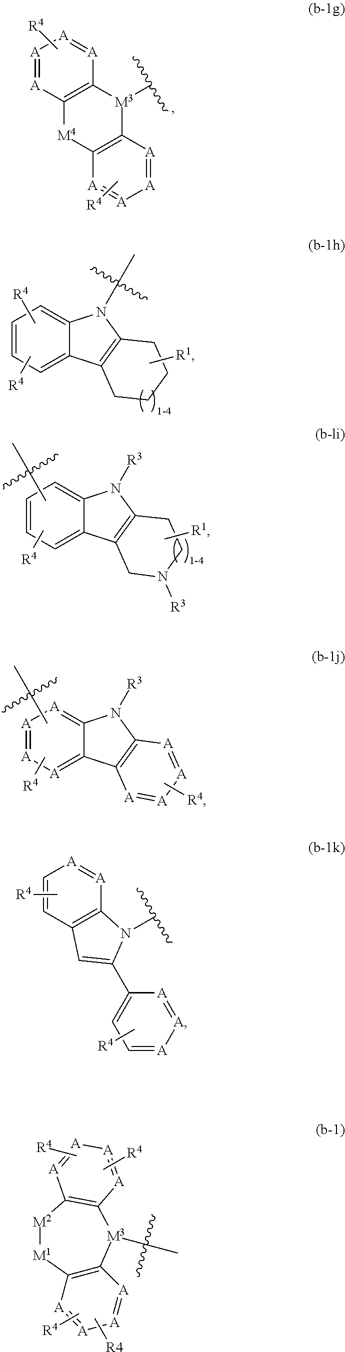

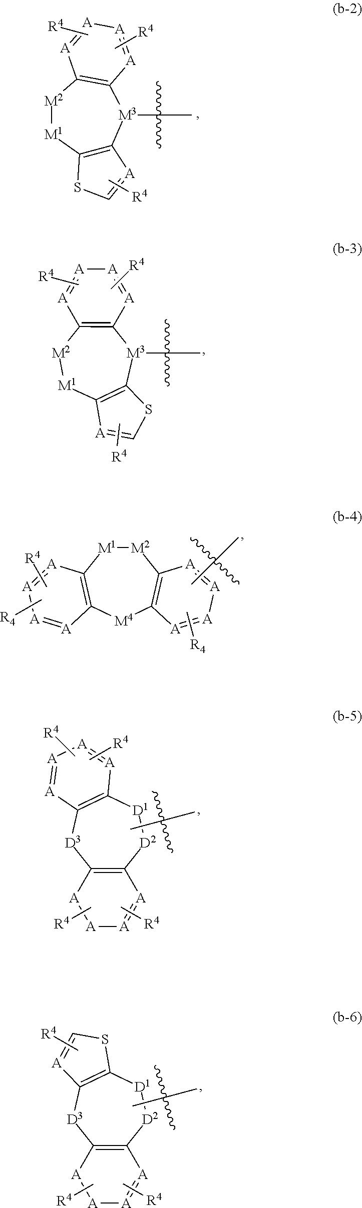

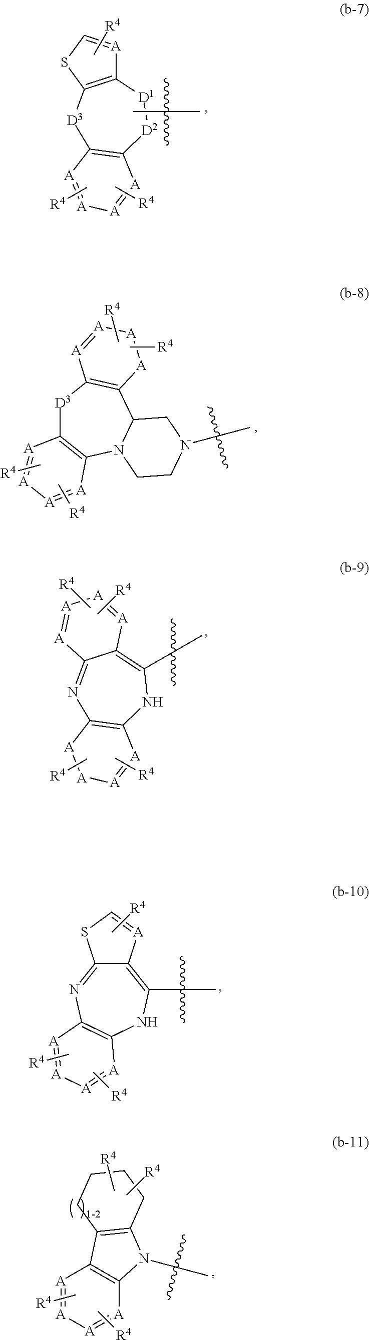

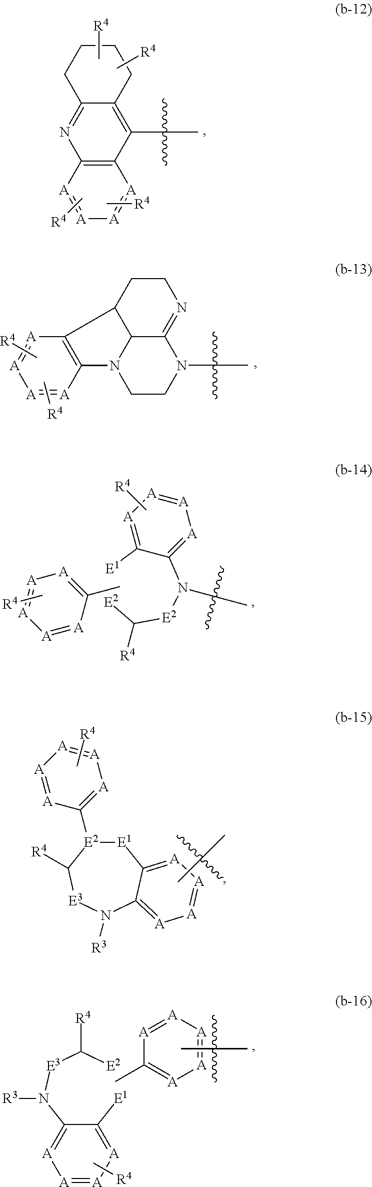

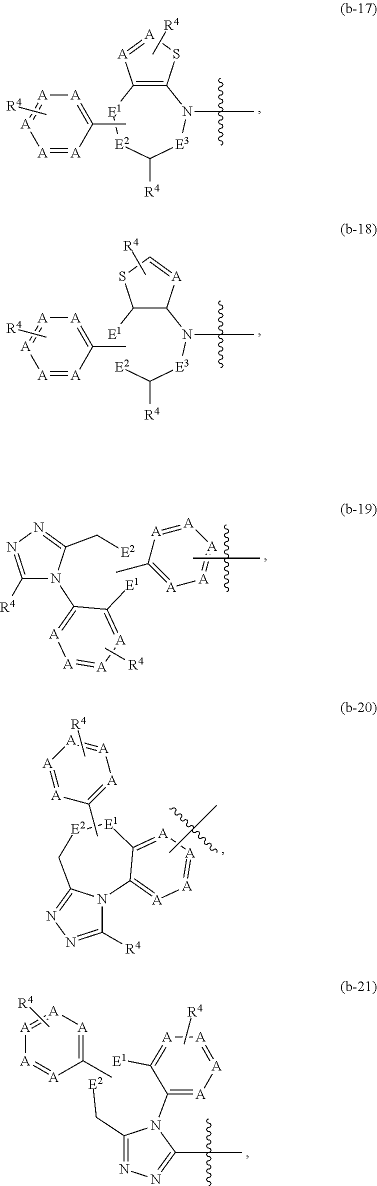

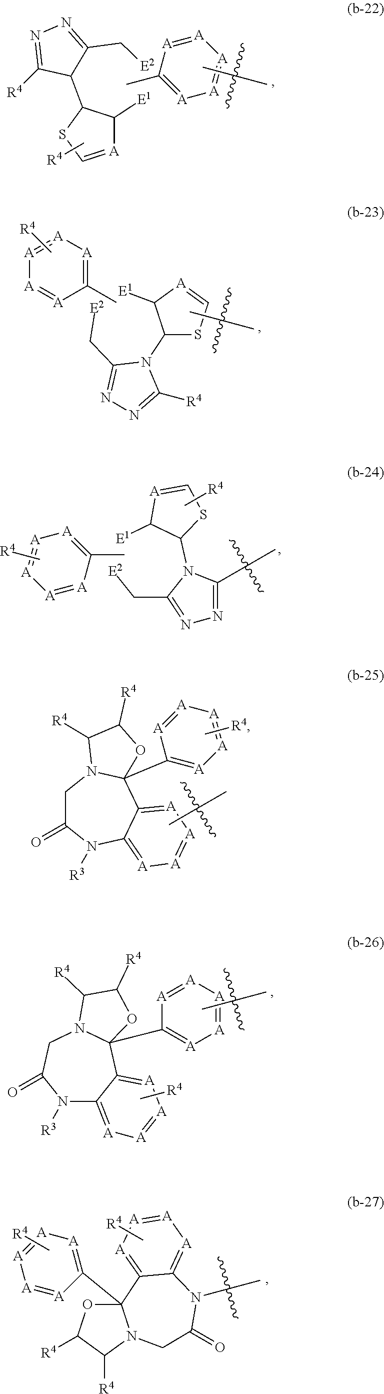

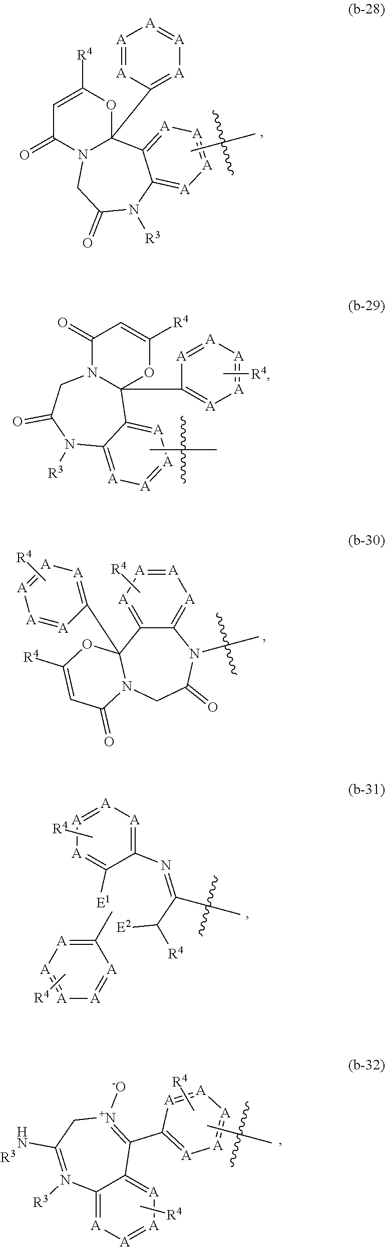

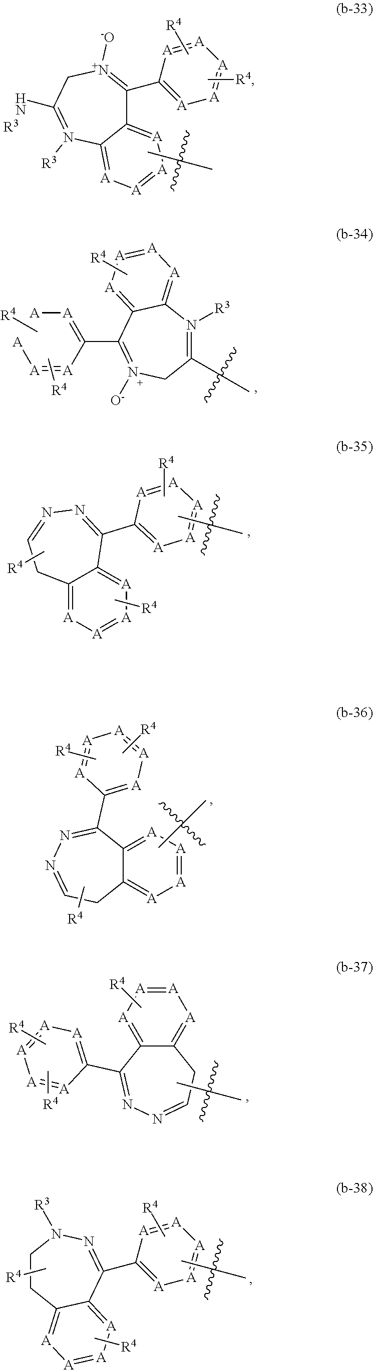

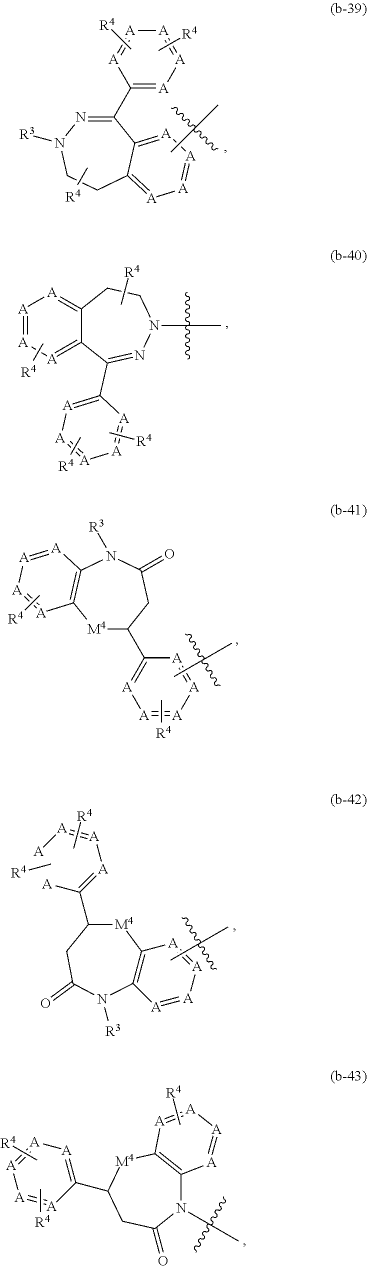

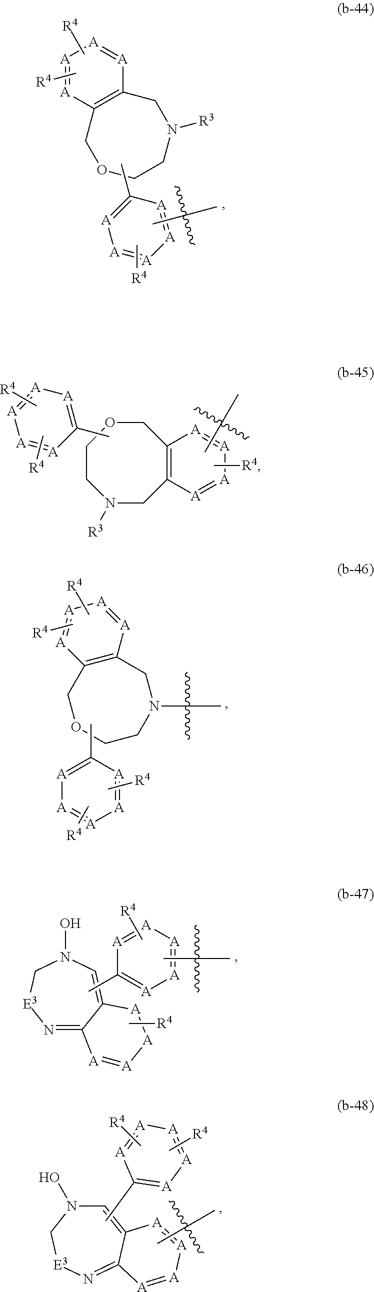

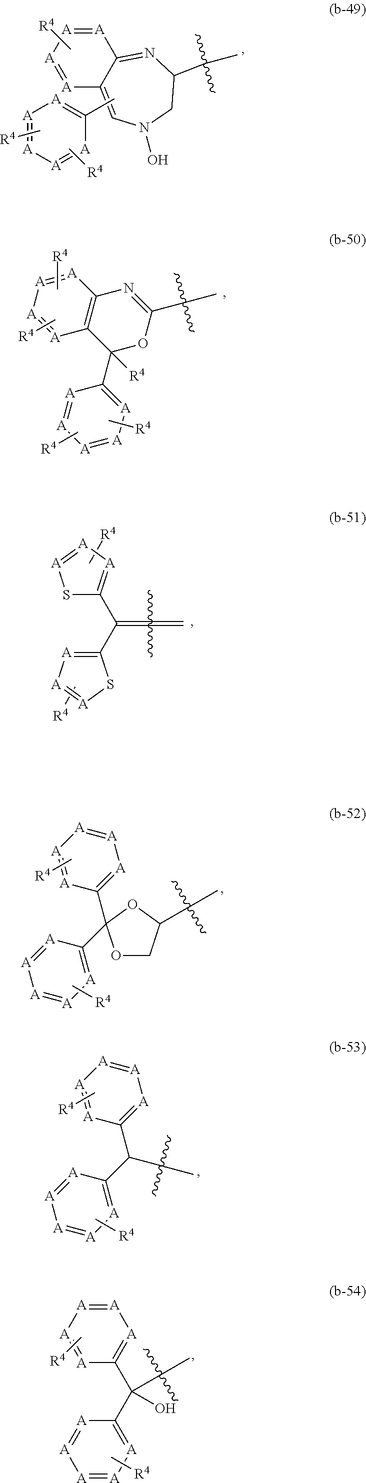

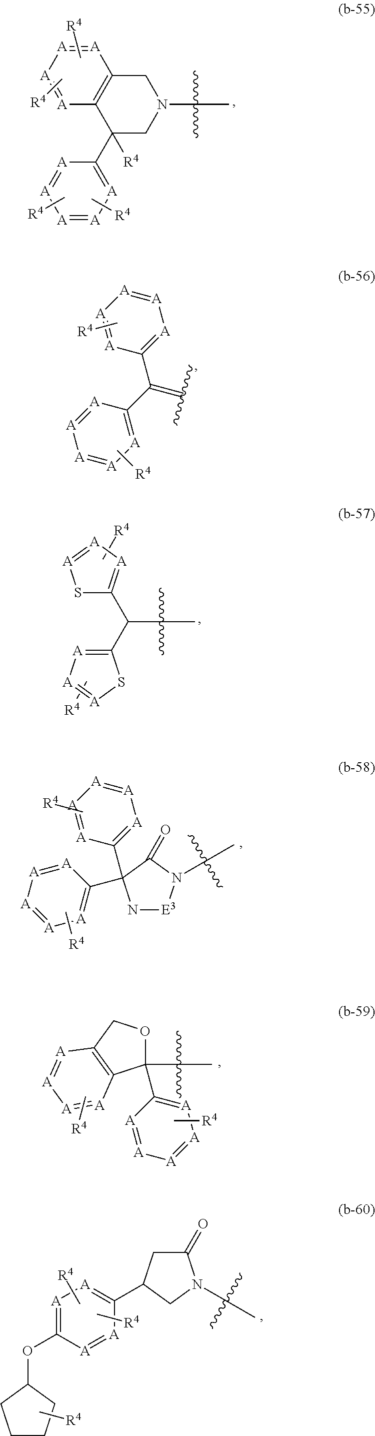

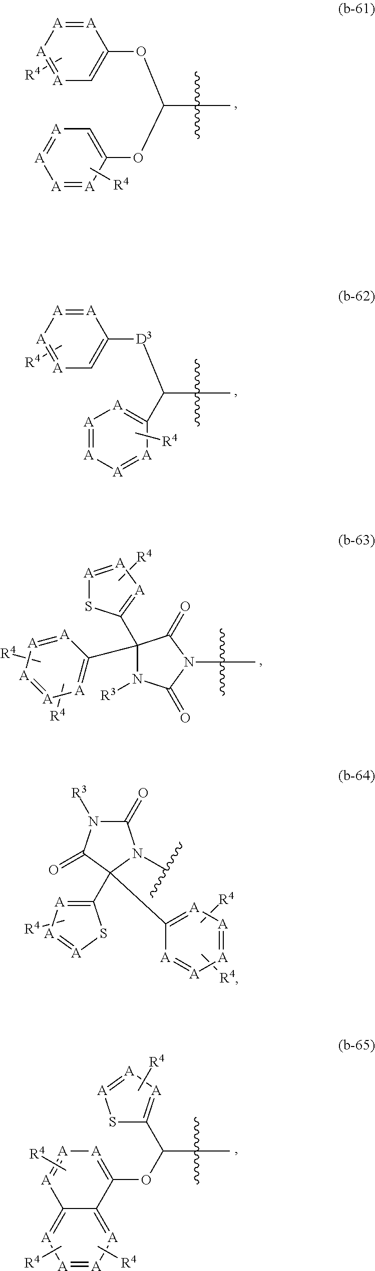

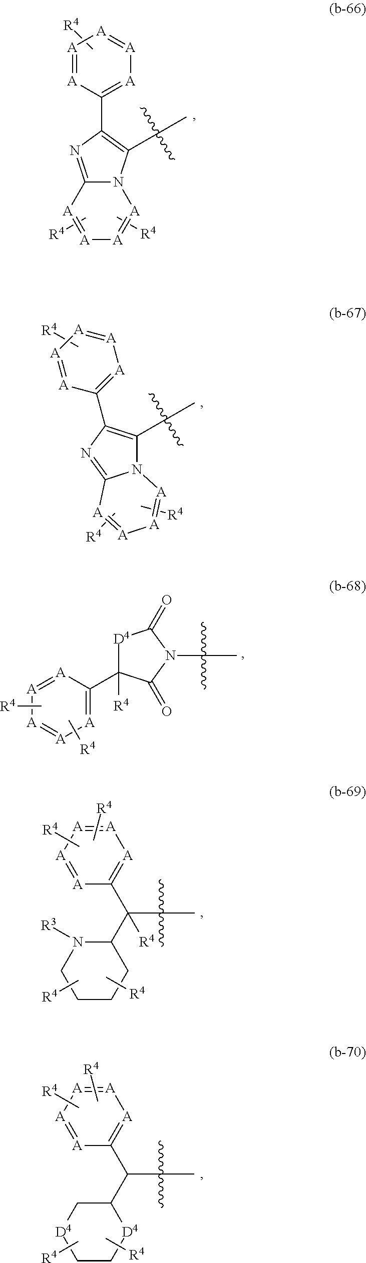

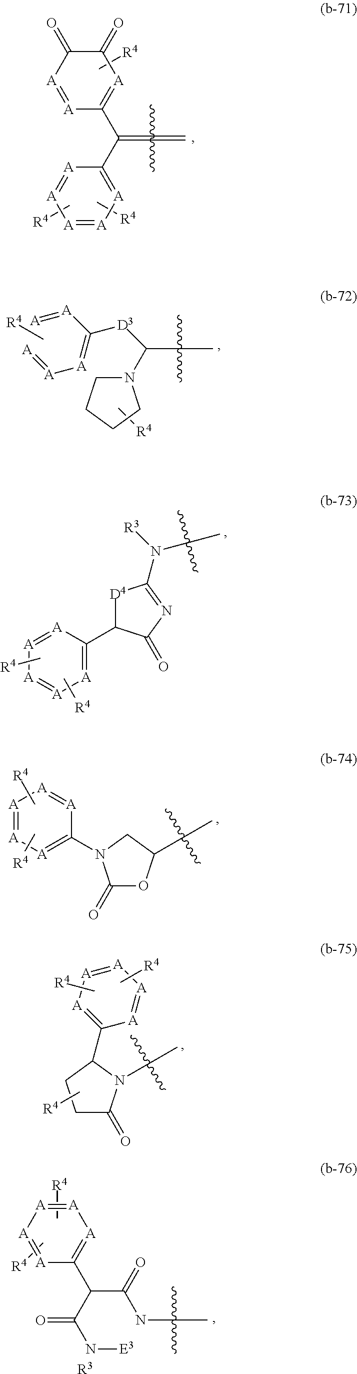

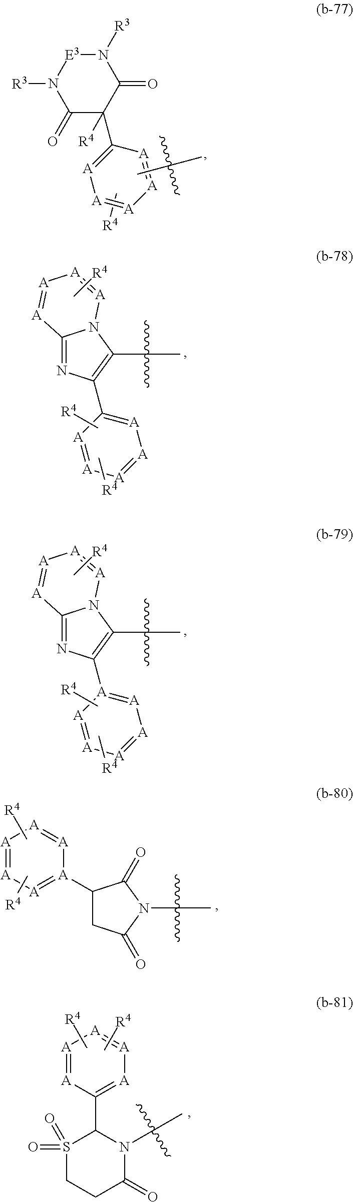

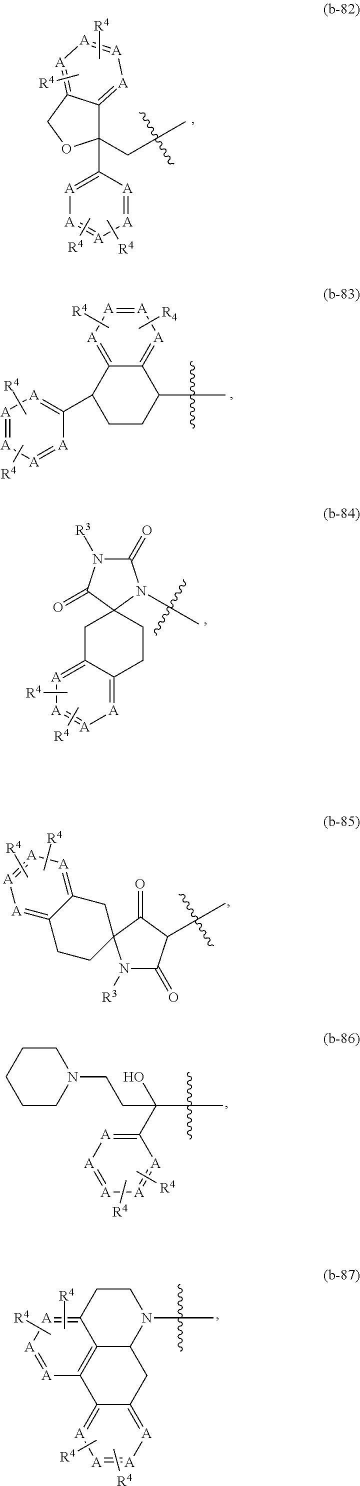

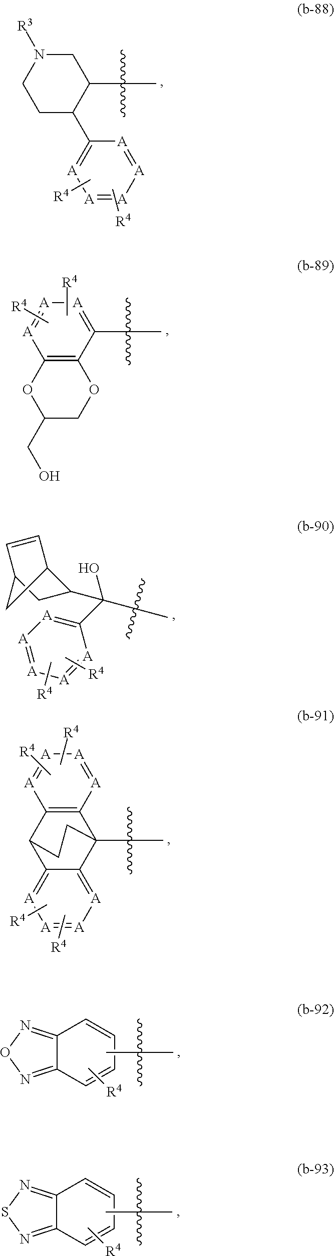

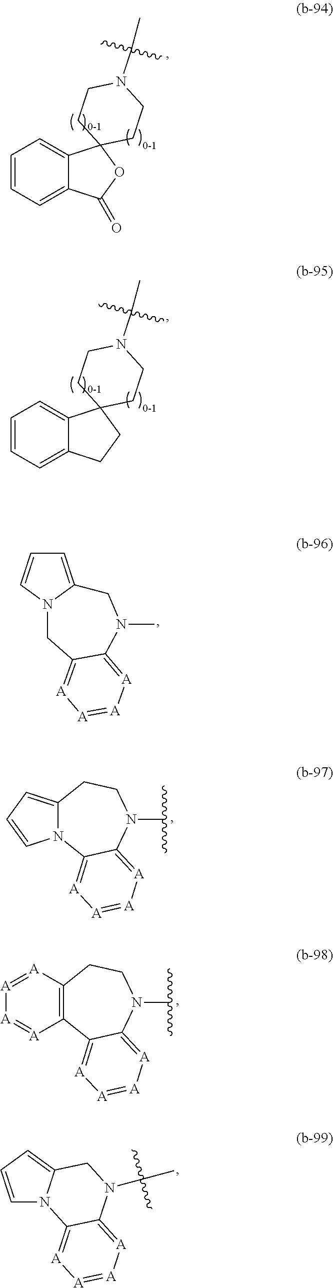

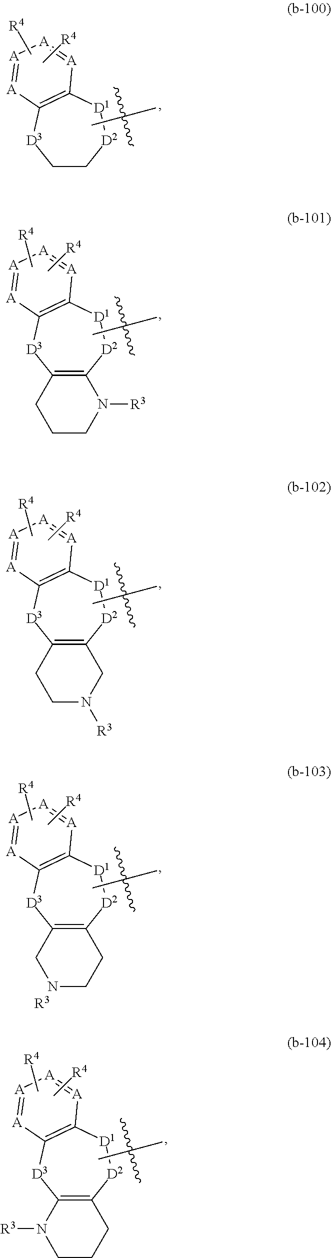

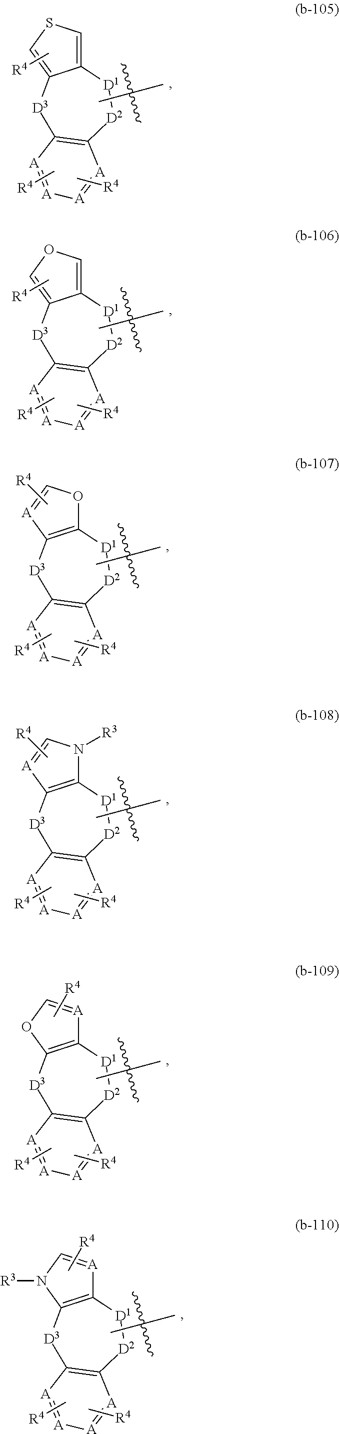

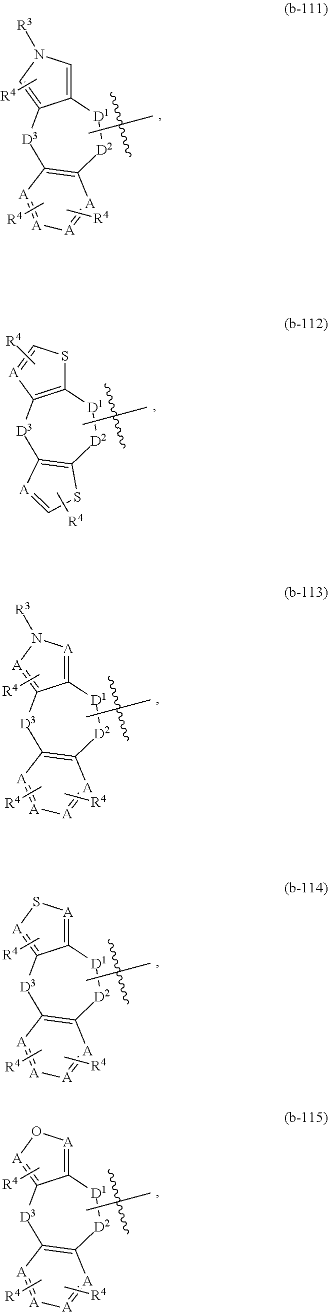

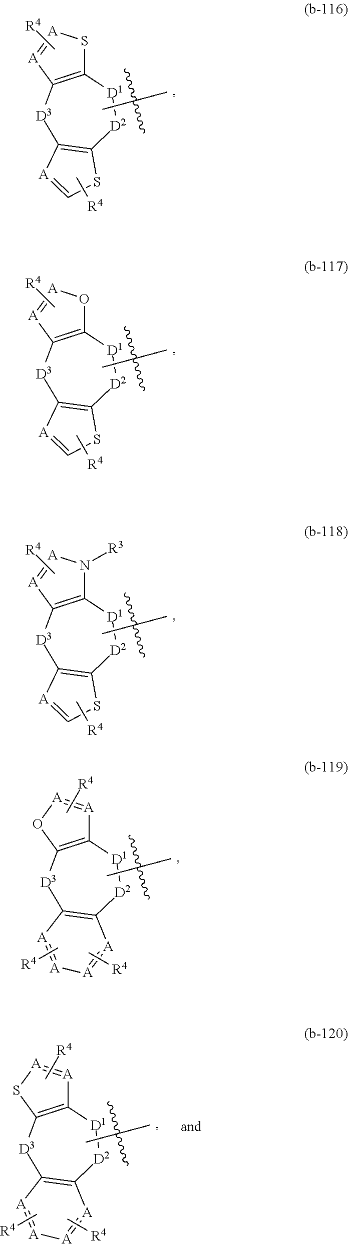

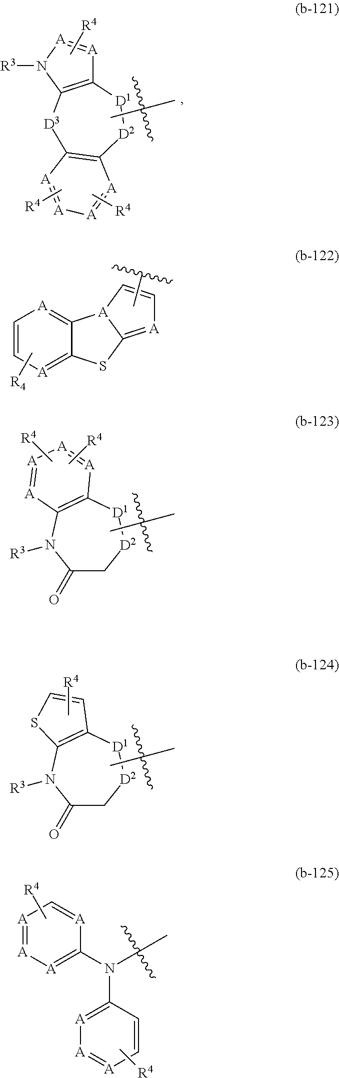

wherein

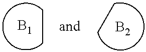

and are independently selected from phenyl, a 5- or 6-membered heteroaryl and heterocyclyl, each of which is optionally substituted with one to three independently selected substituents;

provided that when

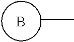

is selected from the group consisting of hydrogen, aryl, aryl-alkyl-, heteroaryl, heteroaryl-alkyl-, heterocyclyl, cycloalkyl, heterocyclyl-alkyl, cycloalkyl-alkyl, C₁-C₁₀alkyl, (aryl)₂-CH—C₀-C₆alkyl-, (aryl)(heteroaryl)CH—C₀-C₆alkyl- and (heteroaryl)₂CH—C₀-C₆alkyl-, each of which is optionally substituted, then Q is selected from the group consisting of a-3, a-4, a-5, a-6, a-7, a-8, a-9, a-10, a-11, a-12, a-13 and a-14,

wherein

each A is independently selected from the group consisting of N, —N-oxide, —CH═ and —C(R⁴)═, wherein no more than two A per 5 or 6 membered ring are N in a

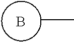

group, and wherein no more than one A is —N-oxide;

the group M¹-M² is selected from the group consisting of a covalent bond, —N(R³)CH₂—, —CH₂N(R³)—, —S(O)₀₋₂—CH₂—, —CH₂S(O)₀₋₂—, —O—CH₂—, —CH₂—O—, —C(O)N(R³)—, —C(O)—O—, —C(O)—CH₂—, —CH(OH)—CH₂—, —CH(F)—CH₂—, —CH₂—C(O)—, —CH₂—CH(OH)—, —CH₂—CH(F)—, —N(R³)—C(O)—, —SO₂N(R³)—, —N(R³)SO₂—, —CH(R⁴)CH₂—, —CH₂CH(R⁴)—, —N═C(R⁴)—, —C(R⁴)═N—, —CH₂—CH₂—, —CH═CH—, —CH(R³)—CH(R³)—, —C(R³)═C(R³)—, —C(R⁴)═C(R⁴)—, —CF═CH—, —CH═CF—,

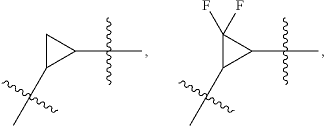 [SMILES: *C1CC1* ; *C1C(*)C1(F)F]

—CH₂—, —C(R³)(R^{3a})—, —S(O)₀₋₂—, —N(R³)—, or absent;

M³ is selected from the group consisting of

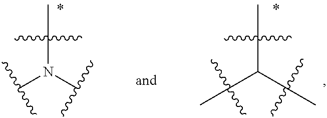

or M³ is

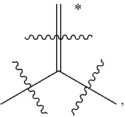

wherein Q is attached to

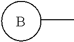

via ═N—O—, or ═N—O—C₀₋₃alkyl, or J is attached to

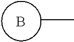

### via ═CH—,

wherein * represents the point of attachment to Q;

M⁴ is selected from the group consisting of

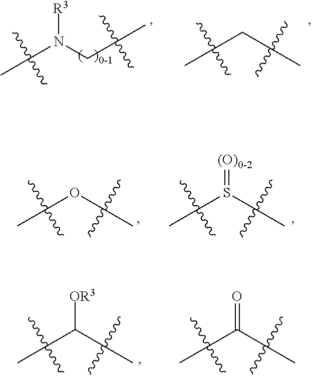

and covalent bond;

wherein, when M¹-M² is a covalent bond, M⁴ is selected from the group consisting of

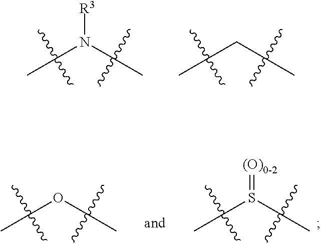

the groups D¹-D² and D^{1a}-D^{2a} are selected from the group consisting of

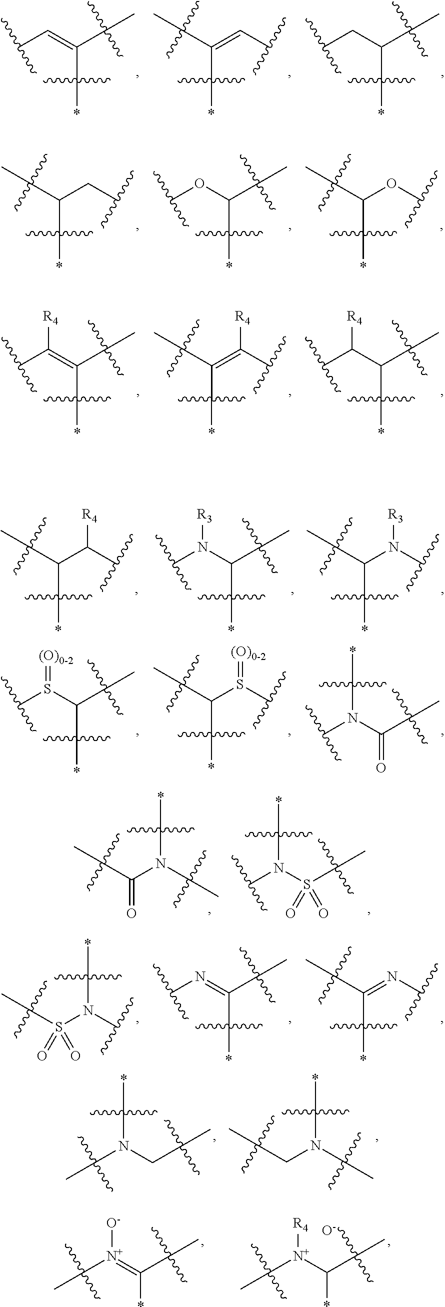

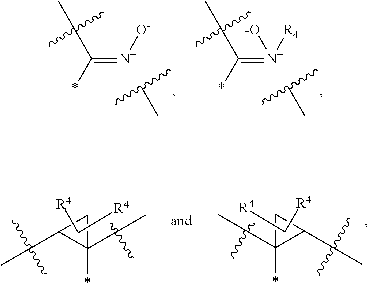

wherein, * represents the point of attachment to Q;

D³ is selected from the group consisting of a covalent bond,

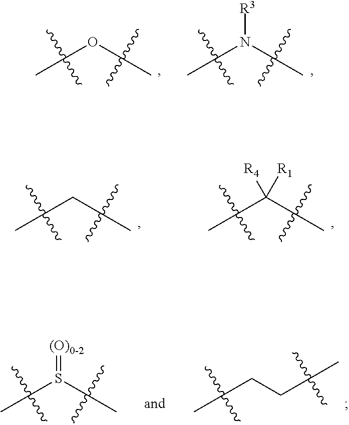

wherein the

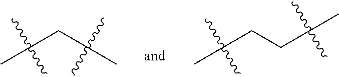

are optionally substituted;

D⁴ is selected from the group consisting of

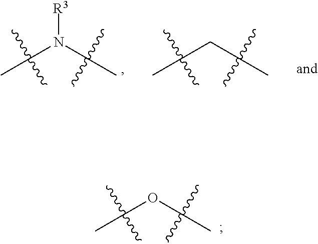

wherein the

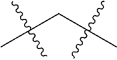 [SMILES: *C*]

is optionally substituted;

the group E¹-E² is selected from the group consisting of

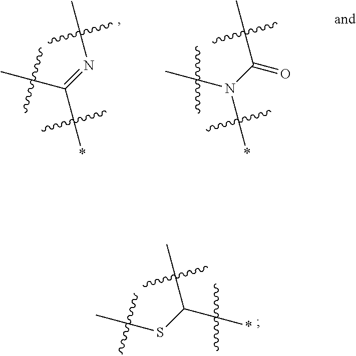

wherein * represents the point of attachment to Q; and

E³ is selected from the group consisting of —C(O)—, —C(S)—, —CH₂—, —C(OH)₂— and —C═N(R³)—;

and

R⁴ is independently selected from the group consisting of —H, C₁-C₆alkyl, C₂-C₆alkenyl, C₂-C₆alkynyl, C₁-C₆alkyl-R³, —C₀-C₆alkyl-OR³, —C₀-C₆alkyl-OR¹, —C₀-C₆alkyl-C(O)—OR³, —C₀-C₆alkyl-C(O)NR³R^{3a}, —CH═CH—C(O)—OR³, —CH═CH—C(O)—N(R³)(R^{3a}), —N(R³)—C(O)—CF³, —N(R³)—C₂-C₆alkyl-N(R³)(R^{3a}), —C₀-C₆alkyl-N(R³)(R^{3a}), —N(R³)—C(O)—C₁-C₆alkyl-R³, —N(R³)—S(O)₂—C₁-C₆alkyl-R³, —S(O)₂—N(R³)(R^{3a}), —O—C₂-C₆alkyl-N(R³)(R^{3a}), —O—C₂-C₆alkyl-OR¹, —S—R³, —S(O)—C₁-C₆alkyl-R³, —S(O)₂—C₁-C₆alkyl-R³, C₃-C₆cycloalkyl, heterocyclyl, C₄-C₇heterocyclyl-R³, —O—C₂-C₄alkyl-heterocyclyl, —O-heterocyclyl-C(O)—OR³, —O—C₀-C₄alkyl-aryl, —O—C₀-C₄alkyl-heteroaryl, —O—C(O)—NR³—C₀-C₄alkyl-aryl, —O—C(O)—NR³—C₀-C₄alkyl-heteroaryl, —O—C₀-C₄alkyl-heterocyclylaryl, —O—C₀-C₄alkyl-heterocyclyl-heteroaryl, —N(R³)—C₂-C₄alkyl-heterocyclyl, —N(R³)C(O)N(R³)—C₀-C₄alkyl-heterocyclyl-R³, —C₀-C₄alkyl-OC(O)—R³, —C₀-C₄alkyl-N(R³)C(O)—O—R³, —C₀-C₄alkyl-heterocyclyl-C(O)—O—R³, —N(R³)—C₂-C₄alkyl-heterocyclyl, F, Cl, Br, I, NO₂, —CF₃, —OCF₃, —OCHF₂, —SCF₃, —SF₅, —SO₃H, —CN, —C₁-C₆ alkylaryl, aryl, heteroaryl, cycloalkyl, —C₁-C₆ alkylheteroaryl, wherein each alkyl, alkenyl, alkynyl, cycloalkyl, heterocyclyl, aryl and heteroaryl moeity of the aforementioned R⁴ is optionally substituted;

or

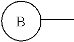

is selected from the group consisting of structures b-1a to b-1k and (b-1) to (b-125) and Q-J-L taken together is selected from the group consisting of —C₃-C₈alkyl-, —C(O)—C₃-C₈alkyl-, —C₀-C₃alkyl-O—C₃-C₈alkyl-, —C₀-C₃alkyl-C₁-C₄alkenyl-C₀-C₃alkyl-, ═N—O—C₁-C₈alkyl-, ═N—O—C₀-C₃alkyl-aryl-C₀-C₃alkyl-, ═N—O—C₀-C₃alkyl-aryl-C₀-C₃alkenyl-, ═N—O—C₀-C₃alkyl-aryl-C₀-C₃alkynyl-, ═N—O—C₀-C₃alkyl-heteroaryl-C₀-C₃alkyl-, ═N—O—C₀-C₃alkyl-heteroaryl-C₀-C₃alkenyl-, ═N—O—C₀-C₃alkyl-heteroaryl-C₀-C₃alkynyl-, —C₀-C₃alkyl-aryl-C₀-C₃alkyl-, —C₀-C₃alkyl-aryl-C₂-C₄alkenyl-, —C₀-C₃alkyl-aryl-C₂-C₄alkynyl-, —C₀-C₃alkyl-heteroaryl-C₀-C₃alkyl-, —C₀-C₃alkyl-heteroaryl-C₁-C₃alkenyl-, —C₀-C₃alkyl-heteroaryl-C₁-C₃alkynyl-, —C₀-C₃alkyl-N(R³)— C₀-C₃alkyl-aryl-C₀-C₃alkyl-, —C₀-C₃alkyl-N(R³)—C₀-C₃alkyl-aryl-C₂-C₃alkenyl-, —C₀-C₃alkyl-N(R³)—C₀-C₃alkyl-aryl-C₂-C₃alkynyl-, —C₀-C₃alkyl-N(R³)—C₀-C₃alkyl-heteroaryl-C₀-C₃alkyl-, —C₀-C₃alkyl-N(R³)—C₀-C₃alkyl-heteroaryl-C₂-C₃alkenyl-, —C₀-C₃alkyl-N(R³)—C₀-C₃alkyl-heteroaryl-C₂-C₃alkynyl-, —C₀-C₃alkyl-C(O)—N(R³)—C₀-C₃alkyl-aryl-C₀-C₃alkyl-, —C₀-C₃alkyl-N(R³)—C(O)—C₀-C₃alkyl-aryl-C₀-C₃alkyl-, —C₀-C₃alkyl-C(O)—N(R³)—C₀-C₃alkyl-aryl-C₂-C₃alkenyl-, —C₀-C₃alkyl-N(R³)—C(O)—C₀-C₃alkyl-aryl-C₂-C₃alkenyl-, —C₀-C₃alkyl-C(O)—N(R³)—C₀-C₃alkyl-aryl-C₂-C₃alkynyl-, —C₀-C₃alkyl-N(R³)—C(O)—C₀-C₃alkyl-aryl-C₂-C₃alkynyl-, —C₀-C₃alkyl-C(O)—N(R³)—C₀-C₃alkyl-heteroaryl-C₀-C₃alkyl-, —C₀-C₃alkyl-N(R³)—C(O)—C₀-C₃alkyl-heteroaryl-C₀-C₃alkyl-, —C₀-C₃alkyl-C(O)—N(R³)—C₀-C₃alkyl-heteroaryl-C₂-C₃alkenyl-, —C₀-C₃alkyl-N(R³)—C(O)—C₀-C₃alkyl-heteroaryl-C₂-C₃alkenyl-, —C₀-C₃alkyl-C(O)—N(R³)—C₀-C₃alkyl-heteroaryl-C₂-C₃alkynyl-, —C₀-C₃alkyl-N(R³)—C(O)—C₀-C₃alkyl-heteroaryl-C₂-C₃alkynyl-, —C₀-C₃alkyl-heterocyclyl-C₀-C₃alkyl-aryl-C₀-C₃alkyl-, —C₀-C₃alkyl-C(O)-heterocyclyl-C₀-C₃alkyl-aryl-C₀-C₃alkyl-, —C₀-C₃alkyl-N(R³)—C(O)-heterocyclyl-C₀-C₃alkyl-aryl-C₀-C₃alkyl-, —C₀-C₃alkyl-O—C(O)-heterocyclyl-C₀-C₃alkyl-aryl-C₀-C₃alkyl-, —C₀-C₃alkyl-heterocyclyl-C₀-C₃alkyl-aryl-C₂-C₄alkenyl, —C₀-C₃alkyl-C(O)-heterocyclyl-C₀-C₃alkyl-aryl-C₂-C₄alkenyl, —C₀-C₃alkyl-N(R³)—C(O)-heterocyclyl-C₀-C₃alkyl-aryl-C₂-C₄alkenyl, —C₀-C₃alkyl-O—C(O)-heterocyclyl-C₀-C₃alkyl-aryl-C₂-C₄alkenyl, —C₀-C₃alkyl-heterocyclyl-C₀-C₃alkyl-aryl-C₂-C₄alkynyl, —C₀-C₃alkyl-C(O)-heterocyclyl-C₀-C₃alkyl-aryl-C₂-C₄alkynyl, —C₀-C₃alkyl-N(R³)—C(O)-heterocyclyl-C₀-C₃alkyl-aryl-C₂-C₄alkynyl, —C₀-C₃alkyl-O—C(O)-heterocyclyl-C₀-C₃alkyl-aryl-C₂-C₄alkynyl, —C₀-C₃alkyl-heterocyclyl-C₀-C₃alkyl-heteroaryl-C₀-C₃alkyl, —C₀-C₃alkyl-C(O)-heterocyclyl-C₀-C₃alkyl-heteroaryl-C₀-C₃alkyl, —C₀-C₃alkyl-N(R³)—C(O)-heterocyclyl-C₀-C₃alkyl-heteroaryl-C₀-C₃alkyl, —C₀-C₃alkyl-O—C(O)-heterocyclyl-C₀-C₃alkyl-heteroaryl-C₀-C₃alkyl, —C₀-C₃alkyl-heterocyclyl-C₁-C₃alkyl-heteroaryl-C₂-C₃alkenyl-, —C₀-C₃alkyl-C(O)-heterocyclyl-C₁-C₃alkyl-heteroaryl-C₂-C₃alkenyl-, —C₀-C₃alkyl-N(R³)—C(O)-heterocyclyl-C₁-C₃alkyl-heteroaryl-C₂-C₃alkenyl-, —C₀-C₃alkyl-O—C(O)-heterocyclyl-C₁-C₃alkyl-heteroaryl-C₂-C₃alkenyl-, —C₀-C₃alkyl-heterocyclyl-C₁-C₃alkyl-heteroaryl-C₂-C₃alkynyl-, —C₀-C₃alkyl-C(O)-heterocyclyl-C₁-C₃alkyl-heteroaryl-C₂-C₃alkynyl-, —C₀-C₃alkyl-N(R³)—C(O)-heterocyclyl-C₁-C₃alkyl-heteroaryl-C₂-C₃alkynyl-, —C₀-C₃alkyl-O—C(O)-heterocyclyl-C₁-C₃alkyl-heteroaryl-C₂-C₃alkynyl-, —C₂-C₄alkyl-O—C₀-C₃alkyl-aryl-, —C₂-C₄alkyl-O—C₀-C₃alkyl-aryl-C₀-C₃alkyl-, —C₂-C₄alkyl-O—C₀-C₃alkyl-aryl-C₂-C₄alkenyl, —C₂-C₄alkyl-O—C₀-C₃alkyl-aryl-C₂-C₄alkynyl, —C₂-C₄alkyl-O—C₀-C₃alkyl-heteroaryl-C₀-C₃alkyl, —C₂-C₄alkyl-O—C₁-C₃alkyl-heteroaryl-C₂-C₃alkenyl-, —C₂-C₄alkyl-O—C₁-C₃alkyl-heteroaryl-C₂-C₃alkynyl-, —C₀-C₆alkyl-U-bridged heterocyclyl-heteroaryl-C₀-C₆alkyl-, —C₀-C₆alkyl-U-bridged heterocyclyl-N(R³)-heteroaryl-C₀-C₆alkyl-, —C₀-C₆alkyl-U—N(R³)-bridged heterocyclyl-heteroaryl-C₀-C₆alkyl-, —C₀-C₆alkyl-U-bridged heterocyclyl-aryl-C₀-C₆alkyl-, —C₀-C₆alkyl-U-bridged heterocyclyl-N(R³)-aryl-C₀-C₆alkyl-, —C₀-C₆alkyl-U—N(R³)-bridged heterocyclyl-aryl-C₀-C₆alkyl-, —C₀-C₆alkyl-U-bridged heterocyclyl-aryl-C₂-C₆alkenyl-, —C₀-C₆alkyl-U-bridged heterocyclyl-N(R³)-aryl-C₂-C₆alkenyl-, —C₀-C₆alkyl-U—N(R³)-bridged heterocyclyl-aryl-C₂-C₆alkenyl-, —C₀-C₆alkyl-U-bridged heterocyclyl-heteroaryl-C₂-C₆alkenyl-, —C₀-C₆alkyl-U-bridged heterocyclyl-N(R³)-heteroaryl-C₂-C₆alkenyl-, —C₀-C₆alkyl-U—N(R³)— bridged heterocyclyl-heteroaryl-C₂-C₆alkenyl-, —C₀-C₆alkyl-bridged heterocyclyl-U-heteroaryl-C₀-C₆alkyl-, —C₀-C₆alkyl-N(R³)-bridged heterocyclyl-U-heteroaryl-C₀-C₆alkyl-, —C₀-C₆alkyl-bridged heterocyclyl-N(R³)—U-heteroaryl-C₀-C₆alkyl-, —C₀-C₆alkyl-bridged heterocyclyl-U-aryl-C₀-C₆alkyl-, —C₀-C₆alkyl-N(R³)-bridged heterocyclyl-U-aryl-C₀-C₆alkyl-, —C₀-C₆alkyl-bridged heterocyclyl-N(R³)—U-aryl-C₀-C₆alkyl-, —C₀-C₆alkyl-bridged heterocyclyl-U-aryl-C₂-C₆alkenyl-, —C₀-C₆alkyl-N(R³)-bridged heterocyclyl-U-aryl-C₂-C₆alkenyl-, —C₀-C₆alkyl-bridged heterocyclyl-N(R³)—U-aryl-C₂-C₆alkenyl-, —C₀-C₆alkyl-bridged heterocyclyl-U-heteroaryl-C₂-C₆alkenyl-, —C₀-C₆alkyl-N(R³)-bridged heterocyclyl-U-heteroaryl-C₂-C₆alkenyl-, and —C₀-C₆alkyl-bridged heterocyclyl-N(R³)—U-heteroaryl-C₂-C₆alkenyl-, wherein each alkyl, alkenyl, aryl, alkynyl, heteroaryl and heterocyclyl moiety is optionally substituted; and wherein the bridge is methylene or propylene;

provided that Formula (I) excludes those compounds wherein

-Q-J-L-C(O)Z is optionally substituted —C₁-C₁₃alkyl-N(R³)—C₀-C₆alkyl-aryl-C₂alkenyl-C(O)NHOH; and

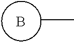

is selected from the group consisting of aromatic polycycles, non-aromatic polycycles, mixed aryl and non-arylpolycycles, polyheteroaryl, non-aromatic polyheterocycles, and mixed aryl and non-aryl polyheterocycles, each of which is optionally substituted;

and

provided that Formula (I) excludes compounds of Formula (A)

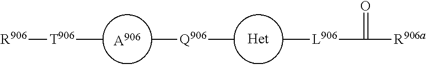

wherein R⁹⁰⁶ is selected from the group consisting of aryl and heteroaryl;

T⁹⁰⁶ is selected from the group consisting of —C₀₋₆alkyl-S(O)₂—C₀₋₆alkyl-, —C₀₋₆alkyl-C(O)—C₀₋₆alkyl- and C₁₋₃alkyl, wherein T⁹⁰⁶ is substituted at the carbon atom attached to R⁹⁰⁶ with a moiety selected from the group consisting of; aryl, heteroaryl, cycloalkyl and heterocycle;

A⁹⁰⁶ is an optionally substituted unbridged heterocycle;

Q⁹⁰⁶ is a bond;

Het is an optionally substituted 5-membered aryl ring;

L⁹⁰⁶ is a bond or —C₁₋₄alkyl-; and

R^{906a} is —N(R^{906b})OH, wherein R^{906b} is selected from the group consisting of H, optionally substituted alkyl and optionally substituted aryl;

and

provided that Formula (I) excludes those compounds wherein

-Q-J-L-C(O)Z is optionally substituted —C₀-C₄alkyl-X—C₁-C₄alkyl-phenyl-C₂alkenyl-C(O)NHOH;

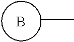

is a 5- or 6-membered aromatic heterocyclic group condensed with a carbon ring or other heterocyclic ring, which

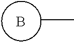

is substituted with 1 to 4 substituents selected from phenyl, another 5- or 6-membered aromatic heterocyclic group and a heterocyclic group, said heterocyclic group being optionally substituted with C₁₋₄alkyl, a benzyl group or a pyridylmethyl group; and

X is a moiety having a structure selected from the group consisting of —C(O)N(R^{A1})—, —O—C(O)—N(R^{A1})—, —SO₂—, —N(R^{A2})SO₂—, wherein R^{A1} and R^{A2} are independently —H or optionally substituted C₁-C₄alkyl;

and

provided that Formula (I) excludes compounds wherein B-Q- is

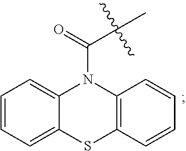 [SMILES: *C(=O)N1c2ccccc2Sc2ccccc21]

and

-J-L- is

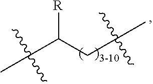

wherein R is directly attached or attached through a linker, and is selected from the group consisting of substituted or unsubstituted aryl, cycloalkyl, cycloalkylamino, naphtha, pyridineamino, piperidino, 9-purine-6-amine, thiazoleamino group, hydroxyl, branched or unbranched alkyl, alkenyl, alkyoxy, aryloxy, arylalkyloxy and pyridine group, wherein the linker is selected from the group consisting of an amide moiety, —O—, —S—, —NH— and —CH₂—; and

provided that Formula (I) excludes compounds of Formula (B)

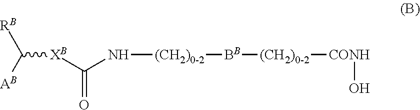

wherein

R^{B} is H or phenyl;

A^{B} is a bi- or tricyclic residue optionally partially or totally unsaturated, and which optionally contains one or more heteroatoms selected from the group consisting of N, S and O, and optionally substituted by hydroxy, alkanoyloxy, primary, secondary or tertiary amino, aminoC₁-C₄alkyl, mono- or di(C₁-C₄)alkyl-aminoC₁-C₄alkyl, halogen, C₁-C₄alkyl and tri(C₁-C₄)alkylammoniumC₁-C₄alkyl;

[symbol] is a chain of 1 to 5 carbon atoms optionally containing a double bond or an NR group, wherein R is H or C₁-C₄alkyl;

X^{B} is absent, an oxygen atom or an NR group, wherein R is H or C₁-C₄alkyl; and

B^{B} is a phenylene or cyclohexylene ring;

and

provided that Formula (I) excludes compounds of Formula (D)

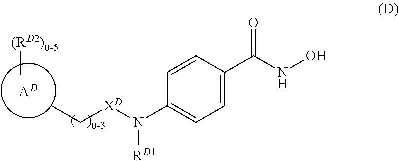

wherein

A^{D} is selected from the group consisting of a 4- to 10-membered aromatic or non-aromatic heterocyclyl;

X^{D} is C═O or S(O)₂;

R^{D1} is H or C₁-C₆alkyl;

R^{D2} is independently selected from the group consisting of oxo, (C═O)—NH₂, C₁-C₆alkyl-aryl and heterocyclyl, when A^{D} is a non-aromatic heterocycle, wherein said alkyl, and aryl moieties are optionally substituted with one to three R^{b}; or

R^{D2} is independently selected from the group consisting of OH, NO₂, (C═O)₀₋₁—O₀₋₁—C₁-C₆alkyl, CN, (C═O)₀₋₁—O₀₋₁—C₃-C₁₀cycloakyl, halogen, (C═O)₀₋₁—N(R^{a})₂, CF₃, NH—S(O)₀₋₂—R^{a}, (C═O)₀₋₁—O₀₋₁-heterocyclyl, (C═O)₀₋₁—O₀₋₁-aryl, S(O)₀₋₂—R^{a}, NH(C═O)R^{a}, C₁-C₆alkyl-aryl and heterocyclyl, when A^{D} is an aromatic heterocyclyl, wherein said alkyl, cycloalkyl, aryl and heterocyclyl are optionally substituted with one to three R^{b};

R^{a} is independently H or C₁-C₆alkyl; and

R^{b} is independently selected from the group consisting of oxo, NO₂, N(R^{a})₂, OH, CN, halogen, CF₃ and C₁-C₆alkyl;

and

provided that Formula (I) excludes compounds of Formula (E)

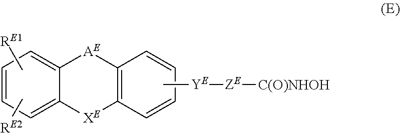

wherein

A^{E} is selected from the group consisting of —CH₂—O—, —CH₂—S—, —CH₂—CH₂— and —NH—CO—;

X^{E} is selected from the group consisting of —N(R^{E3})—, ═C(O) and —CH(OH)—;

Y^{E} is selected from the group consisting of O, S and —N(R^{E4})—;

Z^{E} is selected from the group consisting of a straight chain C4-C8alkylene, wherein one CH₂ group may be replaced by an oxygen or a sulfur atom, or wherein 2 carbon atoms form a C═C double bond, and which is either unsubstituted or substituted by one or two substituents selected from C₁-C₄alkyl and halogen;

R^{E1} and R^{E2} are independently selected from the group consisting of H, halogen, C₁-C₄alkyl, trifluoromethyl, hydroxy, C₁-C₄alkoxy, benzyloxy, C₁-C₃alkylenedioxy, nitro, amino, C₁-C₄alkylamino, di[(C₁-C₄)alkyl]-amino, and C₁-C₄alkanoylamino; and

R^{E3} and R^{E4} are independently selected from H and C₁-C₄alkyl; and

provided that Formula (I) excludes compounds of Formula (F)

A^{F}-Q^{1F}-J^{F}-Q^{2F}-C(O)—NH—OH (F)

wherein

A^{F} is a C₅-C₂₀ aryl group or a 5-20 membered heteroaryl group, each having one ring or two or more fused rings, wherein at least one ring is aromatic, said ary and heteroaryl groups being optionally substituted;

Q^{1F} is a linker group having a backbone length of at least 2 carbon atoms, the linker being optionally substituted;

J^{F} is —N(R^{F})—C(O)— or —C(O)—N(R^{F})—;

Q^{2F} is selected from the group consisting of C₁-C₁₀alkyl, C₅-C₂₀aryl, 5 to 20 membered heteroaryl, C₅-C₂₀aryl-C₁-C₁₀alkyl, 5 to 20 membered heteroaryl-C₁-C₁₀alkyl, C₁-C₁₀alkyl-C₅-C₂₀aryl and C₁-C₁₀alkyl-5 to 20 membered heteroaryl, each of which is optionally substituted; and

R^{F} is selected from the group consisting of H, C₁-C₇alkyl, C₃-C₂₀heterocyclyl and C₅-C₂₀aryl, each of which is optionally substituted; and

provided that Formula (I) excludes compounds wherein

Z is —N(R¹)(OR²);

R¹ and R² are independently selected from the group consisting of H, C₁-C₆alkyl, aryl and heteroaryl;

L is a bond; and

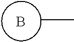

is selected from the group consisting of hydrogen, aryl, aryl-alkyl-, heteroaryl, heteroaryl-alkyl-, heterocyclyl, cycloalkyl, heterocyclyl-alkyl, cycloalkyl-alkyl, C₁-C₁₀alkyl, (aryl)₂-CH—C₀-C₆alkyl-, (aryl)(heteroaryl)CH—C₀-C₆alkyl- and (heteroaryl)₂CH—C₀-C₆alkyl-, each of which is optionally substituted; and

Q comprises a ring selected from the group consisting of

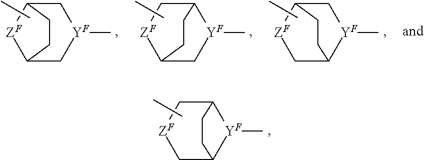

wherein Y^{F} is nitrogen or —CH<, and Z^{F} is oxygen, NH or —CH₂— if Z^{F} is not bonded to

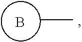

or Z^{F} is nitrogen or —CH< if Z^{F} is bonded to

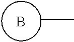

through a covalent bond or a radical group selected from the group consisting of H, —C(R¹)(R²)—, —C₀-C₈alkyl-C(O)—C₀-C₃alkyl-, —C₁-C₈alkyl-, —C₀-C₈alkyl-N(R³)—C(O)—C₀-C₃alkyl-, —C(R¹)(R²)—N(R³)—C(O)—C₀-C₃alkyl-, —C(R¹)(R²)—C(O)—C₀-C₃alkyl-, —C₀-C₈alkyl-O—C(O)—C₀-C₃alkyl-, —C(R¹)(R²)—O—C(O)—C₀-C₃alkyl-, —C₀-C₈alkyl-N(R³)—C(S)—C₀-C₃alkyl-, —C₀-C₈alkyl-O—C(S)—C₀-C₃alkyl-, —C₀-C₈alkyl-N(R³)—S(O)₂—C₀-C₃alkyl-, —C₀-C₈alkyl-heterocyclyl-C₀-C₃alkyl-, a covalent bond, (R³)(R^{3a})N—C₂-C₄alkyl-, —O—C₂-C₄alkyl-, and R³—O—C₂-C₄alkyl-;

or

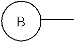

is selected from the group consisting of b-53, b-62 (wherein D³ is

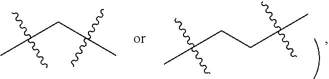

b-69 (wherein R⁴ is H), b-70, b-72 (wherein D³ is

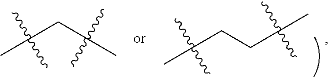

b-92 and b-93; and

Q-J is selected from the group consisting of —X^{F}—C₀₋₄-alkyl-aryl-C₀₋₄-alkyl-, —X^{F}—C₀₋₄alkyl-heteroaryl-C₀₋₄alkyl-, and —X^{F}—C₀₋₄alkyl-heterocyclyl-C₀₋₄alkyl-, wherein said alkyl, aryl, heteroaryl, and heterocyclyl are optionally substituted, and wherein said heterocyclyl is a mono- or bi-saturated or mono- or bi-unsaturated heterocyclic ring, and wherein

X^{F} is selected from the group consisting of

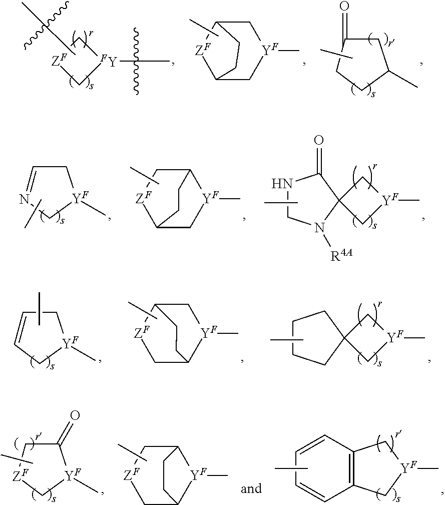

wherein the left side attaches to

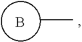

and wherein r and s are each independently 0, 1, 2, 3, 4 or 5, wherein r and s cannot be both 0 and when r or s are 0 then a direct bound in intended; each r′ is independently 0, 1, 3, 3 or 4 and r′ cannot be 0 when s is 0; R^{4A} is H, C₁₋₆alkyl or phenyl; Y^{F} is nitrogen or —CH<, and Z^{F} is oxygen, NH or —CH₂— if Z^{F} is not bonded to

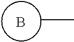

or Z^{F} is nitrogen or —CH< if Z^{F} is bonded to

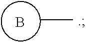

and

provided that Formula (I) excludes those compounds having the following structure:

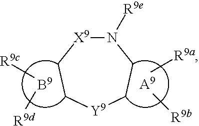

wherein

X⁹ is selected from the group consisting of CO, SO₂ and CH₂;

Y⁹ is selected from the group consisting of N—R^{9f}, CH—OR^{9f}, CH—NR^{9f}R⁹ⁱ and C═CH—CO—R^{9g};

A⁹ and B⁹ are independently selected from 5- or 6-membered rings;

R^{9a}, R^{9b}, R^{9c} and R^{9d} are independently selected from the group consisting of H, halogen, CF₃, NO₂, NR⁹ⁱR^{9j}, CN, COOH, (CH₂)₀₋₂—CONR⁹ⁱR^{9j}, C₁₋₆alkyl, OH, O—C₁₋₆alkyl, O-cyclopropyl, O—(CH₂)₂—O—C₁₋₆alkyl, O—(CH₂)₂—NR⁹ⁱR^{9j}, O—CONHR⁹ⁱ, CH₂—Z⁹—R^{9h}, COR⁹ⁱ, CR⁹ⁱR^{9m}R⁹ⁿ, SR⁹ⁱ, SO₂R^{9o}, CR⁹ⁱNOR⁹ⁱ, CR⁹ⁱNNR⁹ⁱR^{9j}, a Q⁹-(CH₂)₂₋₉CONHOH group, furan, thiophene, pyrrole, oxazole, thiazole, imidazole, pyrazole, isoxazole, isothiazole, 1,2,3-oxathiazole, 1,2,3-triazole, pyridine, pyridazine, pyrimidine, pyrazine, morpholine thiomorpholine, piperidine and pyrrolidine;

R^{9e} and R^{9f} are Q^{9a}-(CH₂)₂₋₉CONHOH;

R^{9g} is NH—(CH₂)₂₋₉CONHOH;

R^{9h} is a (CH₂)P—R^{9k} group, wherein R^{9k} can be methyl or hydroxyl;

Z⁹ is selected from the group consisting of O, NR^{9L} and S;

Q⁹ is selected from the group consisting of a chemical bond, —O—, —S—, —NR^{9L}—, —NR⁹ⁱCO—, —CONR⁹ⁱ—, —W⁹—, —COW⁹—, wherein W⁹ is piperidine or pyrrolidine;

Q^{9a} is a bond or a —CO—;

R⁹ⁱ and R^{9j} are independently H or a C₁₋₆alkyl;

R^{9L} is H or R^{9h};

R^{9m} and R⁹ⁿ can either be a fluorine atom or oxygen atoms linked together by an alkyl chain consisting of 2 or 3 CH₂; and

R^{9o} is a C₁₋₆alkyl; provided that (1) only one (CH₂)₂₋₉CONHOH is present in the molecule and (2) when X⁹ is CO and A⁹ and B⁹ are both benzene then R^{9c} and R^{9d} cannot signify Q⁹-(CH₂)₂₋₉CONHOH.

In a preferred embodiment of the present disclosure,

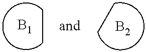

are independently selected from the group consisting of phenyl, heteroaryl and heterocyclyl, wherein each phenyl, heteroaryl and heterocyclyl is optionally substituted with one to three substituents independently selected from the group consisting of halo, —CF₃, —OCF₃, —NO₂, —CN, —C₁-C₆alkyl, —C₁-C₆alkoxyl, —O—C₂-C₆alkyl-O—R⁵³, —O—R⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-SO)₀₋₂NR⁵⁰R⁵¹, C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-C₃-C₇cycloalkyl, —C₀-C₆alkyl-heterocyclyl, —C₀-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³

In a preferred embodiment of the present disclosure, and

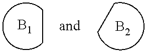

are independently selected from the group consisting of phenyl, heteroaryl and heterocyclyl, wherein each phenyl, heteroaryl and heterocyclyl is optionally substituted with one to three substituents independently selected from the group consisting of R⁴.

In a preferred embodiment of the FTLD targeted agents of the present disclosure, J-Q is selected from the group consisting of —C₁-C₉alkyl, —C₁-C₉heteroalkyl, phenyl, aryl, heteroaryl, —C₁-C₄alkyl-phenyl, —C₁-C₄alkyl-aryl, —C₁-C₄alkyl-heteroaryl, —NR³³aryl, —NR³³—C₁-C₄alkyl-aryl, —NR³³heteroaryl and NR³³—C₁-C₄alkyl-heteroaryl, wherein each alkyl and heteroalkyl is optionally substituted with one or three substituents independently selected from the group consisting of F, —OH and oxo, and wherein each phenyl, aryl and heteroaryl is optionally substituted with one or two substituents independently selected from the group consisting of halo, —OH, —OR⁵³, —C₁-C₄alkyl, —C₁-C₄alkoxyl, —O—C₂-C₄alkyl-O—C₁-C₆alkyl, —CN, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂, wherein R³³ is independently selected from the group consisting of —H, —C₁-C₆alkyl, —C₀-C₆alkyl-C₃-C₇cycloalkyl and —C₀-C₄alkyl-phenyl, wherein each phenyl and cycloalkyl is optionally substituted with one or three substituents independently selected from the group consisting of halo, —OH, —NO₂, —CF₃, —OCF₃, amino, —N(C₁-C₆alkyl)₂, —C₁-C₆alkyl-S(O)₀₋₂R⁵³, —C₁-C₄alkoxyl-CN, —O—C₂alkyl-O—CH₃, —NR⁵⁰R⁵¹, —C₁-C₆alkyl-NR⁵⁰R⁵¹ or —C₁-C₄alkyl.

In a preferred embodiment, embodiment A, of the FTLD targeted agents of the present disclosure, Q comprises a bridged heterocycle,

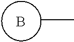

comprises a first ring structure, said first ring structure attached via a covalent bond to said bridged heterocycle and J comprises a second ring structure, said second ring structure attached via a covalent bond to said bridged heterocycle, each of which is optionally substituted. In another preferred embodiment, L is a covalent bond.

In another preferred embodiment, embodiment B, of the FTLD targeted agents according to the present disclosure, L is a covalent bond, Q is a heterocycle comprising a one or three carbon bridge, and J is heteroaryl, wherein each of

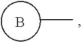

Q and J are optionally substituted.

In another preferred embodiment, embodiment B-2, of the FTLD targeted agents according to the present disclosure, L is a covalent bond, Q comprises a heterocycle comprising an unsubstituted methylene, ethylene or propylene bridge, and J is heteroaryl, wherein each of

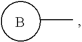

Q and J are otherwise optionally substituted.

In another preferred embodiment, embodiment B-3, of the FTLD targeted agents according to the present disclosure, L is a covalent bond, Q comprises a heterocycle comprising an unsubstituted methylene, ethylene or propylene bridge, and J is aryl, wherein each of

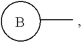

Q and J are otherwise optionally substituted.

In another preferred embodiment, embodiment C, of the FTLD targeted agents according to the present disclosure, L is a covalent bond, Q is a heterocycle comprising a one or three carbon bridge, and J is pyrimidine, wherein each of

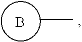

Q and J are optionally substituted.

In another preferred embodiment, embodiment D, of the FTLD targeted agents according to the present disclosure, L is a covalent bond, Q is a heterocycle comprising an unsubstituted methylene bridge, and J is pyrimidine, wherein each of

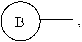

Q and J are otherwise optionally substituted.

In another preferred embodiment, embodiment E, of the FTLD targeted agents according to the present disclosure, L is a covalent bond, Q is a heterocycle comprising a three carbon bridge; and J is pyrimidine, wherein each of

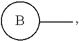

Q and J are optionally substituted.

In another preferred embodiment, embodiment F, of the compounds according to the present disclosure, L is a covalent bond, Q is a 2,5-diazabicyclo[2.2.1]heptane, and J is pyrimidine, wherein each of

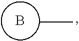

Q and J are optionally substituted.

In a preferred embodiment, embodiment G, of each of the foregoing,

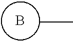

is an optionally substituted aryl or heteroary, preferably aryl, more preferably phenyl.

In another preferred embodiment, embodiment G-1, of each of the embodiments A to F,

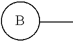

is an optionally substituted heteroary, preferably pyridine.
In a preferred embodiment, embodiment H, of the FTLD targeted agents of the present disclosure,

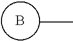

is a radical selected from the group consisting of

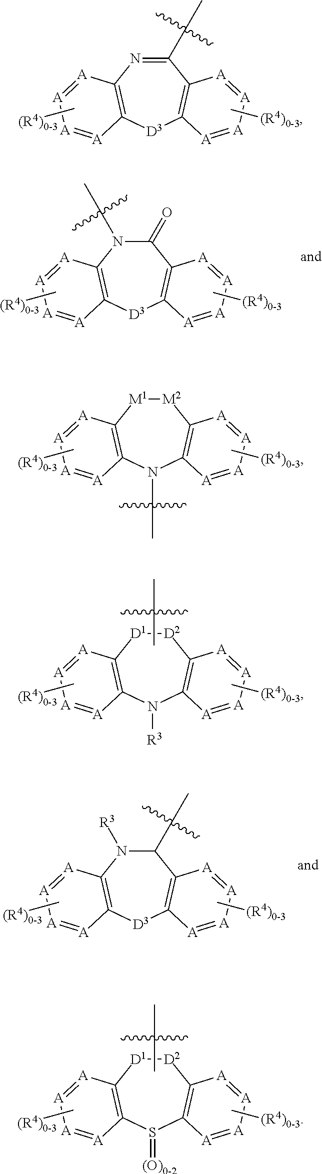

In another preferred embodiment, embodiment I, of the FTLD targeted agents according to the present disclosure,

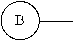

is a radical selected from the group consisting of

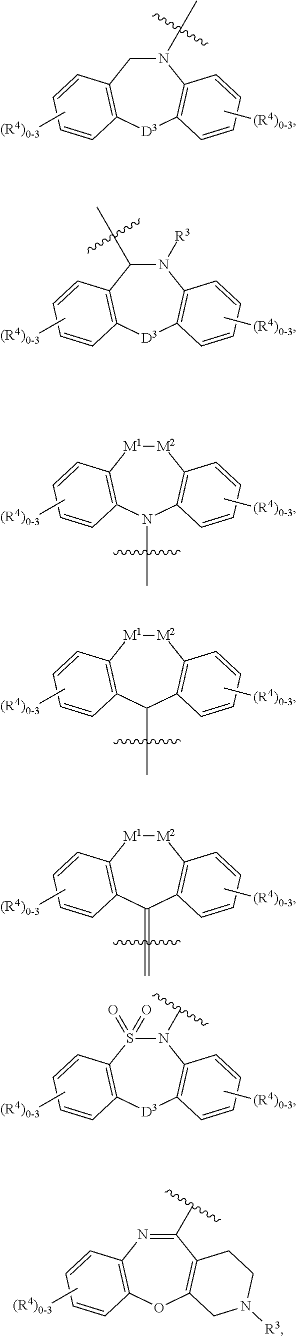

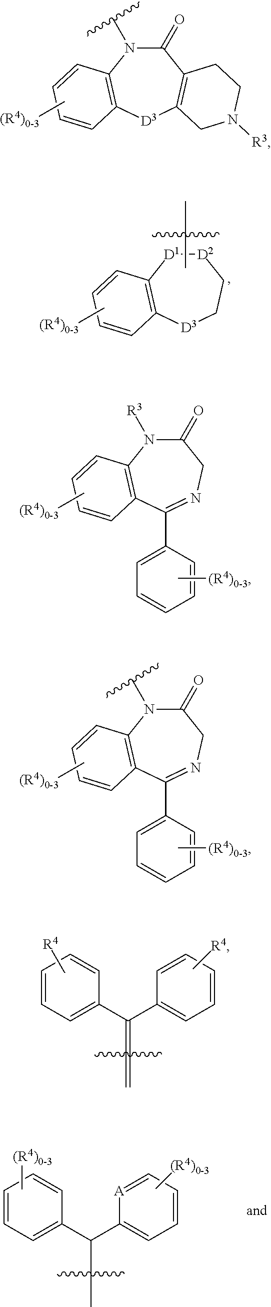

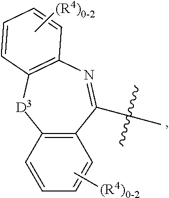

wherein when

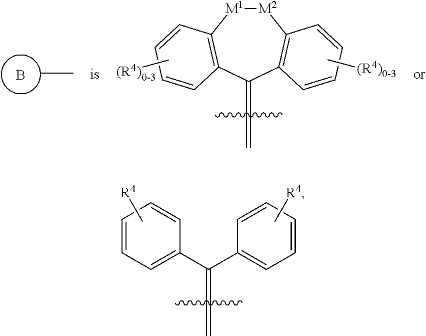

Q is attached via

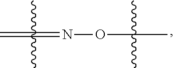 [SMILES: *ON=*]

and wherein when

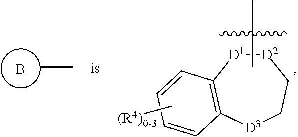

Q is attached via D¹-D².
In another preferred embodiment, embodiment J, of the FTLD targeted agents according to the present disclosure

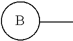

is a radical selected from the group consisting of

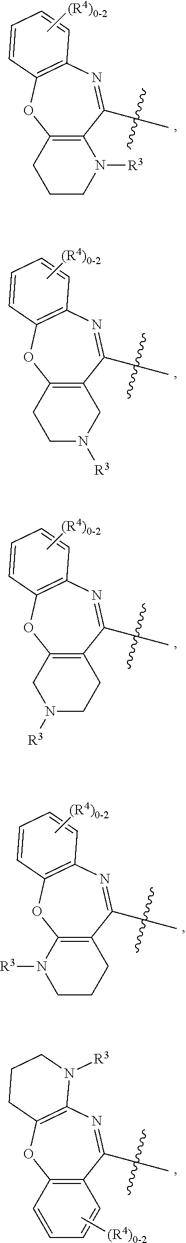

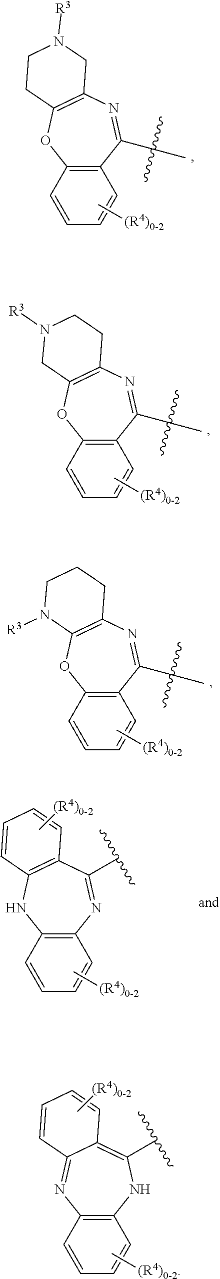

In another preferred embodiment, embodiment K, of the FTLD targeted agents according to the present disclosure, Q is an optionally substituted moiety selected from the group consisting of

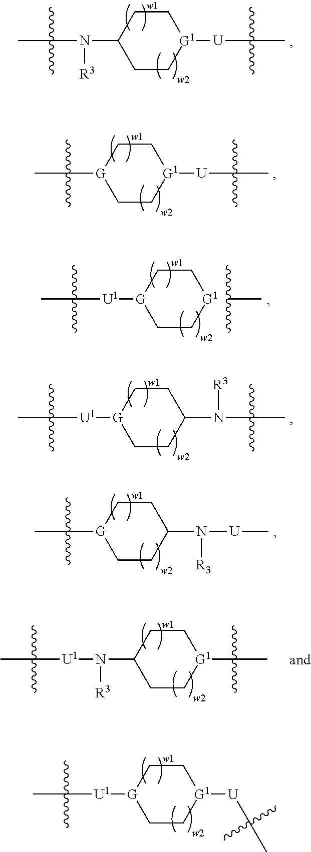

or where possible (R,R) or (S,S) enantiomer or a mixture of enantiomers, preferably an (R,R) enantiomer, more preferably an (S,S) enantiomer thereof, wherein G and G¹ are independently selected from —CH— and N; w1 and w2 are independently 0, 1, 2 or 3, provided that structure includes a 0 (i.e., a bond), 1, 2 or 3 carbon bridge between two non-adjacent carbon atoms, provided that

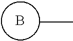

is absent when U¹ is H, N(R³)(R^{3a})—C₂-C₄alkyl- or R³—O—C₂-C₄alkyl-. Preferrably the ring size is 6, 7, 8 or 9 ring atoms, excluding any bridge atoms.
In another preferred embodiment, embodiment L, of the FTLD targeted agents according to the present disclosure, Q is an optionally substituted moiety selected from the group consisting of

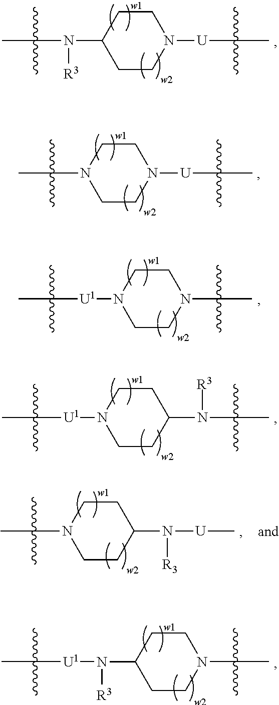

or where possible, an (R,R) or (S,S) enantiomer or a mixture of enantiomers, preferably an (R,R) enantiomer, more preferably an (S,S) enantiomer thereof, wherein w1 and w2 are independently 0, 1, 2 or 3, provided that when the ring includes two N atoms, then w1 and w2 are independently 1, 2 or 3; and wherein each ring structure includes a 0 (i.e., a bond), 1, 2 or 3 carbon bridge between two non-adjacent carbon atoms, provided that

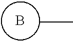

is absent when U¹ is H, N(R³)(R^{3a})—C₂-C₄alkyl- or R³—O—C₂-C₄alkyl-.

In another preferred embodiment, embodiment M, of the FTLD targeted agents according to the present disclosure, Q is an optionally substituted moiety, selected from the group consisting of

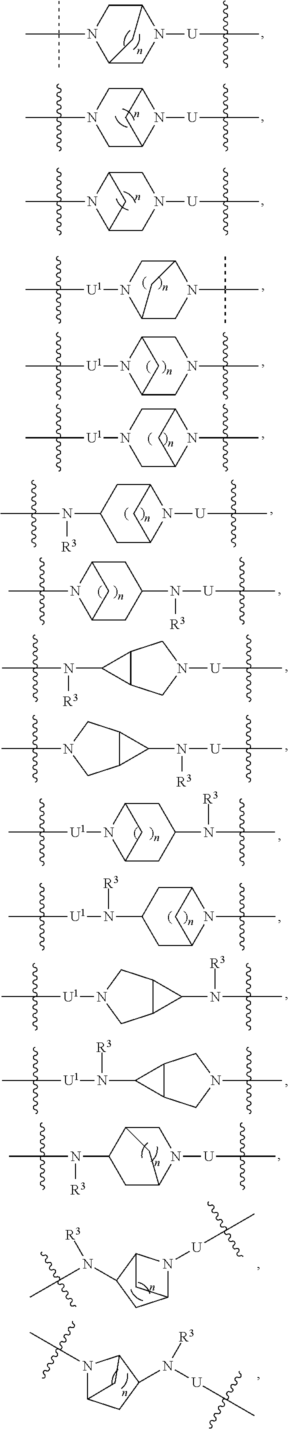

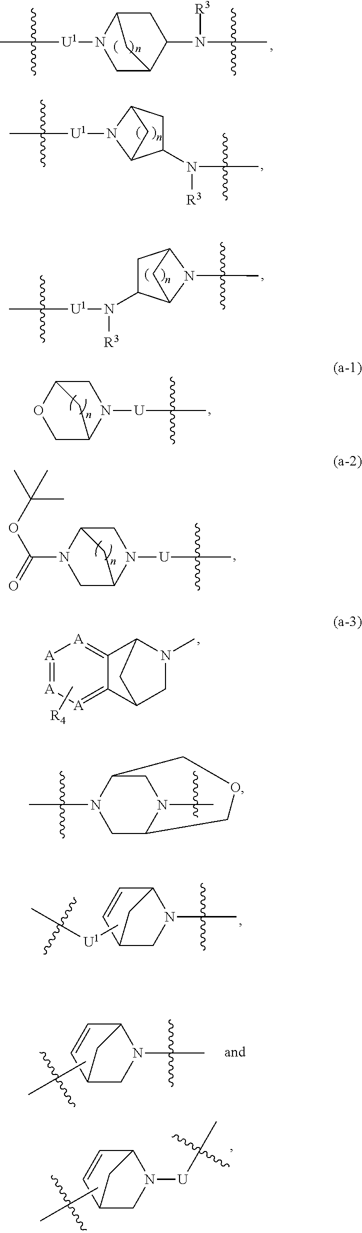

or wherein possible, a (R,R) or (S,S) enantiomer or a mixture of enantiomers, preferably an (R,R) enantiomer, more preferably an (S,S) enantiomer thereof, wherein n is 1, 2 or 3, and

wherein

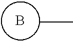

is absent when Q is structure (a-1), (a-2), (a-3) or when U¹ is H, N(R³)(R^{3a})—C₂-C₄alkyl- or R³—O—C₂-C₄alkyl-.
In another preferred embodiment, embodiment N, of the FTLD targeted agents according to the present disclosure, Q is an optionally substituted moiety selected from the group consisting of

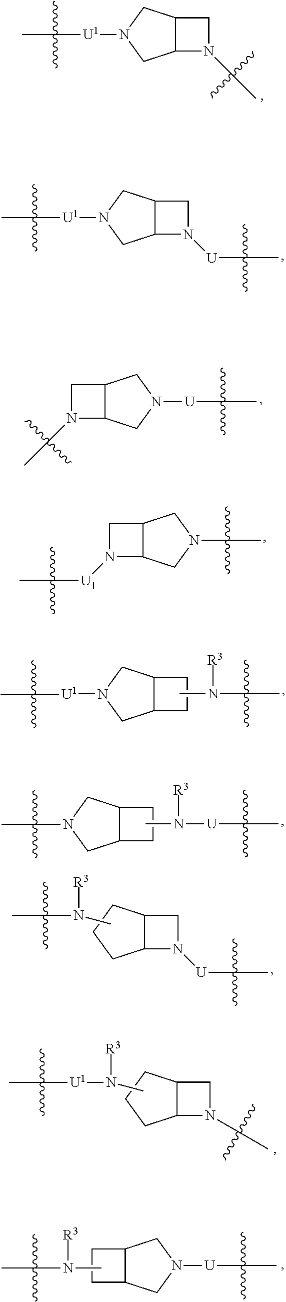

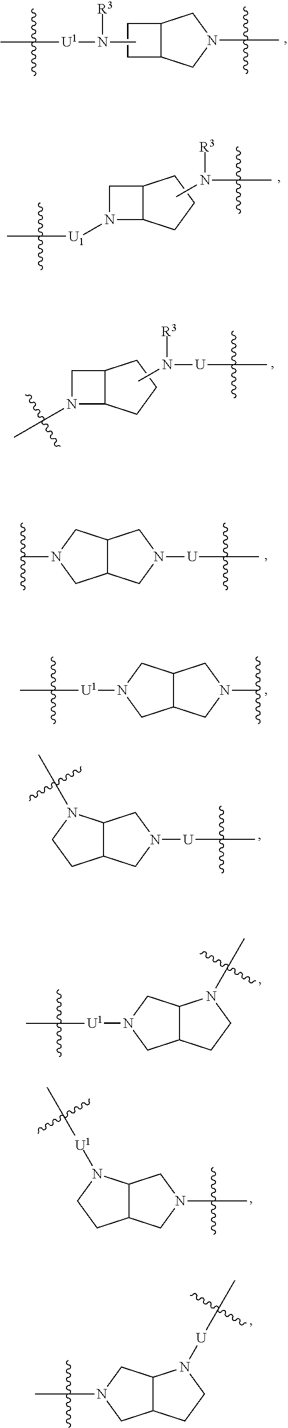

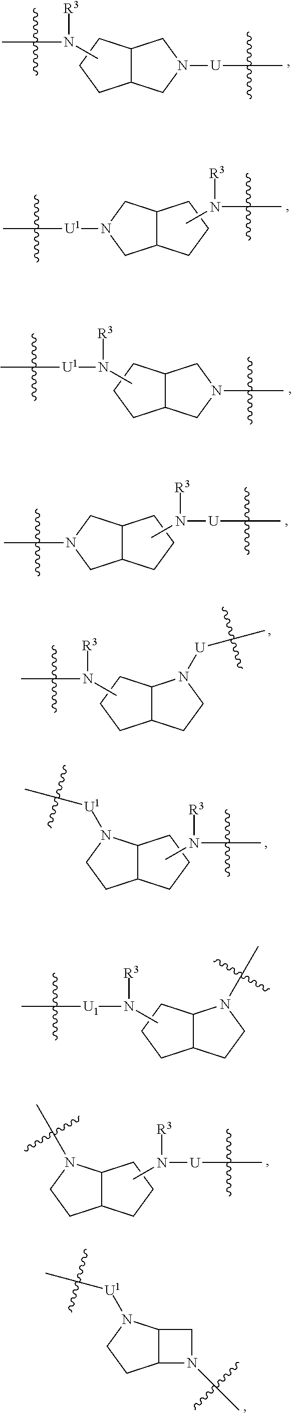

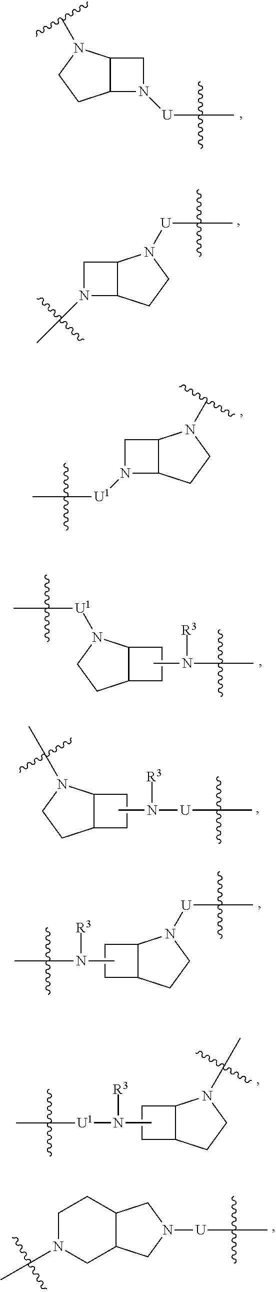

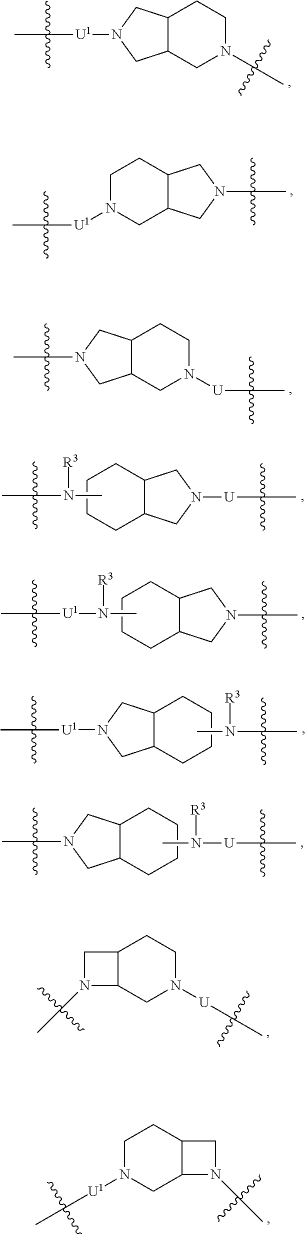

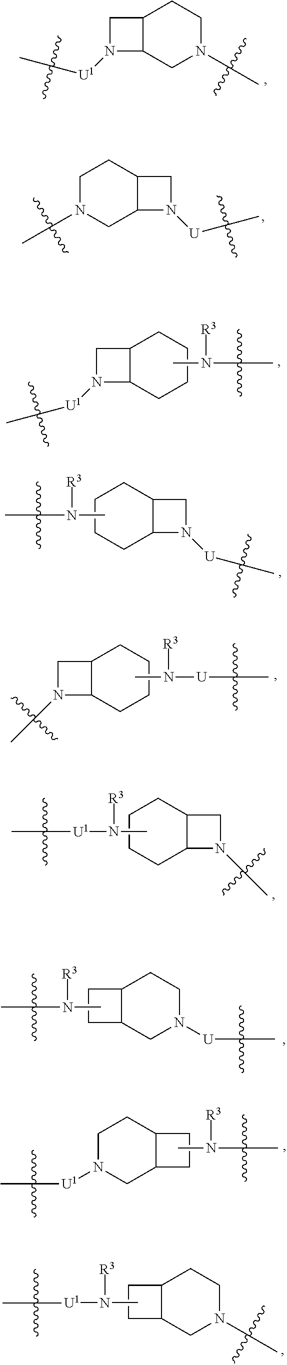

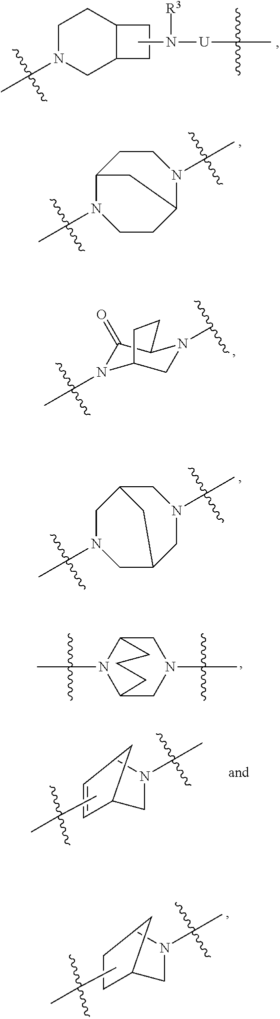

or wherein possible, a (R,R) or (S,S) enantiomer or a mixture of enantiomers, preferably an (R,R) enantiomer, more preferably an (S,S) enantiomer thereof, wherein

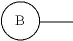

is absent when U¹ is H, N(R³)(R^{3a})—C₂-C₄alkyl- or R³—O—C₂-C₄alkyl-.

In a preferred embodiment, embodiment O, of the FTLD targeted agents of the present disclosure,

Z is —N(R¹)(OR²);

L is a covalent bond;

J is selected from the group consisting of a covalent bond, ═CH—, —C₁-C₈alkyl-, —C₀-C₃alkyl-C₁-C₈heteroalkyl-C₀-C₃alkyl-, —C₀-C₃alkyl-C₂-C₈alkenyl-C₀-C₃alkyl-, —C₀-C₃alkyl-C₂-C₈alkynyl-C₀-C₃alkyl-, —C₀-C₆alkyl-aryl-C₀-C₆alkyl-, —C₀-C₆alkyl-aryl-C₂-C₆heteroalkyl-, —C₀-C₆alkyl-cycloalkyl-C₀-C₆alkyl-, —C₄-C₆heterocyclyl-aryl-C₀-C₆alkyl-, —C₄-C₆heterocyclyl-aryl-C₀-C₆heteroalkyl-, —C₀-C₆alkyl-C₄-C₆heterocyclyl-C₀-C₆alkyl-, —C₀-C₆alkyl-heteroaryl-C₀-C₆alkyl-, —C₀-C₆alkyl-heteroaryl-C₀-C₆heteroalkyl-, —C₄-C₆heterocyclyl-heteroaryl-C₀-C₆alkyl-, —C₀-C₆alkyl-aryl-C₂-C₆alkynyl-, —C₀-C₆alkyl-heteroaryl-C₂-C₆alkynyl-, —C₀-C₆alkyl-aryl-C₂-C₆alkynyl-C₂-C₆alkenyl-, —C₀-C₆alkyl-aryl-C₂-C₆alkenyl-, —C₀-C₆alkyl-heteroaryl-C₂-C₆alkenyl-, —C₂-C₆alkenyl-aryl-C₀-C₆alkyl-, —C₂-C₆alkenyl-heteroaryl-C₀-C₆alkyl-, —C₀-C₆alkylaryl-aryl-C₀-C₆alkyl-, —C₀-C₆alkylaryl-heteroaryl-C₀-C₆alkyl- and —C₀-C₆alkyl-C₃-C₆cycloalkyl-C₀-C₆alkyl-, wherein each alkyl, alkenyl, alkynyl, heteroalkyl, aryl, heteroaryl, heterocyclyl, and cycloalkyl moiety is optionally substituted, wherein when J is ═CH—, Q is a covalent bond and B is attached through a carbon sp² to J;

Q is a moiety selected from the group consisting of

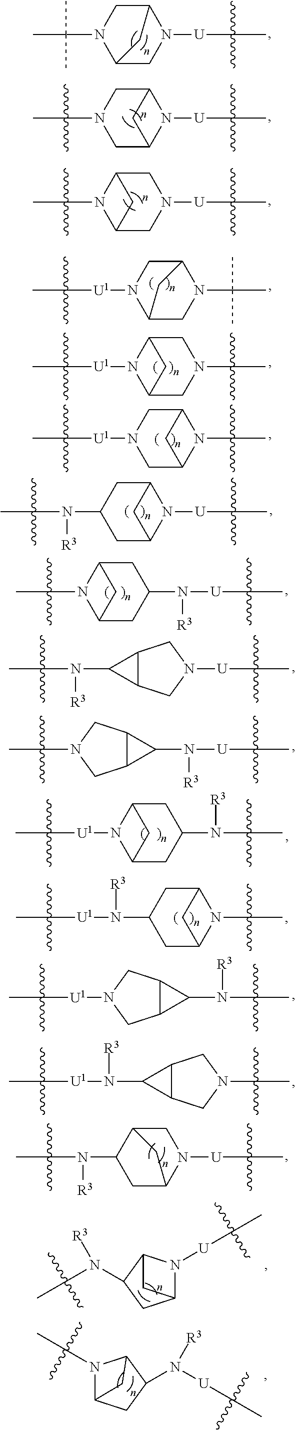

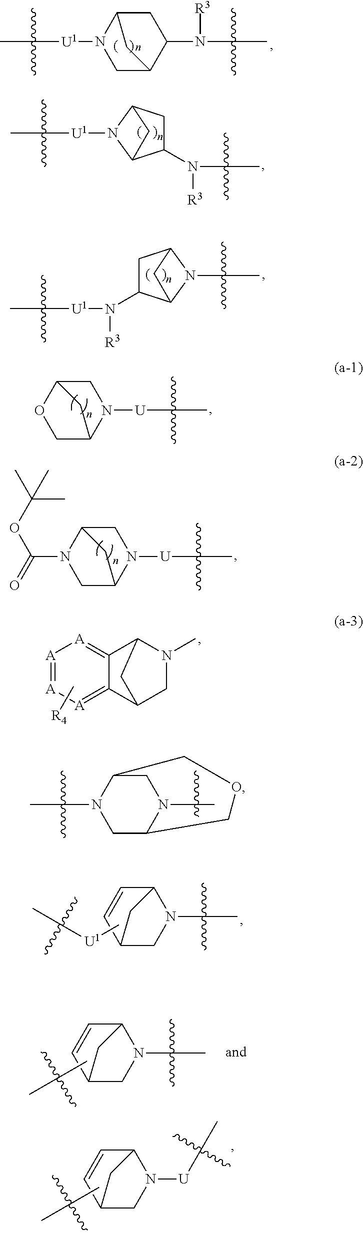

or an optionally substituted (R,R) or (S,S) enantiomer or a mixture of enantiomers, preferably an (R,R) enantiomer, more preferably an (S,S) enantiomer thereof, wherein n is 0, 1, 2 or 3; and

U is selected from the group consisting of —C₀-C₈alkyl-C(O)—C₀-C₃alkyl-, —C₁-C₈alkyl-, —C₀-C₈alkyl-N(R³)—C(O)—C₀-C₃alkyl-, —C₀-C₈alkyl-O—C(O)—C₀-C₃alkyl-, —C₀-C₈alkyl-N(R³)—C(S)—C₀-C₃alkyl-, —C₀-C₈alkyl-O—C(S)—C₀-C₃alkyl-, —C₀-C₈alkyl-N(R³)—S(O)₂—C₀-C₃alkyl-, —C₀-C₈alkyl-heterocyclyl-C₀-C₃alkyl-, a covalent bond and —O—C₂-C₄alkyl-; and

U¹ is selected from the group consisting of H, —C₀-C₈alkyl-C(O)—C₀-C₃alkyl-, —C₁-C₈alkyl-, —C₀-C₈alkyl-N(R³)—C(O)—C₀-C₃alkyl-, —C₀-C₈alkyl-O—C(O)—C₀-C₃alkyl-, —C₀-C₈alkyl-N(R³)—C(S)—C₀-C₃alkyl-, —C₀-C₈alkyl-O—C(S)—C₀-C₃alkyl-, —C₀-C₈alkyl-N(R³)—S(O)₂—C₀-C₃alkyl-, —C₀-C₈alkyl-heterocyclyl-C₀-C₃alkyl-, a covalent bond, (R³)(R^{3a})N—C₂-C₄alkyl-, —O—C₂-C₄alkyl-, and R³—O—C₂-C₄alkyl-;

wherein

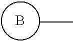

is absent when Q is structure (a-1), (a-2), (a-3) or when U¹ is H, N(R³)(R^{3a})—C₂-C₄alkyl- or R³—O—C₂-C₄alkyl-.

In a preferred embodiment of embodiment O, embodiment O-1, of the FTLD targeted agents according to the present disclosure, J is selected from the group consisting of a —C₀-C₃alkyl-C₁-C₈heteroalkyl-C₀-C₃alkyl-, —C₀-C₆alkyl-aryl-C₀-C₆alkyl-, —C₀-C₆alkyl-aryl-C₂-C₆heteroalkyl-, —C₀-C₆alkyl-cycloalkyl-C₀-C₆alkyl-, —C₄-C₆heterocyclyl-aryl-C₀-C₆alkyl-, —C₄-C₆heterocyclyl-aryl-C₀-C₆heteroalkyl-, —C₀-C₆alkyl-C₄-C₆heterocyclyl-C₀-C₆alkyl-, —C₀-C₆alkyl-heteroaryl-C₀-C₆alkyl-, —C₀-C₆alkyl-heteroaryl-C₀-C₆heteroalkyl-, —C₄-C₆heterocyclyl-heteroaryl-C₀-C₆alkyl-, —C₀-C₆alkyl-aryl-C₂-C₆alkynyl-, —C₀-C₆alkyl-heteroaryl-C₂-C₆alkynyl-, —C₀-C₆alkyl-aryl-C₂-C₆alkynyl-C₂-C₆alkenyl-, —C₀-C₆alkyl-aryl-C₂-C₆alkenyl-, —C₀-C₆alkyl-heteroaryl-C₂-C₆alkenyl-, —C₂-C₆alkenyl-aryl-C₀-C₆alkyl-, —C₂-C₆alkenyl-heteroaryl-C₀-C₆alkyl-, —C₀-C₆alkylaryl-aryl-C₀-C₆alkyl-, —C₀-C₆alkylaryl-heteroaryl-C₀-C₆alkyl- and —C₀-C₆alkyl-C₃-C₆cycloalkyl-C₀-C₆alkyl-, wherein each alkyl, alkenyl, alkynyl, heteroalkyl, aryl, heteroaryl, heterocyclyl, and cycloalkyl moiety is optionally substituted.

In a preferred embodiment of embodiment 0-1, embodiment 0-2, J is —C₀-C₆alkyl-heteroaryl-C₀-C₆alkyl- or —C₀-C₆alkyl-aryl-C₀-C₆alkyl-.

In a preferred embodiment of embodiment 0-2, embodiment 0-3, Q is selected from the group consisting of

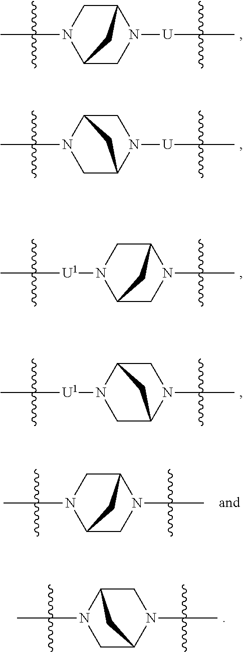

In a preferred embodiment of embodiment 0-3, embodiment 0-4, U and U¹ are a covalent bond.

In a preferred embodiment of embodiment 0-3 embodiment 0-5, U and U¹ are —C(O)—.

In another preferred embodiment of embodiment 0-3, embodiment 0-6, moiety U is —C(O)—O—C₀-C₃alkyl-.

In another preferred embodiment of embodiment 0-3, embodiment 0-7, U¹ is —C₀-C₃alkyl-O—C(O)—.

In another preferred embodiment, embodiment P of the FTLD targeted agents according to the present disclosure

J is selected from the group consisting of —C₁-C₈alkyl-, —C₀-C₆alkyl-aryl-C₀-C₃alkyl-C₂alkenyl-C₀-C₃alkyl, —C₀-C₆alkyl-heteroaryl-C₀-C₃alkyl-C₂alkenyl-C₀-C₃alkyl, —C₀-C₆alkyl-aryl-C₀-C₆alkyl- and —C₀-C₆alkyl-heteroaryl-C₀-C₆alkyl-, wherein each is optionally substituted;

Q is selected from the group consisting of a covalent bond, —C₁-C₈alkyl-, ═N—O—, —C₀-C₆alkyl-N(R³)—C₀-C₃alkyl-, —C₀-C₆alkyl-heterocyclyl-C₀-C₃alkyl-, —C₀-C₆alkyl-C(O)—C₀-C₃alkyl-, —C₀-C₆alkyl-O—C₀-C₃alkyl-, —C₀-C₆alkyl-(CR³═CR³)₁₋₂—C₀-C₆alkyl-, —C₀-C₆alkyl-(C≡C)₁₋₂—C₀-C₆alkyl-, —C₀-C₆alkyl-N(R³)—C(O)—C₀-C₃alkyl-, wherein each alkyl and heterocyclyl moiety is optionally substituted;

or

Q is selected from the group consisting of:

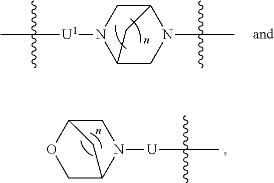

wherein

U¹ is selected from the group consisting of —C₀-C₈alkyl-C(O)—C₀-C₃alkyl-, —C₁-C₈alkyl-, —C₀-C₈alkyl-O—C(O)—C₀-C₃alkyl- and a covalent bond;

wherein, when B is attached to Q via a N in B, then Q is selected from the group consisting of a covalent bond, —C(O)—C₁-C₃alkyl-O—, —C₁-C₈alkyl-, —C₀-C₆alkyl-C(O)—C₀-C₃alkyl-, —C₂-C₆alkyl-O—C₀-C₃alkyl-, —C₁-C₆alkyl-(CR³═CR³)₁₋₂—C₀-C₆alkyl- and —C₁-C₆alkyl-(CC)₁₋₂—C₀-C₆alkyl-, wherein each alkyl moiety is optionally substituted;

provided that

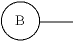

is absent when Q is

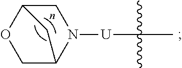

and

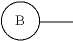

is selected from the group consisting of hydrogen, aryl, cycloalkyl, heterocyclyl, heteroaryl, heteroarylalkyl, aryl-alkyl-, (heteroaryl)₂-CH—C₀-C₆alkyl- and (aryl)₂-CH—C₀-C₆alkyl-, each of which is optionally substituted, provided that Q is

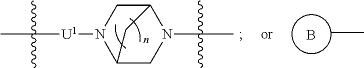

is a radical selected from the group consisting of

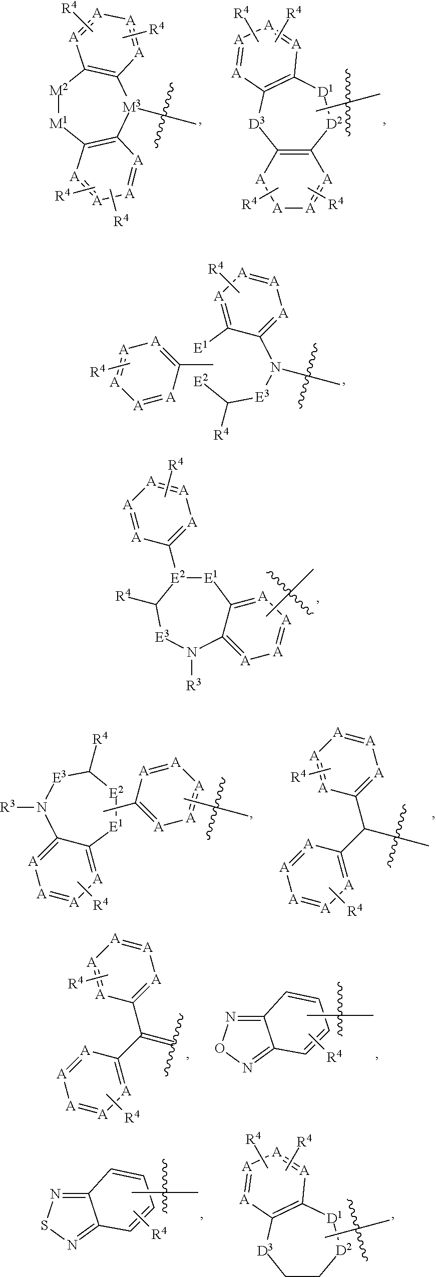

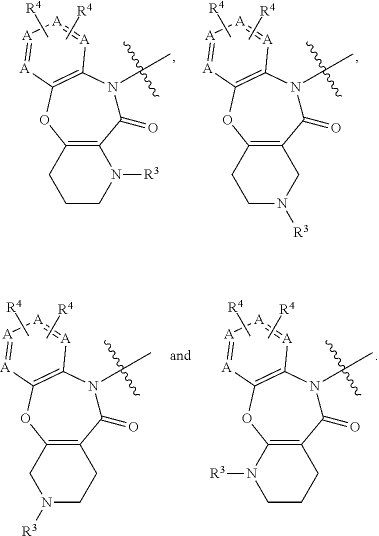

In a preferred embodiment P, embodiment P-1,

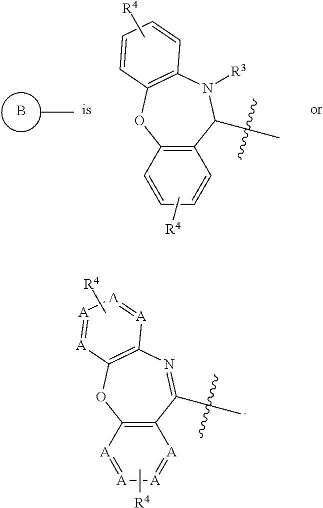

In another preferred embodiment, embodiment Q, of the FTLD targeted agents according to the present disclosure, the compound has a structure selected from the group consisting of

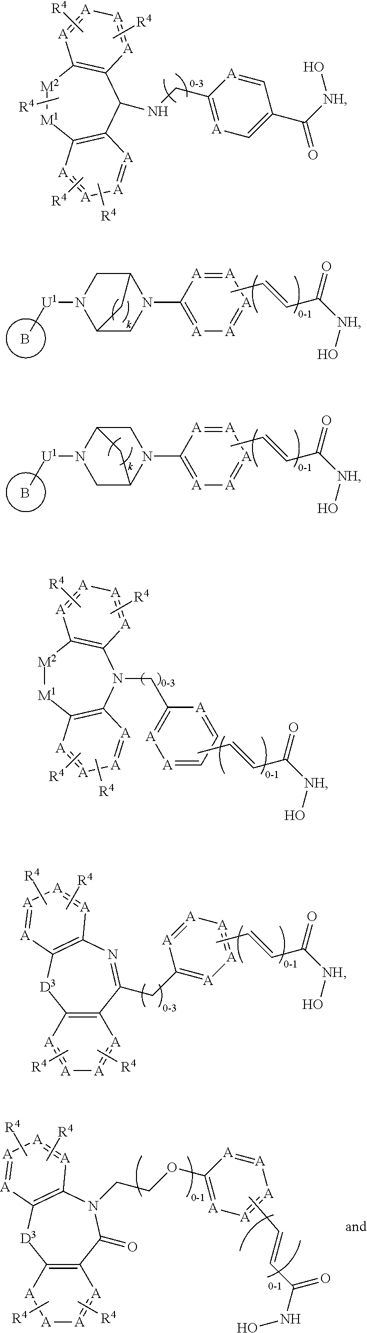

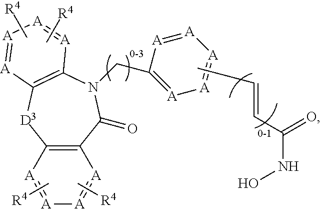

wherein k is 0 or 3.

In another preferred embodiment, embodiment R, of the FTLD targeted agents according to the present disclosure, Z is —NR¹OR², R¹ and R² are H, and L is a covalent bond.

In another preferred embodiment, embodiment S, of the FTLD targeted agents according to the present disclosure, Z is H and L is —N(OH).

In another preferred embodiment, embodiment T, of the FTLD targeted agents according to the present disclosure, J is selected from the group consisting of —C₁-C₈alkyl-, —C₀-C₃alkyl-C₁-C₈alkenyl-C₀-C₃-alkyl, —C₀-C₆alkyl-aryl-C₀-C₆alkyl-, —C₀-C₆alkyl-aryl-C₂-C₆alkenyl, —C₀-C₆alkyl-heteroaryl-C₀-C₆alkyl- and —C₀-C₆alkyl-heterocyclyl-heteroaryl-C₀-C₆alkyl-.

In another preferred embodiment, embodiment U, of the FTLD targeted agents according to the present disclosure, J is selected from the group consisting of

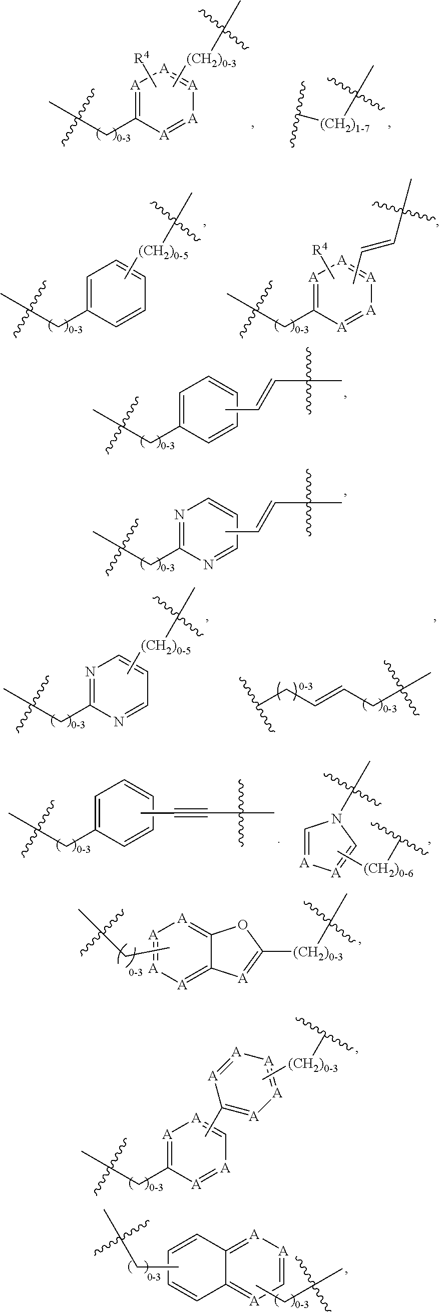

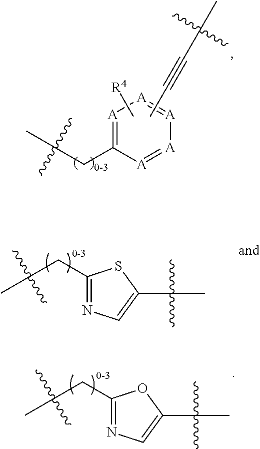

In another preferred embodiment, embodiment V, of the FTLD targeted agents according to the present disclosure, Q is selected from the group consisting of a covalent bond, —C₁-C₈alkyl-, ═N—O—, —C₀-C₆alkyl-N(R³)—C₀-C₃alkyl-, —C₀-C₆alkyl-heterocyclyl-C₀-C₃alkyl-, —C₀-C₆alkyl-C(O)—C₀-C₃alkyl-, —C₀-C₆alkyl-O—C₀-C₃alkyl-, —C₀-C₆alkyl-(CR³═CR³)₁₋₂—C₀-C₆alkyl-, —C₀-C₆alkyl-(C≡C)₁₋₂—C₀-C₆alkyl-, —C₀-C₆alkyl-N(R³)—C(O)—C₀-C₃alkyl-, —C₀-C₆alkyl-N(R³)—C(O)-alkenyl-C₀-C₄alkyl-, —C₀-C₆alkyl-C(O)—N(R³)—C₀-C₄alkyl-, —C₀-C₆alkyl-SO₂—N(R³)—C₀-C₃alkyl-, —C₀-C₆alkyl-N(R³)—SO₂—C₀-C₃alkyl-, —C₀-C₃alkyl-N(R³)—S(O)₂—N(R³)—C₀-C₃alkyl-, —C₀-C₆alkyl-S—C₀-C₃alkyl-, —C₀-C₆alkyl-S(O)—C₀-C₃alkyl-, —C₀-C₆alkyl-S(O)₂—C₀-C₃alkyl-, —C₀-C₆alkyl-N(R³)—C(O)—N(R³)—C₀-C₃alkyl-, —C₀-C₃alkyl-C═N—O—C₀-C₃alkyl-, -heterocyclyl-C₀-C₃alkyl-heterocyclyl-C₀-C₃alkyl-, —SO₂—C₀-C₆alkyl-heterocyclyl-C₀-C₃alkyl-, —C(O)—C₀-C₆alkyl-bridged heterocyclyl-C₀-C₃alkyl-, —N(R³)—C(O)—C₀-C₆alkyl-heterocyclyl-C₀-C₃alkyl-, —O—C(O)—C₀-C₆alkyl-heterocyclyl-C₀-C₃alkyl-, —N(R³)—C(S)—C₀-C₆alkyl-heterocyclyl-C₀-C₃alkyl-, —O—C(S)—C₀-C₆alkyl-heterocyclyl-C₀-C₃alkyl-, —N(R³)—S(O)₂—C₀-C₆alkyl-heterocyclyl-C₀-C₃alkyl-, —C₀-C₆alkyl-heterocyclyl-C₀-C₃alkyl-SO₂—N(R³)—, —C₀-C₆ alkyl-heterocyclyl-C₀-C₃alkyl-C(O)—N(R³)— and —C₀-C₆alkyl-heterocyclyl-C₀-C₃alkyl-C(O)—O—, wherein each alkyl, heterocyclyl and alkenyl moiety is optionally substituted.

In another preferred embodiment, embodiment W, of the FTLD targeted agents according to the present disclosure, Q is selected from the group consisting of covalent bond, ═N—O—, —C₁-C₈alkyl-, —C₀-C₆ alkyl-N(R₃)—C₀-C₃ alkyl-, —C₀-C₆ alkyl-C(O)—C₀-C₃ alkyl-, —C₀-C₆ alkyl-C(O)NR₃—C₀-C₃ alkyl-, —C₀-C₆ alkyl-O—C₀-C₃ alkyl- and —C₀-C₃alkyl-heterocyclyl-C₀-C₃-alkyl.

In another preferred embodiment, embodiment X, of the FTLD targeted agents according to the present disclosure, Q is selected from the group consisting of

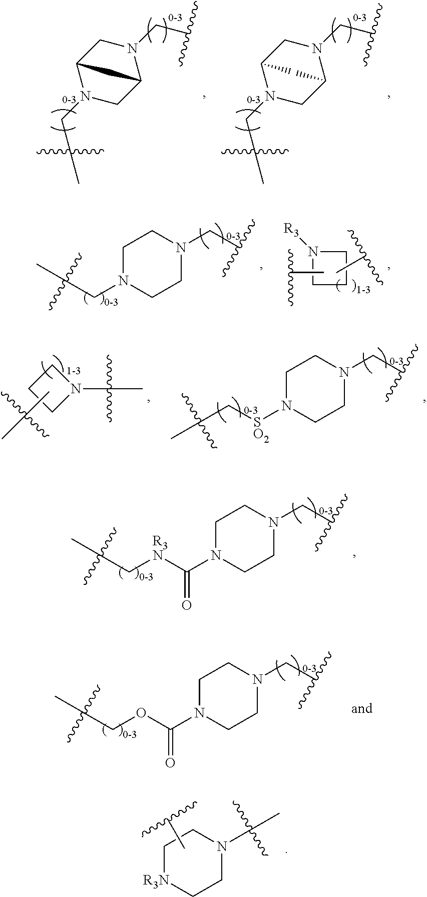

In another preferred embodiment, embodiment Y, of the FTLD targeted agents according to the present disclosure,

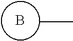

is selected from the group consisting of aryl, aryl-alkyl-, heteroaryl, heteroaryl-alkyl-, (aryl)₂-CH—C₀-C₆alkyl-, (aryl)(heteroaryl)CH—C₀-C₆alkyl-, (heteroaryl)₂CH—C₀-C₆alkyl- and (aryl)₂-CH—C₀-C₆alkyl-C(O)—, —wherein each group is optionally substituted with 1, 2, 3 or 4 substituents independently selected from the group consisting of hydroxy, amino, halo, C₁-C₆alkyl, nitro, cyano, C₂-C₆alkoxy, C₁-C₆alkylamino and CF₃.
In another preferred embodiment, embodiment Z, of the FTLD targeted agents according to the present disclosure,

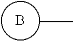

is selected from the group consisting of

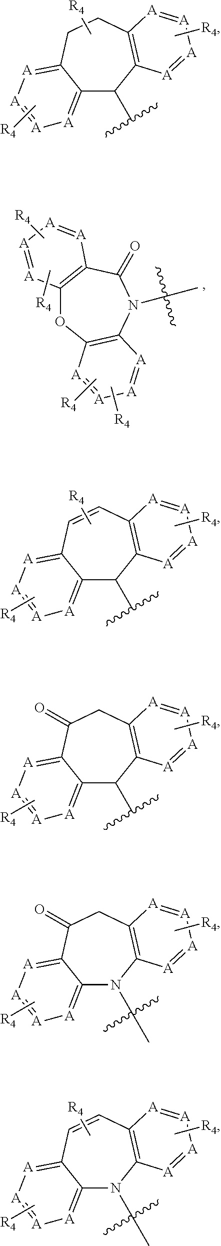

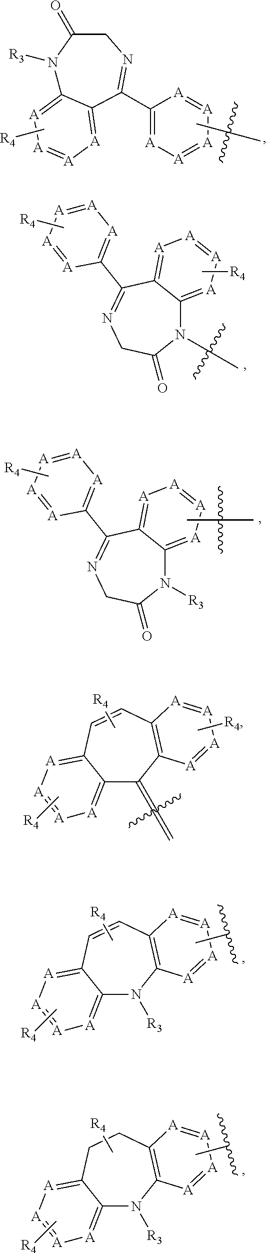

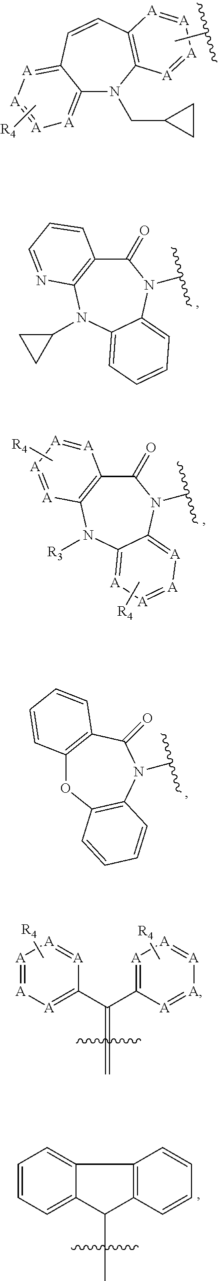

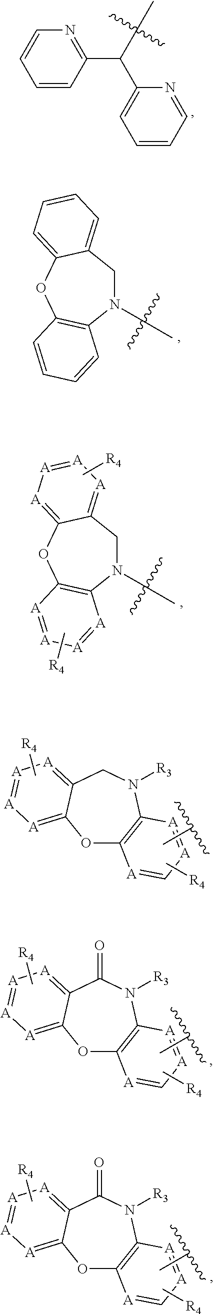

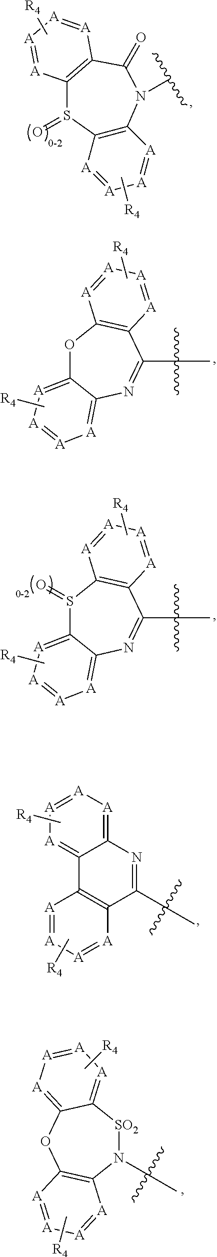

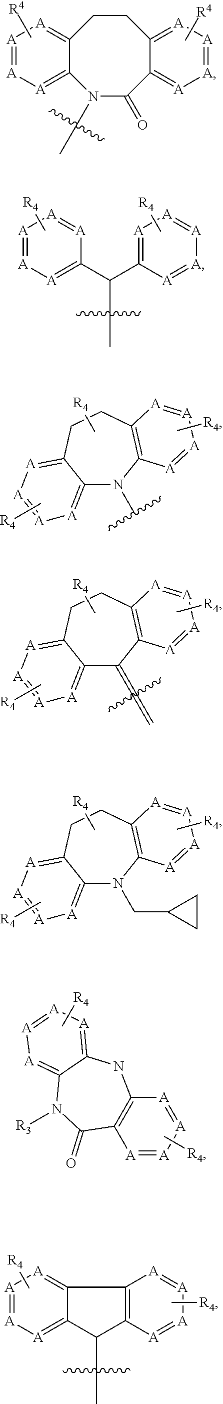

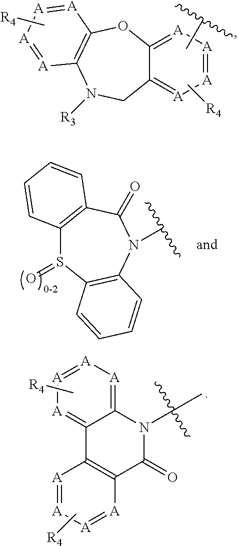

In another preferred embodiment, embodiment AA, of the FTLD targeted agents according to the present disclosure, each alkyl, alkenyl, alkynyl, heteroalkyl, aryl, heteroaryl, heterocyclyl, and cycloalkyl moiety of J is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl.

In another preferred embodiment, embodiment BB, of the FTLD targeted agents according to the present disclosure, Q is selected from the group consisting of a covalent bond, —C₁-C₈alkyl-, ═N—O—, —C₀-C₆alkyl-N(R³)—C₀-C₃alkyl-, —C₀-C₆alkyl-heterocyclyl-C₀-C₃alkyl-, —C₀-C₆alkyl-C(O)—C₀-C₃alkyl-, —C₀-C₆alkyl-O—C₀-C₃alkyl-, —C₀-C₆alkyl-(CR³═CR³)₁₋₂—C₀-C₆alkyl-, —C₀-C₆alkyl-(C≡C)₁₋₂—C₀-C₆alkyl-, —C₀-C₆alkyl-N(R³)—C(O)—C₀-C₃alkyl-, —C₀-C₆alkyl-N(R³)—C(O)-alkenyl-C₀-C₄alkyl-, —C₀-C₆alkyl-C(O)—N(R³)—C₀-C₄alkyl-, —C₀-C₆alkyl-SO₂—N(R³)—C₀-C₃alkyl-, —C₀-C₆alkyl-N(R³)—SO₂—C₀-C₃alkyl-, —C₀-C₃alkyl-N(R³)—S(O)₂—N(R³)—C₀-C₃alkyl-, —C₀-C₆alkyl-S—C₀-C₃alkyl-, —C₀-C₆alkyl-S(O)—C₀-C₃alkyl-, —C₀-C₆alkyl-S(O)₂—C₀-C₃alkyl-, —C₀-C₆alkyl-N(R³)—C(O)—N(R³)—C₀-C₃alkyl-, —C₀-C₃alkyl-C═N—O—C₀-C₃alkyl-, -heterocyclyl-C₀-C₃alkyl-heterocyclyl-C₀-C₃alkyl-, —SO₂—C₀-C₆alkyl-heterocyclyl-C₀-C₃alkyl-, —C(O)—C₀-C₆alkyl-bridged heterocyclyl-C₀-C₃alkyl-, —N(R³)—C(O)—C₀-C₆alkyl-heterocyclyl-C₀-C₃alkyl-, —O—C(O)—C₀-C₆alkyl-heterocyclyl-C₀-C₃alkyl-, —N(R³)—C(S)—C₀-C₆alkyl-heterocyclyl-C₀-C₃alkyl-, —O—C(S)—C₀-C₆alkyl-heterocyclyl-C₀-C₃alkyl-, —N(R³)—S(O)₂—C₀-C₆alkyl-heterocyclyl-C₀-C₃alkyl-, —C₀-C₆alkyl-heterocyclyl-C₀-C₃alkyl-SO₂—N(R³)—, —C₀-C₆ alkyl-heterocyclyl-C₀-C₃alkyl-C(O)—N(R³)— and —C₀-C₆alkyl-heterocyclyl-C₀-C₃alkyl-C(O)—O—, wherein each alkyl, heterocyclyl and alkenyl moiety is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl.

In another preferred embodiment, embodiment CC, of the FTLD targeted agents according to the present disclosure, Q is an optionally substituted (1R,4R) or (1S,4S) 2,5-diazabicyclo[2.2.1]heptane enantiomer or a mixture of enantiomers, preferably an (1R,4R) enantiomer, more preferably an (1S,4S) enantiomer, selected from the group consisting of

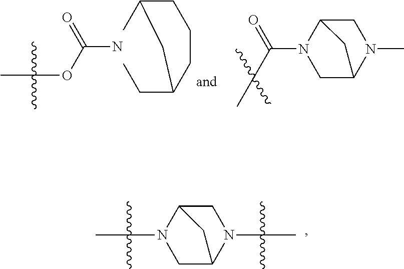

or

Q is

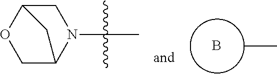

is absent; or

Q is

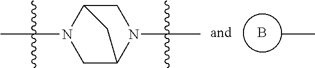

### is H.

In another preferred embodiment, embodiment DD, of the FTLD targeted agents according to the present disclosure, when

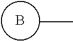

is attached to Q via a N in

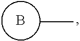

then Q is selected from the group consisting of —C₁-C₈alkyl-, —C₂-C₆alkyl-N(R³)—C₀-C₃alkyl-, —C₀-C₆alkyl-heterocyclyl-C₀-C₃alkyl-, —C₀-C₆alkyl-C(O)—C₀-C₃alkyl-, —C₂-C₆alkyl-O—C₀-C₃alkyl-, —C₁-C₆alkyl-(CR³═CR³)₁₋₂—C₀-C₆alkyl-, —C₁-C₆alkyl-(C≡C)₁₋₂—C₀-C₆alkyl-, —C₂-C₆alkyl-N(R³)—C(O)—C₀-C₃alkyl, —C₂-C₆alkyl-N(R³)—C(O)-alkenyl-C₀-C₃alkyl, —C₀-C₆alkyl-C(O)—N(R³)—C₀-C₄alkyl-, —C(O)—O—C₀-C₄alkyl, —C₀-C₆alkyl-S(O)₂—N(R³)—C₀-C₃alkyl, —C₂-C₆alkyl-N(R³)—S(O)₂—C₀-C₃alkyl, —C₂-C₃alkyl-N(R³)—S(O)₂—N(R³)—C₀-C₃alkyl-, —C₂-C₆alkyl-S—C₀-C₃alkyl, —C₂-C₆alkyl-S(O)—C₀-C₃alkyl, —C₀-C₆alkyl-S(O)₂—C₀-C₃alkyl, —C₂-C₆alkyl-N(R³)—C(O)—N(R³)—C₀-C₃alkyl, —C₂-C₃alkyl-C═N—O—C₀-C₃alkyl, —SO₂—C₀-C₆alkyl-heterocyclyl-C₀-C₃alkyl-, —C(O)—C₀-C₆alkyl-heterocyclyl-C₀-C₃alkyl-, —N(R³)—C(O)—C₀-C₆alkyl-heterocyclyl-C₀-C₃alkyl-, —O—C(O)—C₀-C₆alkyl-heterocyclyl-C₀-C₃alkyl-, —N(R³)—C(S)—C₀-C₆alkyl-heterocyclyl-C₀-C₃alkyl-, —O—C(S)—C₀-C₆alkyl-heterocyclyl-C₀-C₃alkyl-, —N(R³)—S(O)₂—C₀-C₆alkyl-heterocyclyl-C₀-C₃alkyl-, —C₀-C₆alkyl-heterocyclyl-C₀-C₃alkyl-S(O₂)—N(R³)—, —C₀-C₆alkyl-heterocyclyl-C₀-C₃alkyl-C(O)—N(R³)— and —C₀-C₆alkyl-heterocyclyl-C₀-C₃alkyl-C(O)—O—, wherein each alkyl, heterocyclyl and alkenyl moiety is optionally substituted with from one to three substituents independently selected the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl, and wherein the heterocyclyl moiety optionally has a bridge of —(CH₂)₀₋₃—.

In another preferred embodiment, embodiment EE, of the FTLD targeted agents according to the present disclosure, each R₃ is independently selected from the group consisting of —H, alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl, heteroaryl and a covalent bond, wherein each alkyl, alkenyl, alkynyl, heteroalkyl, cycloalkyl, heterocyclyl, aryl and heteroaryl moiety is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl.

In another preferred embodiment, embodiment FF, of the FTLD targeted agents according to the present disclosure, Q-J-L is selected from the group consisting of —C₃-C₈alkyl-, —C(O)—C₃-C₈alkyl-, —C₀-C₃alkyl-O—C₃-C₈alkyl-, —C₀-C₃alkyl-C₁-C₄alkenyl-C₀-C₃alkyl-, ═N—O—C₁-C₈alkyl-, ═N—O—C₀-C₃alkyl-aryl-C₀-C₃alkyl-, ═N—O—C₀-C₃alkyl-aryl-C₀-C₃alkenyl-, ═N—O—C₀-C₃alkyl-aryl-C₀-C₃alkynyl-, ═N—O—C₀-C₃alkyl-heteroaryl-C₀-C₃alkyl-, ═N—O—C₀-C₃alkyl-heteroaryl-C₀-C₃alkenyl-, ═N—O—C₀-C₃alkyl-heteroaryl-C₀-C₃alkynyl-, —C₀-C₃alkyl-aryl-, —C₀-C₃alkyl-aryl-C₀-C₃alkyl-, —C₀-C₃alkyl-aryl-C₂-C₄alkenyl-, —C₀-C₃alkyl-aryl-C₂-C₄alkynyl-, —C₀-C₃alkyl-heteroaryl-C₀-C₃alkyl-, —C₁-C₃alkyl-heteroaryl-C₁-C₃alkenyl-, —C₁-C₃alkyl-heteroaryl-C₁-C₃alkynyl-, —C₀-C₃alkyl-N(R³)—C₀-C₃alkyl-aryl-C₀-C₃alkyl-, —C₀-C₃alkyl-N(R³)—C₀-C₃alkyl-aryl-C₂-C₃alkenyl-, —C₀-C₃alkyl-N(R³)—C₀-C₃alkyl-aryl-C₂-C₃alkynyl-, —C₀-C₃alkyl-N(R³)—C₀-C₃alkyl-heteroaryl-C₀-C₃alkyl-, —C₀-C₃alkyl-N(R³)—C₀-C₃alkyl-heteroaryl-C₂-C₃alkenyl-, —C₀-C₃alkyl-N(R³)—C₀-C₃alkyl-heteroaryl-C₂-C₃alkynyl-, —C₀-C₃alkyl-C(O)—N(R³)—C₀-C₃alkyl-aryl-C₀-C₃alkyl-, —C₀-C₃alkyl-C(O)—N(R³)—C₀-C₃alkyl-aryl-C₂-C₃alkenyl-, —C₀-C₃alkyl-C(O)—N(R³)— C₀-C₃alkyl-aryl-C₂-C₃alkynyl-, —C₀-C₃alkyl-C(O)—N(R³)—C₀-C₃alkyl-heteroaryl-C₀-C₃alkyl-, —C₀-C₃alkyl-C(O)—N(R³)—C₀-C₃alkyl-heteroaryl-C₂-C₃alkenyl-, —C₀-C₃alkyl-C(O)—N(R³)—C₀-C₃alkyl-heteroaryl-C₂-C₃alkynyl-, —C₀-C₃alkyl-heterocyclyl-C₀-C₃alkyl-aryl-, —C₀-C₃alkyl-heterocyclyl-C₀-C₃alkyl-aryl-C₀-C₃alkyl-, —C₀-C₃alkyl-heterocyclyl-C₀-C₃alkyl-aryl-C₂-C₄alkenyl, —C₀-C₃alkyl-heterocyclyl-C₀-C₃alkyl-aryl-C₂-C₄alkynyl, —C₀-C₃alkyl-heterocyclyl-C₀-C₃alkyl-heteroaryl-C₀-C₃alkyl, —C₀-C₃alkyl-heterocyclyl-C₁-C₃alkyl-heteroaryl-C₂-C₃alkenyl-, —C₀-C₃alkyl-heterocyclyl-C₁-C₃alkyl-heteroaryl-C₂-C₃alkynyl-, —C₂-C₄alkyl-O—C₀-C₃alkyl-aryl-, —C₂-C₄alkyl-O—C₀-C₃alkyl-aryl-C₀-C₃alkyl-, —C₂-C₄alkyl-O—C₀-C₃alkyl-aryl-C₂-C₄alkenyl, —C₂-C₄alkyl-O—C₀-C₃alkyl-aryl-C₂-C₄alkynyl, —C₂-C₄alkyl-O—C₀-C₃alkyl-heteroaryl-C₀-C₃alkyl, —C₂-C₄alkyl-O—C₁-C₃alkyl-heteroaryl-C₂-C₃alkenyl- and —C₂-C₄alkyl-O—C₁-C₃alkyl-heteroaryl-C₂-C₃alkynyl-, wherein each alkyl, alkenyl, aryl, alkynyl, heteroaryl and heterocyclyl moiety is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl.

In another preferred embodiment, embodiment GG, of the FTLD targeted agents according to the present disclosure,

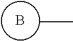

is selected from the group consisting of hydrogen, aryl, aryl-alkyl-, heteroaryl, heteroaryl-alkyl-, (aryl)₂-CH—C₀-C₆alkyl-, (aryl)(heteroaryl)CH—C₀-C₆alkyl-, (heteroaryl)₂CH—C₀-C₆alkyl- and (aryl)₂-CH—C₀-C₆alkyl-C(O)—, each of which is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl, provided that variable n of Q is 0, 1 or 3.

In another preferred embodiment, embodiment HH,

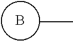

is selected from the group consisting of structures (b-1) to (b-121) and Q-J-L taken together is selected from the group consisting of —C₃-C₈alkyl-, —C(O)—C₃-C₈alkyl-, —C₀-C₃alkyl-O—C₃-C₈alkyl-, —C₀-C₃alkyl-C₁-C₄alkenyl-C₀-C₃alkyl-, ═N—O—C₁-C₈alkyl-, ═N—O—C₀-C₃alkyl-aryl-C₀-C₃alkyl-, ═N—O—C₀-C₃alkyl-aryl-C₀-C₃alkenyl-, ═N—O—C₀-C₃alkyl-aryl-C₀-C₃alkynyl-, ═N—O—C₀-C₃alkyl-heteroaryl-C₀-C₃alkyl-, ═N—O—C₀-C₃alkyl-heteroaryl-C₀-C₃alkenyl-, ═N—O—C₀-C₃alkyl-heteroaryl-C₀-C₃alkynyl-, —C₀-C₃alkyl-aryl-C₀-C₃alkyl-, —C₀-C₃alkyl-aryl-C₂-C₄alkenyl-, —C₀-C₃alkyl-aryl-C₂-C₄alkynyl-, —C₀-C₃alkyl-heteroaryl-C₀-C₃alkyl-, —C₀-C₃alkyl-heteroaryl-C₁-C₃alkenyl-, —C₀-C₃alkyl-heteroaryl-C₁-C₃alkynyl-, —C₀-C₃alkyl-N(R³)—C₀-C₃alkyl-aryl-C₀-C₃alkyl-, —C₀-C₃alkyl-N(R³)—C₀-C₃alkyl-aryl-C₂-C₃alkenyl-, —C₀-C₃alkyl-N(R³)—C₀-C₃alkyl-aryl-C₂-C₃alkynyl-, —C₀-C₃alkyl-N(R³)—C₀-C₃alkyl-heteroaryl-C₀-C₃alkyl-, —C₀-C₃alkyl-N(R³)—C₀-C₃alkyl-heteroaryl-C₂-C₃alkenyl-, —C₀-C₃alkyl-N(R³)—C₀-C₃alkyl-heteroaryl-C₂-C₃alkynyl-, —C₀-C₃alkyl-C(O)—N(R³)— C₀-C₃alkyl-aryl-C₀-C₃alkyl-, —C₀-C₃alkyl-N(R³)—C(O)—C₀-C₃alkyl-aryl-C₀-C₃alkyl-, —C₀-C₃alkyl-C(O)—N(R³)—C₀-C₃alkyl-aryl-C₂-C₃alkenyl-, —C₀-C₃alkyl-N(R³)—C(O)—C₀-C₃alkyl-aryl-C₂-C₃alkenyl-, —C₀-C₃alkyl-C(O)—N(R³)—C₀-C₃alkyl-aryl-C₂-C₃alkynyl-, —C₀-C₃alkyl-N(R³)—C(O)—C₀-C₃alkyl-aryl-C₂-C₃alkynyl-, —C₀-C₃alkyl-C(O)—N(R³)—C₀-C₃alkyl-heteroaryl-C₀-C₃alkyl-, —C₀-C₃alkyl-N(R³)—C(O)—C₀-C₃alkyl-heteroaryl-C₀-C₃alkyl-, —C₀-C₃alkyl-C(O)—N(R³)—C₀-C₃alkyl-heteroaryl-C₂-C₃alkenyl-, —C₀-C₃alkyl-N(R³)—C(O)—C₀-C₃alkyl-heteroaryl-C₂-C₃alkenyl-, —C₀-C₃alkyl-C(O)—N(R³)—C₀-C₃alkyl-heteroaryl-C₂-C₃alkynyl-, —C₀-C₃alkyl-N(R³)—C(O)—C₀-C₃alkyl-heteroaryl-C₂-C₃alkynyl-, —C₀-C₃alkyl-heterocyclyl-C₀-C₃alkyl-aryl-C₀-C₃alkyl-, —C₀-C₃alkyl-C(O)-heterocyclyl-C₀-C₃alkyl-aryl-C₀-C₃alkyl-, —C₀-C₃alkyl-N(R³)—C(O)-heterocyclyl-C₀-C₃alkyl-aryl-C₀-C₃alkyl-, —C₀-C₃alkyl-O—C(O)-heterocyclyl-C₀-C₃alkyl-aryl-C₀-C₃alkyl-, —C₀-C₃alkyl-heterocyclyl-C₀-C₃alkyl-aryl-C₂-C₄alkenyl, —C₀-C₃alkyl-C(O)-heterocyclyl-C₀-C₃alkyl-aryl-C₂-C₄alkenyl, —C₀-C₃alkyl-N(R³)—C(O)-heterocyclyl-C₀-C₃alkyl-aryl-C₂-C₄alkenyl, —C₀-C₃alkyl-O—C(O)-heterocyclyl-C₀-C₃alkyl-aryl-C₂-C₄alkenyl, —C₀-C₃alkyl-heterocyclyl-C₀-C₃alkyl-aryl-C₂-C₄alkynyl, —C₀-C₃alkyl-C(O)-heterocyclyl-C₀-C₃alkyl-aryl-C₂-C₄alkynyl, —C₀-C₃alkyl-N(R³)—C(O)-heterocyclyl-C₀-C₃alkyl-aryl-C₂-C₄alkynyl, —C₀-C₃alkyl-O—C(O)-heterocyclyl-C₀-C₃alkyl-aryl-C₂-C₄alkynyl, —C₀-C₃alkyl-heterocyclyl-C₀-C₃alkyl-heteroaryl-C₀-C₃alkyl, —C₀-C₃alkyl-C(O)-heterocyclyl-C₀-C₃alkyl-heteroaryl-C₀-C₃alkyl, —C₀-C₃alkyl-N(R³)—C(O)-heterocyclyl-C₀-C₃alkyl-heteroaryl-C₀-C₃alkyl, —C₀-C₃alkyl-O—C(O)-heterocyclyl-C₀-C₃alkyl-heteroaryl-C₀-C₃alkyl, —C₀-C₃alkyl-heterocyclyl-C₁-C₃alkyl-heteroaryl-C₂-C₃alkenyl-, —C₀-C₃alkyl-C(O)-heterocyclyl-C₁-C₃alkyl-heteroaryl-C₂-C₃alkenyl-, —C₀-C₃alkyl-N(R³)—C(O)-heterocyclyl-C₁-C₃alkyl-heteroaryl-C₂-C₃alkenyl-, —C₀-C₃alkyl-O—C(O)-heterocyclyl-C₁-C₃alkyl-heteroaryl-C₂-C₃alkenyl-, —C₀-C₃alkyl-heterocyclyl-C₁-C₃alkyl-heteroaryl-C₂-C₃alkynyl-, —C₀-C₃alkyl-C(O)-heterocyclyl-C₁-C₃alkyl-heteroaryl-C₂-C₃alkynyl-, —C₀-C₃alkyl-N(R³)—C(O)-heterocyclyl-C₁-C₃alkyl-heteroaryl-C₂-C₃alkynyl-, —C₀-C₃alkyl-O—C(O)-heterocyclyl-C₁-C₃alkyl-heteroaryl-C₂-C₃alkynyl-, —C₂-C₄alkyl-O—C₀-C₃alkyl-aryl-, —C₂-C₄alkyl-O—C₀-C₃alkyl-aryl-C₀-C₃alkyl-, —C₂-C₄alkyl-O—C₀-C₃alkyl-aryl-C₂-C₄alkenyl, —C₂-C₄alkyl-O—C₀-C₃alkyl-aryl-C₂-C₄alkynyl, —C₂-C₄alkyl-O—C₀-C₃alkyl-heteroaryl-C₀-C₃alkyl, —C₂-C₄alkyl-O—C₁-C₃alkyl-heteroaryl-C₂-C₃alkenyl-, —C₂-C₄alkyl-O—C₁-C₃alkyl-heteroaryl-C₂-C₃alkynyl-,
—C₀-C₆alkyl-U-bridged heterocyclyl-heteroaryl-C₀-C₆alkyl-,
—C₀-C₆alkyl-U-bridged heterocyclyl-N(R³)-heteroaryl-C₀-C₆alkyl-,
—C₀-C₆alkyl-U—N(R)-bridged heterocyclyl-heteroaryl-C₀-C₆alkyl-,
—C₀-C₆alkyl-U-bridged heterocyclyl-aryl-C₀-C₆alkyl-,
—C₀-C₆alkyl-U-bridged heterocyclyl-N(R³)-aryl-C₀-C₆alkyl-,
—C₀-C₆alkyl-U—N(R)-bridged heterocyclyl-aryl-C₀-C₆alkyl-,
—C₀-C₆alkyl-U-bridged heterocyclyl-aryl-C₂-C₆alkenyl-,
—C₀-C₆alkyl-U-bridged heterocyclyl-N(R³)-aryl-C₂-C₆alkenyl-,
—C₀-C₆alkyl-U—N(R)-bridged heterocyclyl-aryl-C₂-C₆alkenyl-,
—C₀-C₆alkyl-U-bridged heterocyclyl-heteroaryl-C₂-C₆alkenyl-,
—C₀-C₆alkyl-U-bridged heterocyclyl-N(R³)-heteroaryl-C₂-C₆alkenyl-,
—C₀-C₆alkyl-U—N(R)-bridged heterocyclyl-heteroaryl-C₂-C₆alkenyl-,
—C₀-C₆alkyl-bridged heterocyclyl-U-heteroaryl-C₀-C₆alkyl-,
—C₀-C₆alkyl-N(R)-bridged heterocyclyl-U-heteroaryl-C₀-C₆alkyl-,
—C₀-C₆alkyl-bridged heterocyclyl-N(R³)—U-heteroaryl-C₀-C₆alkyl-,
—C₀-C₆alkyl-bridged heterocyclyl-U-aryl-C₀-C₆alkyl-,
—C₀-C₆alkyl-N(R)-bridged heterocyclyl-U-aryl-C₀-C₆alkyl-,
—C₀-C₆alkyl-bridged heterocyclyl-N(R³)—U-aryl-C₀-C₆alkyl-,
—C₀-C₆alkyl-bridged heterocyclyl-U-aryl-C₂-C₆alkenyl-,
—C₀-C₆alkyl-N(R³)-bridged heterocyclyl-U-aryl-C₂-C₆alkenyl-,
—C₀-C₆alkyl-bridged heterocyclyl-N(R³)—U-aryl-C₂-C₆alkenyl-,
—C₀-C₆alkyl-bridged heterocyclyl-U-heteroaryl-C₂-C₆alkenyl-,
—C₀-C₆alkyl-N(R³)-bridged heterocyclyl-U-heteroaryl-C₂-C₆alkenyl-, and
—C₀-C₆alkyl-bridged heterocyclyl-N(R³)—U-heteroaryl-C₂-C₆alkenyl-,

wherein each alkyl, alkenyl, aryl, alkynyl, heteroaryl and heterocyclyl moiety is optionally substituted; and wherein the bridge is methylene or propylene.

In another preferred embodiment, embodiment II, of the FTLD targeted agents according to the present disclosure B-Q-J-L- are taken together, wherein each such B-Q-J-L group is optionally substituted with up to 4 substituents independently selected from the group consisting of hydroxy, amino, halo, C₁-C₆alkyl, nitro, cyano, C₂-C₆alkoxy, C₁-C₆-amino and CF₃, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl and alkylheteroaryl.

In another preferred embodiment, embodiment JJ, of the FTLD targeted agents according to the present disclosure, R⁴ is independently selected from the group consisting of —H, C₁-C₆alkyl, C₂-C₆alkenyl, C₂-C₆alkynyl, C₁-C₆alkyl-R³, —C₀-C₆alkyl-OR³, —C₀-C₆alkyl-OR¹, —C₀-C₆alkyl-C(O)—OR³, —C₀-C₆alkyl-C(O)NR³R^{3a}, —CH═CH—C(O)—OR³, —CH═CH—C(O)—N(R³)(R^{3a}), —N(R³)—C(O)—CF³, —N(R³)—C₂-C₆alkyl-N(R³)(R^{3a}), —C₀-C₆alkyl-N(R³)(R^{3a}), —N(R³)—C(O)—C₁-C₆alkyl-R³, —N(R³)—S(O)₂—C₁-C₆alkyl-R³, —S(O)₂—N(R³)R^{3a}, —O—C₂-C₆alkyl-N(R³)(R^{3a}), —S—R³, —S(O)—C₁-C₆alkyl-R³, —S(O)₂—C₁-C₆alkyl-R³, C₃-C₆cycloalkyl, heterocyclyl, C₄-C₇heterocyclyl-R³, —O—C₂-C₄alkyl-heterocyclyl, —O-heterocyclyl-C(O)—OR³, —O—C₀-C₄alkyl-aryl, —O—C₀-C₄alkyl-heteroaryl, —O—C(O)—NR³—C₀-C₄alkyl-aryl, —O—C(O)—NR³—C₀-C₄alkyl-heteroaryl, —O—C₀-C₄alkyl-heterocyclylaryl, —O—C₀-C₄alkyl-heterocyclyl-heteroaryl, —N(R³)—C₂-C₄alkyl-heterocyclyl, —N(R³)C(O)N(R³)—C₀-C₄alkyl-heterocyclyl-R³, —C₀-C₄alkyl-OC(O)—R³, —C₀-C₄alkyl-N(R³)C(O)—O—R³, —C₀-C₄alkyl-heterocyclyl-C(O)—O—R³, —N(R³)—C₂-C₄alkyl-heterocyclyl, F, Cl, Br, I, NO₂, —CF₃, —SO₃H, —CN, —C₁-C₆ alkylaryl, aryl, heteroaryl, —C₁-C₆ alkylheteroaryl, wherein each alkyl, alkenyl, alkynyl, cycloalkyl, heterocyclyl, aryl and heteroaryl moeity of the aforementioned R⁴ is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁-C₆alkylaryl, aryl, alkylheteroaryl and heteroaryl.

In another preferred embodiment, embodiment KK, of the FTLD targeted agents according to the present disclosure, R^{3a} is independently selected from the group consisting of —H, alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁— C₆alkylaryl, aryl, alkylheteroaryl and heteroaryl, covalent bond, wherein each alkyl, alkenyl, alkynyl, heteroalkyl, cycloalkyl, heterocyclyl, aryl and heteroaryl moiety is optionally substituted with from one to three substituents independently selected from the group consisting of alkyl, heterocyclyl, C₂-C₆alkenyl, C₂-C₃alkynyl, C₂-C₄alkyl-OR¹, heteroalkyl, heteroaryl, C₀-C₆alkylheteroaryl, C(O)CF₃, —C(O)—NH₂, —C₃-C₆cycloalkyl, -alkyl-C₃-C₆cycloalkyl, —C₁— C₆alkylaryl, aryl, alkylheteroaryl and heteroaryl.

In another preferred embodiment, embodiment LL, of the FTLD targeted agents according to the present disclosure, Q is selected from the group consisting of

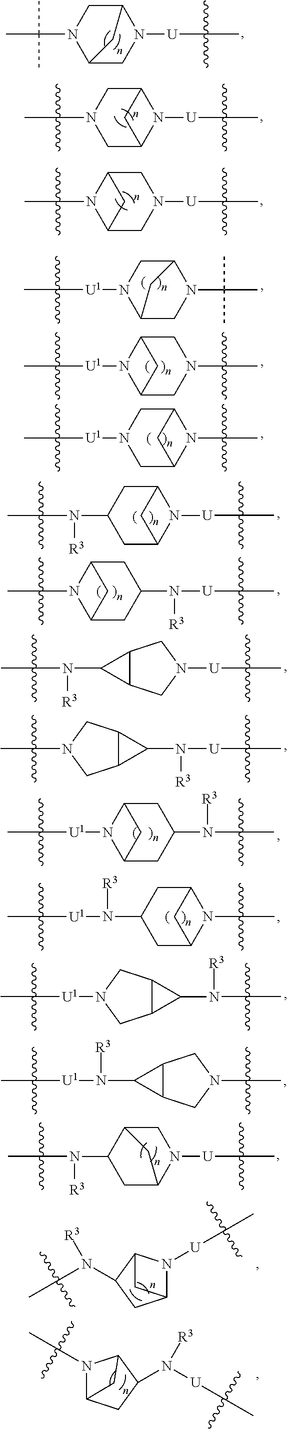

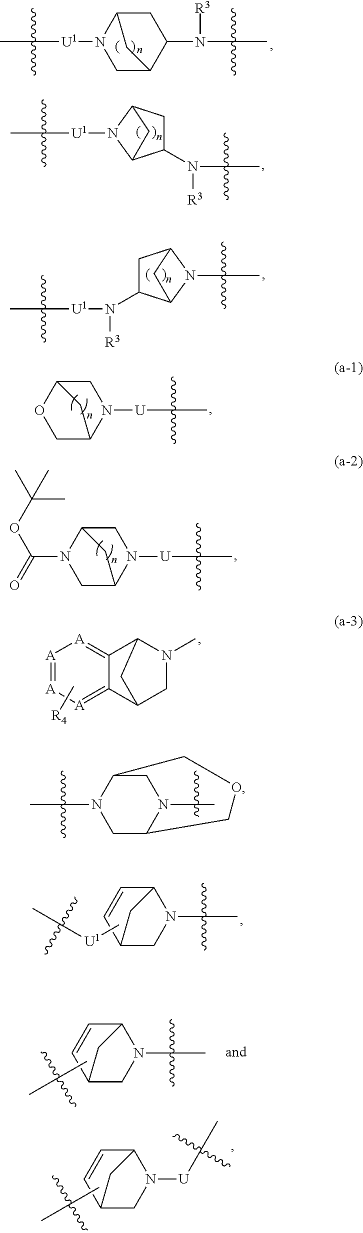

or an optionally substituted (R,R) or (S,S) enantiomer or a mixture of enantiomers, preferably an (R,R) enantiomer, more preferably an (S,S) enantiomer thereof, each of which is optionally substituted with a substituent selected from the group consisting of halo, alkyl and aryl.

In another preferred embodiment, embodiment MM, of the FTLD targeted agents according to the present disclosure,

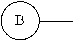

is selected from the group consisting of

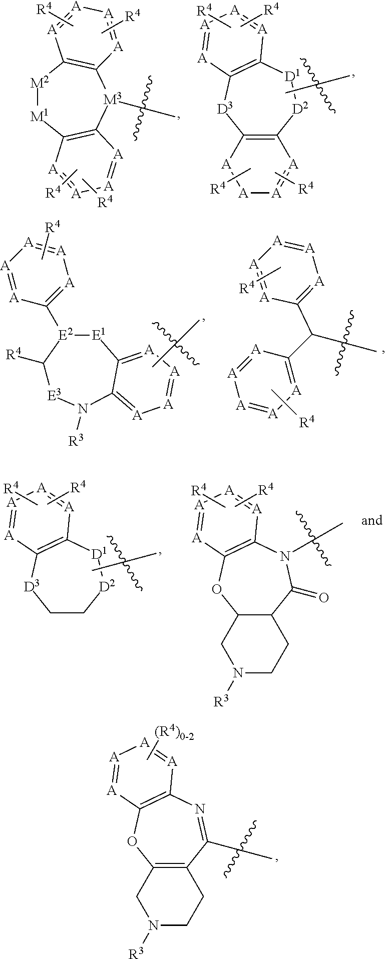

wherein

-M¹-M²- is —CH═CH— or —CH₂—CH₂—;

A is selected from the group consisting of N, C(R⁴) and CH;

Z is —NHOH;

L is covalent bond;

J is selected from the group consisting of —C₁-C₈alkyl-, —C₀-C₆alkyl-aryl-C₀-C₆alkyl-, —C₀-C₆alkyl-aryl-C₂-C₆alkenyl-, —C₀-C₆alkyl-heteroaryl-C₀-C₆alkyl- and —CH═; and

Q is selected from the group consisting of covalent bond, ═N—O—, —C₀-C₆alkyl-N(R³)—C₀-C₃alkyl-, —C₀-C₆alkyl-N(R³)—C(O)—C₀-C₃alkyl- and —C₀-C₆alkyl-C(O)—C₀-C₃alkyl-.

In preferred embodiment of embodiment MM, embodiment MM-1,

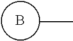

is further selected from the group consisting of

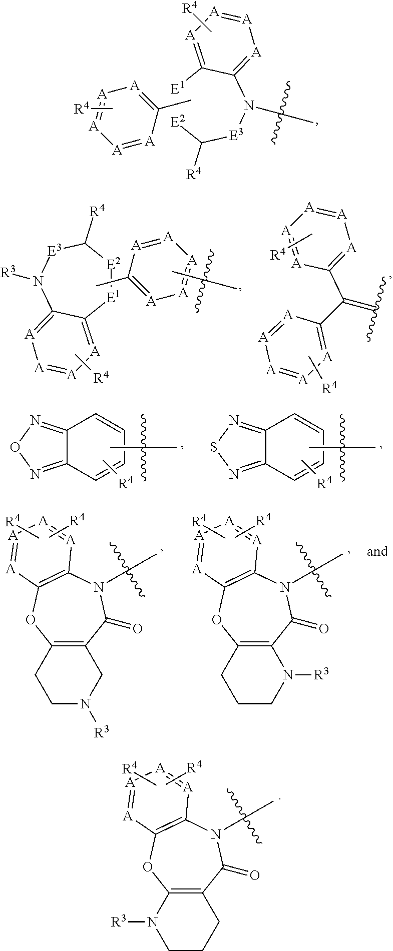

In another preferred embodiment, embodiment NN, of the FTLD targeted agents according to the present disclosure,

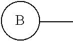

is selected from the group consisting of

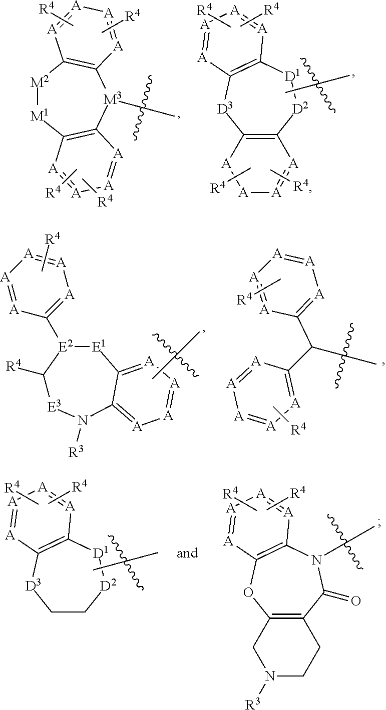

and

Q is —C₀-C₆alkyl-.

In another preferred embodiment, embodiment OO, of the FTLD targeted agents according to the present disclosure,

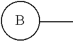

is optionally substituted

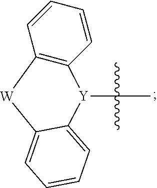

W is —CH═CH— or —CH₂—CH₂—;

Y is selected from the group consisting of N, C(R⁴) and CH;

Z is —NHOH;

L is covalent bond;

J is selected from the group consisting of —C₁-C₈alkyl-, —C₀-C₆alkyl-aryl-C₀-C₆alkyl-, —C₀-C₆alkyl-aryl-C₂-C₆alkenyl-, —C₀-C₆alkyl-heteroaryl-C₀-C₆alkyl- and —CH═; and

Q is selected from the group consisting of covalent bond, ═N—O—, —C₀-C₆alkyl-N(R³)—C₀-C₃alkyl-, —C₀-C₆alkyl-N(R³)—C(O)—C₀-C₃alkyl- and —C₀-C₆alkyl-C(O)—C₀-C₃alkyl-.

In another preferred embodiment, embodiment PP, of the FTLD targeted agents of the present disclosure,

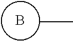

is selected from the group consisting of

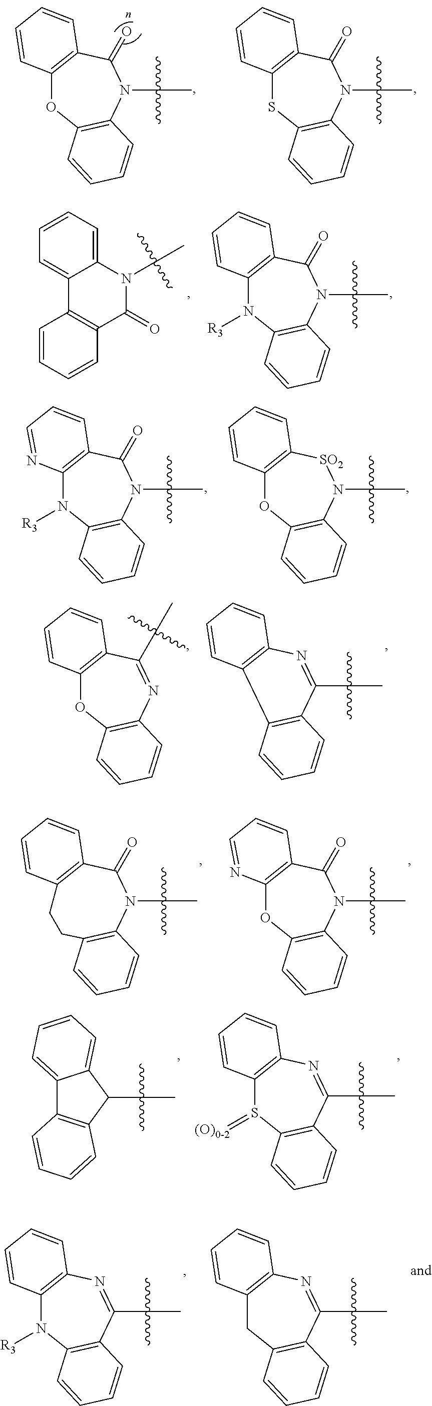

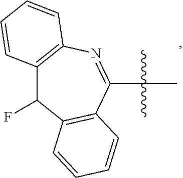 [SMILES: *C1=Nc2ccccc2C(F)c2ccccc21]

each of which is optionally substituted on a phenyl ring with one or two R⁴;

Z is —NR¹OR² or H;

R¹ and R² are —H;

L is covalent bond or —N(OH)—;

J is —C₁-C₈alkyl-, —C₀-C₆alkyl-aryl-C₀-C₆alkyl-, —C₀-C₆alkyl-heteroaryl-C₀-C₆alkyl-, —C₀-C₃alkyl-C₂-C₆alkenyl-C₀-C₃alkyl-, —C₀-C₆alkyl-aryl-C₂-C₆alkenyl- and —C₂-C₆alkenyl-aryl-C₀-C₆alkyl-;

Q is selected from the group consisting of covalent bond, —C₁-C₃alkyl-(C≡C)—C₀-C₃alkyl, —C₀-C₆alkyl-, —C₁-C₃alkyl-(CH═CH)—C₀-C₃alkyl-, —C₂-C₆alkyl-O—C₀-C₃alkyl-, —C₂-C₆alkyl-C(O)—C₀-C₃alkyl- and —C₂-C₆alkyl-heterocyclyl-C₀-C₃alkyl-; or

Q is selected from the group consisting of a covalent bond, —C₁-C₃alkyl-(C≡C)—C₀-C₃alkyl, —C₀-C₆alkyl-, —C₁-C₃alkyl-(CH═CH)—C₀-C₃alkyl-, —C₀-C₆alkyl-O—C₀-C₃alkyl-, —C₀-C₆alkyl-C(O)—C₀-C₃alkyl- and —C₀-C₆alkyl-heterocyclyl-C₀-C₃alkyl- when

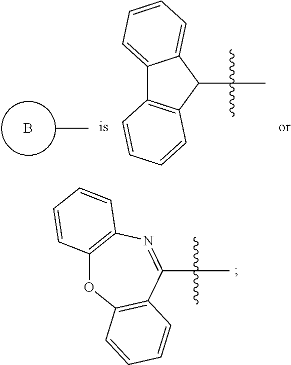

and

R³ is H or cycloalkyl.

In another preferred embodiment, embodiment QQ, of the FTLD targeted agents according to the present disclosure,

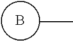

is selected from the group consisting of (aryl)₂-CH—C₀-C₆alkyl-, (aryl)₂-C₁-C₆alkyl- and (heteroaryl)₂-C₁-C₆alkyl-, wherein each aryl, alkyl and heteroaryl moiety is optionally substituted;

Z is NHOH;

Q is selected from the group consisting of —C₀-C₆alkyl-heteroaryl-C₀-C₆alkyl-, ═N—O—, —C₀-C₆alkyl-heterocyclyl-C₀-C₃alkyl and —C₀-C₆alkyl-O—C₀-C₃alkyl;

J is —C₀-C₆alkyl-heteroaryl-C₀-C₆alkyl; and

L is a covalent bond.

In another preferred embodiment, embodiment RR, of the FTLD targeted agents according to the present disclosure,

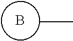

is selected from the group consisting of aryl and (aryl)₂-alkyl, each of which is optionally substituted and H;

Q is selected from the group consisting of —C₀-C₆alkyl-bridged heterocyclyl-C₀-C₃alkyl- and

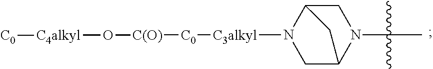

J is —C₀-C₆alkyl-heteroaryl-C₀-C₆alkyl;

L is a covalent bond; and

Z is NHOH.

In another preferred embodiment, embodiment SS, of the FTLD targeted agents according to the present disclosure,

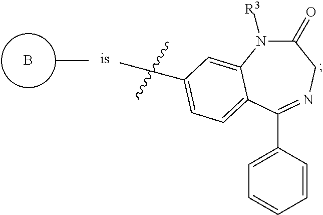

Z is —NHOH;

R³ is H or alkyl;

L is covalent bond;

J is —C₁-C₈alkyl- or —C₀-C₃alkyl-C₁-C₈alkenyl-C₀-C₃alkyl-; and

Q is covalent bond.

In another preferred embodiment, embodiment TT, of the FTLD targeted agents according to the present disclosure,

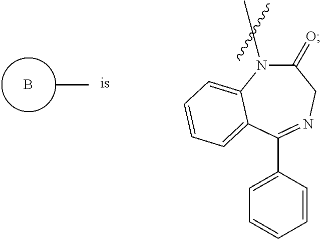

Z is —NHOH;

L is a covalent bond;

J is —C₁-C₈alkyl- or —C₀-C₆alkyl-aryl-C₂-C₆alkenyl-; and

Q is a covalent bond.

In another preferred embodiment, embodiment UU, of the FTLD targeted agents according to the present disclosure, the compound is selected from one of the following structures:

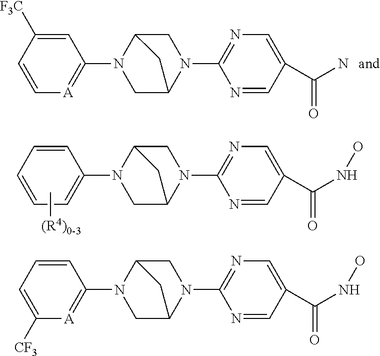

wherein R⁴ is as defined for embodiment (A), and A is selected from the group consisting of N and —CH═.

In another preferred embodiment, embodiment VV, of the FTLD targeted agents according to the present disclosure, the compounds are represented by the Formula II:

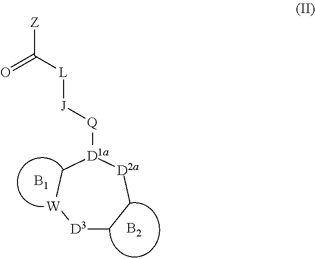

and N-oxides, hydrates, solvates, pharmaceutically acceptable salts, prodrugs, polymorphs and complexes thereof, and racemic and scalemic mixtures, diastereomers and enantiomers thereof, wherein

Z is selected from the group consisting of —N(R¹)OR² and H;

L is selected from the group consisting of a covalent bond and —N(OR²)—;

wherein, when L is —N(OR²)—, then Z is H; and

wherein, when Z is H, then L is —N(OR²)—;

R¹ and R² are independently selected from the group consisting of —H and C₁-C₆alkyl;

W is nitrogen or carbon;

D^{1a}-D^{2a} is selected from the group consisting of

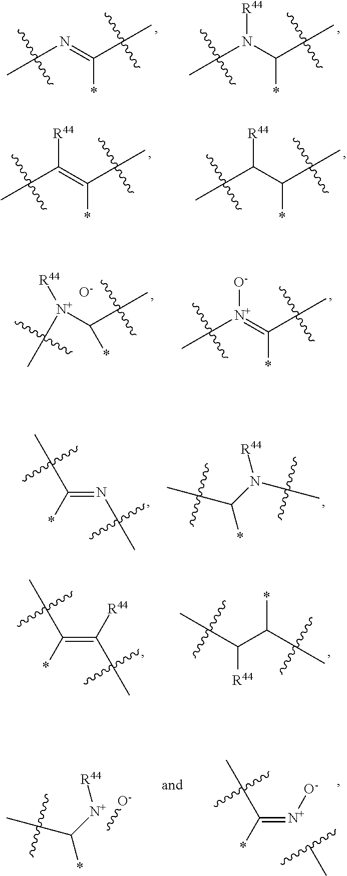

wherein, * represents the point of attachment to Q;

D³ is independently selected from the group consisting of —C(R⁵⁵)(R⁶⁶)—, —C(R⁵⁵)(OH)—, —C(O)—, —O—, —N(R⁷⁷)— and —S(O)₀₋₂—;

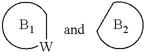

are independently selected from the group consisting of phenyl, heteroaryl and heterocyclyl, wherein each phenyl, heteroaryl and heterocyclyl is optionally substituted with one to three substituents independently selected from the group consisting of halo, —CF₃, —OCF₃, —NO₂, —CN, —C₁-C₆alkyl, —C₁-C₆alkoxyl, —O—C₂-C₆alkyl-O—R⁵³, —O—R⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-C₃-C₇cycloalkyl, —C₀-C₆alkyl-heterocyclyl, —C₀-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³;

R⁴⁴ is independently selected from the group consisting of —H, —C₁-C₆alkyl, —C₀-C₆alkyl-C₃-C₇cycloalkyl and —C₀-C₄alkyl-heterocyclyl;

R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;

or

R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;

R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;

R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl;

R⁵⁵ and R⁶⁶ are independently selected from the group consisting of —H, —C₁-C₆alkyl, —C₁-C₆alkoxyl, —C₀-C₄alkyl-C₃-C₇cycloalkyl and —C₀-C₄alkyl-heterocyclyl;

or

R⁵⁵ and R⁶⁶, together with the atom to which they are attached, optionally form a 3-7 membered cycloalkyl or heterocyclic ring, wherein each cycloalkyl and heterocyclyl is optionally substituted with one to three substituents independently selected from the group consisting of halo, —OH, amino, —CN or —C₁-C₄alkyl;

R⁷⁷ is independently selected from the group consisting of —H, —C₁-C₆alkyl, —C₁-C₆heteroalkyl, —C₃-C₇cycloalkyl, —C(O)—R⁵³, —C(O)O—R⁵³, -cycloalkyl, —C₁-C₄alkyl-cycloalkyl, phenyl, —C₁-C₄alkyl-phenyl, -heterocyclyl, —C₁-C₄alkyl-heterocyclyl and —C₂-C₆alkyl-NR⁸⁸R⁹⁹ wherein each alkyl and heteroalkyl is optionally substituted with one or three substituents independently selected from the group consisting of F, —OH and oxo, wherein each phenyl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;

or R⁷⁷ together with the N to which it is attached may form a ring with

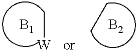

wherein the ring is a 5-7 membered heterocyclic ring, and

R⁸⁸ and R⁹⁹ are independently selected from the group consisting of —H, —C₁-C₆alkyl, —C₂-C₆alkyl-O—C₁-C₆alkyl and —C₀-C₄alkyl-C₃-C₇cycloalkyl, wherein each cycloalkyl and alkyl is optionally substituted with one to three substituents independently selected from the group consisting of halo, —OH, amino, —CN or —C₁-C₆alkyl-aryl;

or

R⁸⁸ and R⁹⁹, together with the N atom to which they are attached, optionally form a 3-10 membered heterocyclic ring, wherein an heterocyclyl is optionally substituted with one to three substituents independently selected from the group consisting of halo, —OH, amino or —CN.

In a preferred embodiment of embodiment VV, embodiment VV-1, of the FTLD targeted agents of the present disclosure,

J-Q is selected from the group consisting of —C₁-C₉alkyl, —C₁-C₉heteroalkyl, phenyl, aryl, heteroaryl, —C₁-C₄alkyl-phenyl, —C₁-C₄alkyl-aryl, —C₁-C₄alkyl-heteroaryl, —NR³³aryl, —NR³³—C₁-C₄alkyl-aryl, —NR³³heteroaryl and NR³³—C₁-C₄alkyl-heteroaryl, wherein each alkyl and heteroalkyl is optionally substituted with one or three substituents independently selected from the group consisting of F, —OH and oxo, wherein each phenyl, aryl and heteroaryl is optionally substituted with one or two substituents independently selected from the group consisting of halo, —OH, —OR⁵³, —C₁-C₄alkyl, —C₁-C₄alkoxyl, —O—C₂-C₄alkyl-O—C₁-C₆alkyl, —CN, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂, wherein R³³ is independently selected from the group consisting of —H, —C₁-C₆alkyl, —C₀-C₆alkyl-C₃-C₇cycloalkyl and —C₀-C₄alkyl-phenyl, wherein each phenyl and cycloalkyl is optionally substituted with one or three substituents independently selected from the group consisting of halo, —OH, —NO₂, —CF₃, —OCF₃, amino, —N(C₁-C₆alkyl)₂, —C₁-C₆alkyl-S(O)₀₋₂R⁵³, —C₁-C₄alkoxyl-CN, —O—C₂alkyl-O—CH₃, —NR⁵⁰R⁵¹, —C₁-C₆alkyl-NR⁵⁰R⁵¹ or —C₁-C₄alkyl.

In a preferred embodiment of embodiment VV, embodiment VV-2, of the FTLD targeted agents of the present disclosure, the moiety

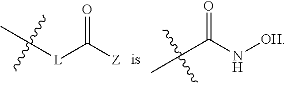

In a preferred embodiment of embodiment VV, embodiment VV-3, of the FTLD targeted agents of the present disclosure,

J-Q is selected from the group consisting of 5- or 6-membered heteroaryl.

In a preferred embodiment of embodiment VV, embodiment VV-4, of the FTLD targeted agents of the present disclosure, the compounds are represented by the Formula (III):

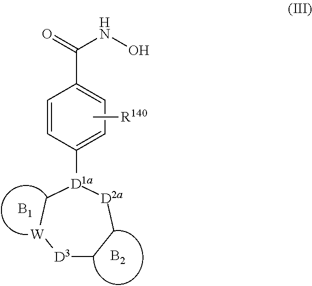

wherein R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂.

In a preferred embodiment of embodiment VV-4, embodiment VV-5, of the compounds of the present disclosure,

D^{1a}-D^{2a} is selected from the group consisting of

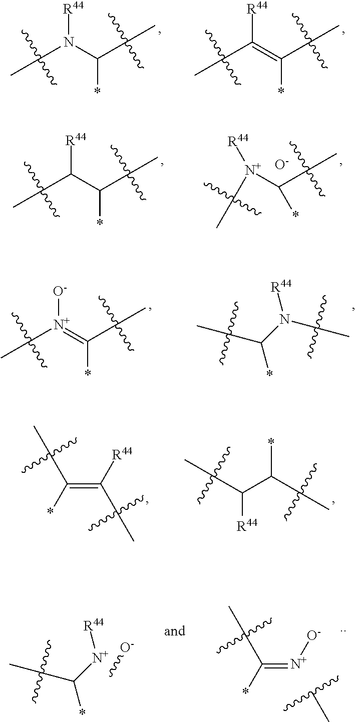

In a preferred embodiment of embodiment VV-4, embodiment VV-6, of the FTLD targeted agents of the present disclosure,

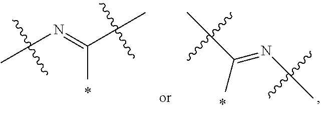

In a preferred embodiment of embodiment VV-4, embodiment VV-7, of the FTLD targeted agents of the present disclosure,

D^{1a}-D^{2a} is

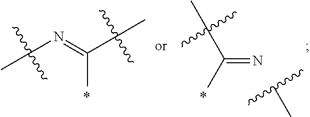

and

D³ is selected from the group consisting of —C(R⁵⁵)(R⁶⁶)—, —C(R⁵⁵)(OH)—, —C(O)—, —O—, —N(R)— and —S(O)₀₋₂.

In a preferred embodiment of embodiment VV-4, embodiment VV-8, of the FTLD targeted agents of the present disclosure,

D^{1a}-D^{2a} is

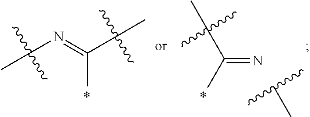

and

D³ is —N(R⁷⁷)—.

In a preferred embodiment of embodiment VV-4, embodiment VV-9, of the FTLD targeted agents of the present disclosure,

D^{1a}-D^{2a} is

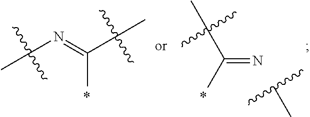

D³ is —O—.

In a preferred embodiment of embodiment VV-4, embodiment VV-10, of the FTLD targeted agents of the present disclosure,

D^{1a}-D^{2a} is

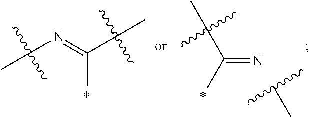

D³ is —O—; and

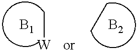

are independently selected from the group consisting of phenyl, pyridyl, pyrimidyl, thienyl, pyrazolyl, thiazyl and oxazyl.

In a preferred embodiment of embodiment VV-4, embodiment VV-11, of the FTLD targeted agents of the present disclosure,

D^{1a}-D^{2a} is

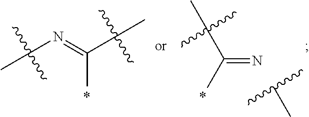

D³ is —O—; and

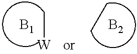

are independently selected from the group consisting of phenyl, pyridyl, pyrimidyl, thienyl, pyrazolyl, thiazyl and oxazyl, wherein at least one of and

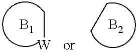

is phenyl, wherein the phenyl, pyridyl, pyrimidyl, thienyl, pyrazolyl, thiazyl and oxazyl are independently optionally substituted.

In a preferred embodiment of embodiment VV-4, embodiment VV-12, of the FTLD targeted agents of the present disclosure, D^{1a}-D^{2a} is

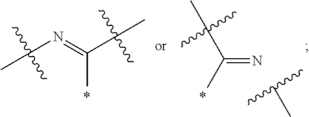

D³ is —N(R⁷⁷)—; and

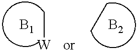

are independently selected from the group consisting of phenyl, pyridyl, pyrimidyl and thienyl.

In a preferred embodiment of embodiment VV-4, embodiment VV-13, of the FTLD targeted agents of the present disclosure,

D^{1a}-D^{2a} is

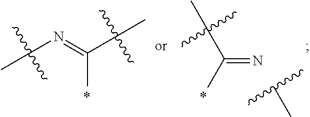

D³ is —N(R⁷⁷)—; and

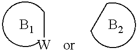

are independently selected from the group consisting of phenyl, pyridyl, pyrimidyl and thienyl, wherein at least one of

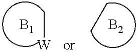

is phenyl, wherein said phenyl, pyridyl, pyrimidyl and thienyl are independently optionally substituted.

In a preferred embodiment of embodiment VV, embodiment VV-14, of the FTLD targeted agents of the present disclosure, the compounds are represented by the Formula (IV):

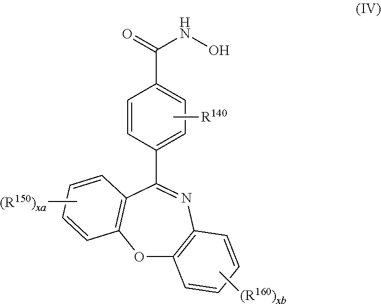

wherein R¹⁴⁰, is as defined in Formula III;

xa and xb denote numbers that are each independently selected from 0, 1 and 2; and

R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂.

In a preferred embodiment of embodiment VV, embodiment VV-15, of the FTLD targeted agents of the present disclosure, the compounds are represented by the Formula (V):

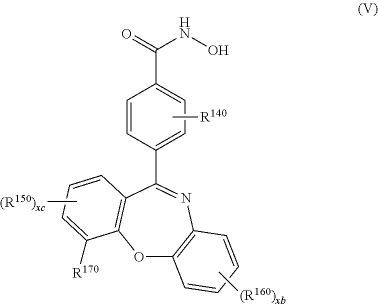

wherein R¹⁴⁰ is as defined in Formula III, and xb, R¹⁵⁰ and R¹⁶⁰ are as defined in Formula IV;

xc is 0 or 1; and

R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂, wherein R⁵⁰, R⁵¹, R⁵², and R⁵³ are as defined in Formula II.

In a preferred embodiment of embodiment VV, embodiment VV-16, of the FTLD targeted agents of the present disclosure, the compounds represented by the Formula (VI):

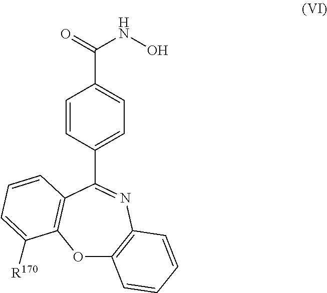

wherein R¹⁷⁰ is as defined in Formula V.

In a preferred embodiment of embodiment VV, embodiment VV-17, of the FTLD targeted agents of the present disclosure, the compounds are represented by the Formula (VII):

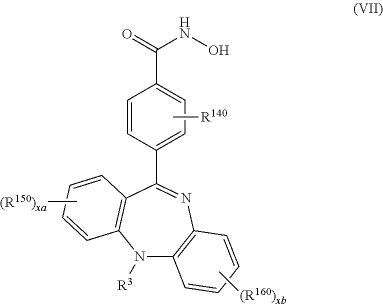

wherein R¹⁴⁰ is as defined in Formula III, xa, xb, R¹⁵⁰ and R¹⁶⁰ are as defined in Formula IV; and R³ is as defined in Formula I.

In a preferred embodiment of embodiment VV, embodiment VV-18, of the FTLD targeted agents of the present disclosure, R³ is R¹⁸⁰, wherein

R¹⁸⁰ is selected from the group consisting of H, —C₁-C₆alkyl, —C₁-C₆alkenyl, —C₁-C₆alkynyl, —C₂-C₆alkoxyl, —C₂-C₆alkyl-O—R⁵³, —OR⁵³, —C₂-C₆alkyl-S(O)₀₋₂—R⁵³, —C₂-C₆alkyl-C(O)—R⁵³, —C₂-C₆alkyl-C(O)NR⁵⁰R⁵¹, —C₂-C₆alkyl-NR⁵²C(O)—R⁵³, —C₂-C₆alkyl-SO)₀₋₂NR⁵⁰R⁵¹, —C₂-C₆alkyl-NR⁵²S(O)₂—R⁵³, —C₂-C₆alkyl-OC(O)NR⁵⁰R⁵¹, —C₂-C₆alkyl-NR⁵²C(O)O—R⁵³, —C₂-C₆alkyl-NR⁵²C(O)NR⁵⁰R⁵¹, —C₂-C₆alkyl-C(O)O—R⁵³, —C₂-C₆alkyl-OC(O)—R⁵³, —C₀-C₆alkyl-heterocyclyl-R⁵³, —C₀-C₆alkyl-heterocyclyl-O—R⁵³, —C₀-C₆alkyl-heterocyclyl-S(O)₀₋₂—R⁵³, —C₀-C₆alkyl-heterocyclyl-C(O)—R⁵³, —C₀-C₆alkyl-heterocyclyl-C(O)NR⁵⁰R⁵¹, —C₀-C₆alkyl-heterocyclyl-NR⁵²C(O)—R⁵³, —C₀-C₆alkyl-heterocyclyl-S(O)₂NR⁵⁰R⁵¹, —C₀-C₆alkyl-heterocyclyl-NR⁵²S(O)₂—R⁵³, —C₀-C₆alkyl-heterocyclyl-OC(O)NR⁵⁰R⁵¹, —C₀-C₆alkyl-heterocyclyl-NR⁵²C(O)O—R⁵³, —C₀-C₆alkyl-heterocyclyl-NR⁵²C(O)NR⁵⁰R⁵¹, —C₀-C₆alkyl-heterocyclyl-C(O)O—R⁵³, —C₀-C₆alkyl-heterocyclyl-OC(O)—R⁵³, —C₀-C₆alkyl-cycloalkyl-R⁵³, —C₀-C₆alkyl-cycloalkyl-O—R⁵³, —C₀-C₆alkyl-cycloalkyl-S(O)₀₋₂—R⁵³, —C₀-C₆alkyl-cycloalkyl-C(O)—R⁵³, —C₀-C₆alkyl-cycloalkyl-C(O)NR⁵⁰R⁵¹, —C₀-C₆alkyl-cycloalkyl-NR⁵²C(O)—R⁵³, —C₀-C₆alkyl-cycloalkyl-S(O)₂NR⁵⁰R⁵¹, —C₀-C₆alkyl-cycloalkyl-NR⁵²S(O)₂—R⁵³, —C₀-C₆alkyl-cycloalkyl-OC(O)NR⁵⁰R⁵¹, —C₀-C₆alkyl-cycloalkyl-NR⁵²C(O)O—R⁵³, —C₀-C₆alkyl-cycloalkyl-NR⁵²C(O)NR⁵⁰R⁵¹, —C₀-C₆alkyl-cycloalkyl-C(O)O—R⁵³, —C₀-C₆alkyl-cycloalkyl-OC(O)—R⁵³, —C₀-C₆alkyl-heteroaryl-R⁵³, —C₀-C₆alkyl-heteroaryl-O—R⁵³, —C₀-C₆alkyl-heteroaryl-S(O)₀₋₂—R⁵³, —C₀-C₆alkyl-heteroaryl-C(O)—R⁵³, —C₀-C₆alkyl-heteroaryl-C(O)NR⁵⁰R⁵¹, —C₀-C₆alkyl-heteroaryl-NR⁵²C(O)—R⁵³, —C₀-C₆alkyl-heteroaryl-S(O)₂NR⁵⁰R⁵¹, —C₀-C₆alkyl-heteroaryl-NR⁵²S(O)₂—R⁵³, —C₀-C₆alkyl-heteroaryl-OC(O)NR⁵⁰R⁵¹, —C₀-C₆alkyl-heteroaryl-NR⁵²C(O)O—R⁵³, —C₀-C₆alkyl-heteroaryl-NR⁵²C(O)NR⁵⁰R⁵¹, —C₀-C₆alkyl-heteroaryl-C(O)O—R⁵³, —C₀-C₆alkyl-heteroaryl-OC(O)—R⁵³, —C₀-C₆alkyl-aryl-R⁵³, —C₀-C₆alkyl-aryl-O—R⁵³, —C₀-C₆alkyl-aryl-S(O)₀₋₂—R⁵³, —C₀-C₆alkyl-aryl-C(O)—R⁵³, —C₀-C₆alkyl-aryl-C(O)NR⁵⁰R⁵¹, —C₀-C₆alkyl-aryl-NR⁵²C(O)—R⁵³, —C₀-C₆alkyl-aryl-S(O)₂NR⁵⁰R⁵¹, —C₀-C₆alkyl-aryl-NR⁵²S(O)₂—R⁵³, —C₀-C₆alkyl-aryl-OC(O)NR⁵⁰R⁵¹, —C₀-C₆alkyl-aryl-NR⁵²C(O)O—R⁵³, —C₀-C₆alkyl-aryl-NR⁵²C(O)NR⁵⁰R⁵¹, —C₀-C₆alkyl-aryl-C(O)O—R⁵³, —C₀-C₆alkyl-aryl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl and —C₂-C₆alkyl-NR⁵⁰R⁵¹, wherein each alkyl and heteroalkyl is optionally substituted with one to three substituents independently selected from the group consisting of F, —OH and oxo, wherein each aryl, heteroaryl, cycloalkyl and heterocyclyl is optionally substituted with one or two substituents.

In a preferred embodiment of embodiment VV, embodiment VV-19, the FTLD targeted agent is selected from the group consisting of:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide, and
(Z)—N-hydroxy-4-(5-(2-morpholinoethyl)-5H-dibenzo[b,e][1,4]diazepin-11-yl)benzamide.

In a preferred embodiment, embodiment WW, of the FTLD targeted agents according to the present disclosure, the compounds are represented by the Formula VIII:

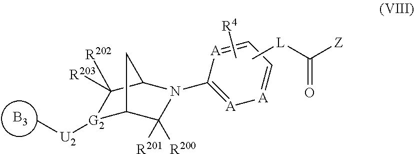

and N-oxides, hydrates, solvates, pharmaceutically acceptable salts, prodrugs, polymorphs and complexes thereof, and racemic and scalemic mixtures, diastereomers and enantiomers thereof, wherein

wherein R⁴ and A are as defined in Formula I;

Z is —N(R¹)OR² or H;

L is a covalent bond or —C₀-C₃alkyl-N(OR²)—;

wherein, when L is C₀-C₃alkyl-N(OR²)—, then Z is H; and

wherein, when Z is H, then L is —C₀-C₃alkyl-N(OR²)—;

G² is carbon or N;

U² is selected from the group consisting of a covalent bond, —C₁-C₈alkyl-, —C(R³⁰⁰)(R⁴⁰⁰)—, —C(O)—C(R³⁰¹)(R⁴⁰¹)—, —C₀-C₂alkyl-C(O)—O—C₀-C₄alkyl-, —C₀-C₂alkyl-C(O)—C₀-C₄alkyl-, —C₀-C₂alkyl-C(O)—NR³—C₀-C₄alkyl-, —C(O)—O—C(R³⁰¹)(R⁴⁰¹)—, —C(O)—C(R³⁰¹)(R⁴⁰¹)— and —C(O)—NR³—C(R³⁰⁰)(R⁴⁰⁰)—,

wherein R³ and R^{3a} are as defined in Formula I;

R³⁰⁰ and R⁴⁰⁰ are independently selected from the group consisting of —H, —F, —C₁-C₆alkyl, aryl, heteroaryl, heterocyclyl and cycloalkyl;

R³⁰¹ and R⁴⁰¹ are independently selected from the group consisting of —H, F, OR¹, —NR³R^{3a}—, —C₁-C₆alkyl, aryl, heteroaryl, heterocyclyl and cycloalkyl;

R²⁰⁰, R²⁰¹, R²⁰² and R²⁰³ are independently selected from the group consisting of —H, —C₁-C₆alkyl, aryl, heteroaryl, heterocyclyl and cycloalkyl; and

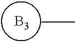

is selected from the group consisting of hydrogen, aryl, heteroaryl, alkyl, heterocyclyl, cycloalkyl, wherein each aryl, heteroaryl, cycloalkyl and heterocyclyl is optionally substituted with one to three substituents independently selected from the group consisting of halo, —CF₃, —OCF₃, —SCF₃, —SF₅, —NO₂, —CN, —C₁-C₆alkyl, —C₁-C₆alkoxyl, —O—C₂-C₆alkyl-O—R¹, —O—R¹, —OCF₂H, —C₀-C₆alkyl-S(O)₀₋₂—R¹, —C₀-C₆alkyl-C(O)—R¹, —C₀-C₆alkyl-C(O)NR³R^{3a}, —C₀-C₆alkyl-NR³C(O)—R², —C₀-C₆alkyl-S(O)₂NR³R^{3a}, —C₀-C₆alkyl-NR³S(O)₂—R², —C₀-C₆alkyl-OC(O)NR³R^{3a}, —C₀-C₆alkyl-NR³C(O)O—R¹, —C₀-C₆alkyl-NR¹C(O)NR³R^{3a}, —C₀-C₆alkyl-C(O)O—R¹, —C₀-C₆alkyl-OC(O)—R¹, —C₀-C₆alkyl-aryl, —C₀-C₆alkyl-heteroaryl, —C₀-C₆alkyl-C₃-C₇cycloalkyl, —C₀-C₆alkyl-heterocyclyl, —C₀-C₆alkyl-NR³R^{3a} and —O—C₂-C₆alkyl-NR³R^{3a}.
In a preferred embodiment of embodiment WW, embodiment WW-1, the moiety

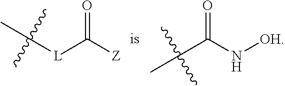

In a preferred embodiment of embodiment WW, embodiment WW-2, the moiety

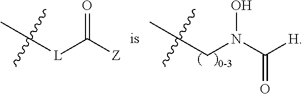

In a preferred embodiment of embodiment WW, embodiment WW-3, the moiety

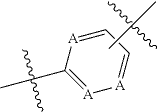

is a radical selected from the group consisting of

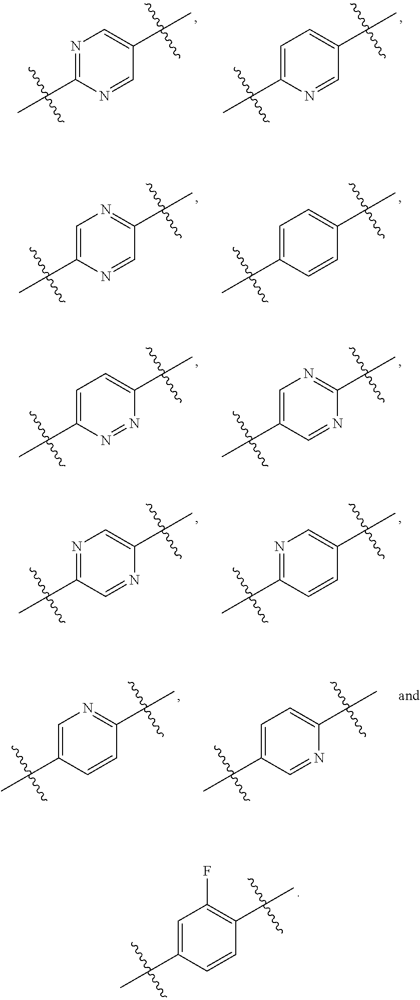

In a preferred embodiment of embodiment WW, embodiment WW-4, the moiety

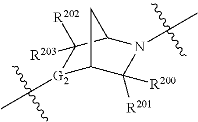

is a radical

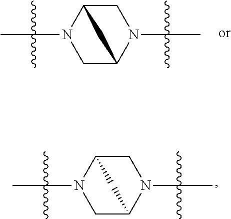 [SMILES: *N1C[C@@H]2C[C@H]1CN2* ; *N1C[C@H]2C[C@@H]1CN2*]

or an enantiomer thereof, a scalemic thereof, or a mixture of enantiomers thereof.

In a preferred embodiment of embodiment WW, embodiment WW-5, U² is a covalent bond.

In a preferred embodiment of embodiment WW, embodiment WW-6, U² is selected from the group consisting of a —C₁-C₄alkyl, —CH(aryl)-, —CH(heteroaryl)-, —C(O)—, —C(O)—CH(aryl)-, —C(O)—CH(heteroaryl)-, —C(O)O—C₁-C₂alkyl-, —C(O)O— and —C(O)NH—.

In a preferred embodiment of embodiment WW, embodiment WW-7, the moiety

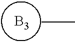

is a radical selected from the group consisting of H, alkyl, aryl, heteroaryl, cycloalkyl and heterocyclyl, wherein each aryl, heteroaryl, cycloalkyl and heterocyclyl is optionally substituted with one to three substituents independently selected from the group consisting of halo, —CF₃, —OCF₃, —SCF₃, —SF₅, —CN, —C₁-C₆alkyl, —O—C₂-C₆alkyl-O—R¹, —O—R¹, —OCF₂H, —C₀-C₆alkyl-S(O)₀₋₂—R¹, —C₀-C₆alkyl-C(O)NR³R^{3a}, —C₀-C₆alkyl-NR³C(O)—R², —C₀-C₆alkyl-S(O)₂NR³R^{3a}, —C₀-C₆alkyl-NR³S(O)₂—R², —C₀-C₆alkyl-OC(O)NR³R^{3a}, —C₀-C₆alkyl-NR³C(O)O—R¹, —C₀-C₆alkyl-NR¹C(O)NR³R^{3a}, —C₀-C₆alkyl-C(O)O—R¹, —C₀-C₆alkyl-OC(O)—R¹, —C₀-C₆alkyl-aryl, —C₀-C₆alkyl-heteroaryl, —C₀-C₆alkyl-C₃-C₇cycloalkyl, —C₀-C₆alkyl-heterocyclyl, —C₀-C₆alkyl-NR³R^{3a} and —O—C₂-C₆alkyl-NR³R^{3a}.

In a preferred embodiment of embodiment WW, embodiment WW-8, the moiety

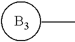

is a radical selected from the group consisting of

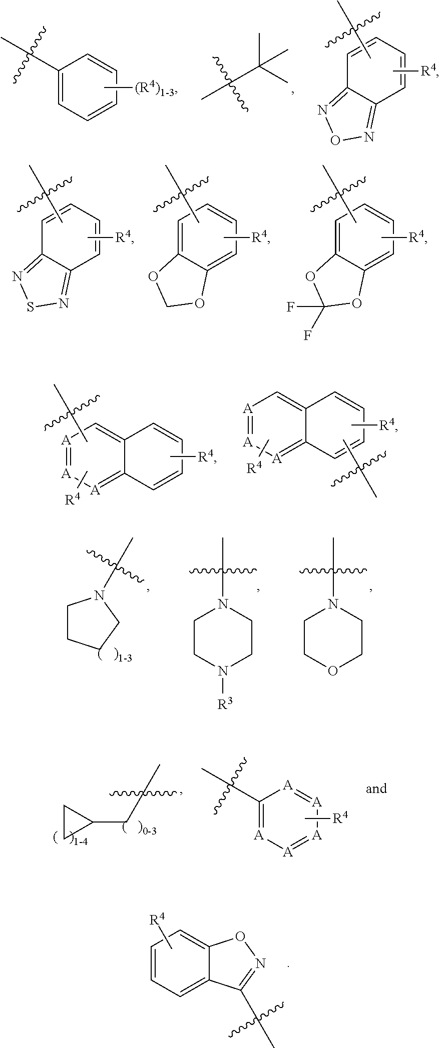

In a preferred embodiment of embodiment WW, embodiment WW-9, the FTLD targeted agents are represented by the Formula (IX):

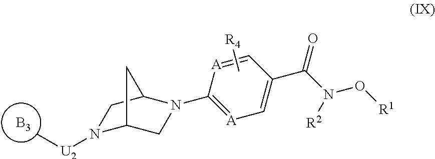

or where possible, a (R,R) or (S,S) enantiomer, scalemic or a mixture of enantiomers thereof,

wherein

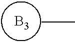

and U₂ are as defined in Formula (VIII); and

A, R¹, R² and R⁴ are as defined in Formula I.

In a preferred embodiment of embodiment WW, embodiment WW-10, the FTLD targeted agents are represented by the Formula (X):

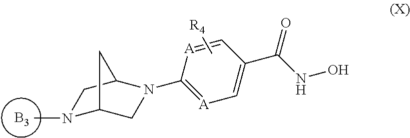

or where possible, a (R,R) or (S,S) enantiomer, scalemic or a mixture of enantiomers thereof,

wherein

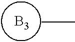

is as defined in Formula (VIII); and

A and R⁴ are as defined in Formula I.

In a preferred embodiment of embodiment WW, embodiment WW-11, the moiety

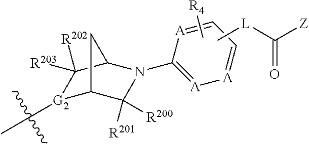

is a radical selected from the group consisting of

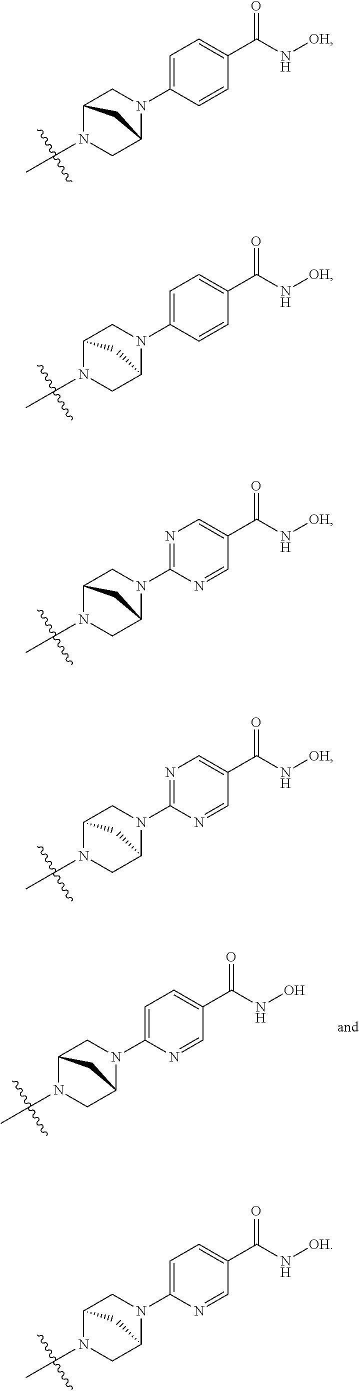 [SMILES: *N1C[C@@H]2C[C@H]1CN2c1ccc(C(=O)NO)cc1 ; *N1C[C@H]2C[C@@H]1CN2c1ccc(C(=O)NO)cc1 ; *N1C[C@@H]2C[C@H]1CN2c1ncc(C(=O)NO)cn1 ; *N1C[C@H]2C[C@@H]1CN2c1ncc(C(=O)NO)cn1 ; *N1C[C@@H]2C[C@H]1CN2c1ccc(C(=O)NO)cn1 ; *N1C[C@H]2C[C@@H]1CN2c1ccc(C(=O)NO)cn1]

In a preferred embodiment of embodiment WW, embodiment WW-12, the FTLD targeted agent is selected from the group consisting of:
2-((1S,4S)-5-benzyl-2,5-diazabicyclo[2.2.1]heptan-2-yl)-N-hydroxypyrimidine-5-carboxamide,
N-hydroxy-2-((1S,4S)-5-p-tolyl-2,5-diazabicyclo[2.2.1]heptan-2-yl)pyrimidine-5-carboxamide,
2-((1S,4S)-5-benzhydryl-2,5-diazabicyclo[2.2.1]heptan-2-yl)-N-hydroxypyrimidine-5-carboxamide,
2-((1S,4S)-5-(4-chlorophenyl)-2,5-diazabicyclo[2.2.1]heptan-2-yl)-N-hydroxypyrimidine-5-carboxamide,
(1S,4S)-tert-butyl 5-(5-(hydroxycarbamoyl)pyrimidin-2-yl)-2,5-diazabicyclo[2.2.1]heptane-2-carboxylate,
2-((1S,4S)-5-(3-fluorophenyl)-2,5-diazabicyclo[2.2.1]heptan-2-yl)-N-hydroxypyrimidine-5-carboxamide,
2-((1S,4S)-5-(4-fluorophenyl)-2,5-diazabicyclo[2.2.1]heptan-2-yl)-N-hydroxypyrimidine-5-carboxamide,
2-((1S,4S)-2,5-diazabicyclo[2.2.1]heptan-2-yl)-N-hydroxypyrimidine-5-carboxamide,
N-hydroxy-2-((1S,4S)-5-o-tolyl-2,5-diazabicyclo[2.2.1]heptan-2-yl)pyrimidine-5-carboxamide,
N-hydroxy-2-((1S,4S)-5-phenyl-2,5-diazabicyclo[2.2.1]heptan-2-yl)pyrimidine-5-carboxamide,
2-((1S,4S)-5-benzoyl-2,5-diazabicyclo[2.2.1]heptan-2-yl)-N-hydroxypyrimidine-5-carboxamide,
N-hydroxy-2-((1S,4S)-5-(3-(trifluoromethyl)phenyl)-2,5-diazabicyclo[2.2.1]heptan-2-yl)pyrimidine-5-carboxamide,
2-((1S,4S)-5-(2-fluoro-4-(trifluoromethyl)phenyl)-2,5-diazabicyclo[2.2.1]heptan-2-yl)-N-hydroxypyrimidine-5-carboxamide,
N-hydroxy-2-((1S,4S)-5-(2-(trifluoromethyl)phenyl)-2,5-diazabicyclo[2.2.1]heptan-2-yl)pyrimidine-5-carboxamide,
N-hydroxy-2-((1S,4S)-5-(4-(trifluoromethyl)phenyl)-2,5-diazabicyclo[2.2.1]heptan-2-yl)pyrimidine-5-carboxamide,
2-((1S,4S)-5-(benzo[c][1,2,5]oxadiazol-5-yl)-2,5-diazabicyclo[2.2.1]heptan-2-yl)-N-hydroxypyrimidine-5-carboxamide,
2-((1S,4S)-5-(benzo[c][1,2,5]thiadiazol-5-yl)-2,5-diazabicyclo[2.2.1]heptan-2-yl)-N-hydroxypyrimidine-5-carboxamide,
N-hydroxy-2-((1S,4S)-5-(3-(trifluoromethyl)benzoyl)-2,5-diazabicyclo[2.2.1]heptan-2-yl)pyrimidine-5-carboxamide,
2-((1S,4S)-5-(benzo[d][1,3]dioxol-5-yl)-2,5-diazabicyclo[2.2.1]heptan-2-yl)-N-hydroxypyrimidine-5-carboxamide,
2-((1S,4S)-5-(cyclohexanecarbonyl)-2,5-diazabicyclo[2.2.1]heptan-2-yl)-N-hydroxypyrimidine-5-carboxamide,
2-((1S,4S)-5-(2,2-diphenylacetyl)-2,5-diazabicyclo[2.2.1]heptan-2-yl)-N-hydroxypyrimidine-5-carboxamide,
N-hydroxy-4-((1S,4S)-5-p-tolyl-2,5-diazabicyclo[2.2.1]heptan-2-yl)benzamide,
(1S,4S)-benzyl 5-(5-(hydroxycarbamoyl)pyrimidin-2-yl)-2,5-diazabicyclo[2.2.1]heptane-2-carboxylate,
(1S,4S)-isobutyl 5-(5-(hydroxycarbamoyl)pyrimidin-2-yl)-2,5-diazabicyclo[2.2.1]heptane-2-carboxylate,
N-hydroxy-2-((1S,4S)-5-(3-(trifluoromethoxy)phenyl)-2,5-diazabicyclo[2.2.1]heptan-2-yl)pyrimidine-5-carboxamide,
2-((1S,4S)-5-(2,2-difluorobenzo[d][1,3]dioxol-5-yl)-2,5-diazabicyclo[2.2.1]heptan-2-yl)-N-hydroxypyrimidine-5-carboxamide,
N-hydroxy-2-((1S,4S)-5-(3-(trifluoromethylthio)phenyl)-2,5-diazabicyclo[2.2.1]heptan-2-yl)pyrimidine-5-carboxamide,
N-hydroxy-2-((1S,4S)-5-(4-(trifluoromethyl)pyridin-2-yl)-2,5-diazabicyclo[2.2.1]heptan-2-yl)pyrimidine-5-carboxamide,
N-hydroxy-2-((1S,4S)-5-(2-(trifluoromethyl)quinolin-4-yl)-2,5-diazabicyclo[2.2.1]heptan-2-yl)pyrimidine-5-carboxamide,
2-((1S,4S)-5-(3-(difluoromethoxy)phenyl)-2,5-diazabicyclo[2.2.1]heptan-2-yl)-N-hydroxypyrimidine-5-carboxamide,
N-hydroxy-2-((1S,4S)-5-(6-(trifluoromethyl)pyridin-2-yl)-2,5-diazabicyclo[2.2.1]heptan-2-yl)pyrimidine-5-carboxamide,
(1S,4S)-cyclopentyl 5-(5-(hydroxycarbamoyl)pyrimidin-2-yl)-2,5-diazabicyclo[2.2.1]heptane-2-carboxylate,
2-((1S,4S)-5-(benzo[c][1,2,5]oxadiazol-4-yl)-2,5-diazabicyclo[2.2.1]heptan-2-yl)-N-hydroxypyrimidine-5-carboxamide,
N-hydroxy-2-((1S,4S)-5-(5-(trifluoromethyl)pyridin-3-yl)-2,5-diazabicyclo[2.2.1]heptan-2-yl)pyrimidine-5-carboxamide,
N-hydroxy-2-((1R,4R)-5-p-tolyl-2,5-diazabicyclo[2.2.1]heptan-2-yl)pyrimidine-5-carboxamide,
(1S,4S)-isopropyl 5-(5-(hydroxycarbamoyl)pyrimidin-2-yl)-2,5-diazabicyclo[2.2.1]heptane-2-carboxylate,
(1S,4S)-pyridin-3-ylmethyl 5-(5-(hydroxycarbamoyl)pyrimidin-2-yl)-2,5-diazabicyclo[2.2.1]heptane-2-carboxylate,
(1S,4S)-cyclopropylmethyl 5-(5-(hydroxycarbamoyl)pyrimidin-2-yl)-2,5-diazabicyclo[2.2.1]heptane-2-carboxylate,
(1S,4S)-tetrahydro-2H-pyran-4-yl 5-(5-(hydroxycarbamoyl)pyrimidin-2-yl)-2,5-diazabicyclo[2.2.1]heptane-2-carboxylate,
2-((1S,4S)-5-(3,5-bis(trifluoromethyl)phenyl)-2,5-diazabicyclo[2.2.1]heptan-2-yl)-N-hydroxypyrimidine-5-carboxamide,
2-((1S,4S)-5-(benzo[d]isoxazol-3-yl)-2,5-diazabicyclo[2.2.1]heptan-2-yl)-N-hydroxypyrimidine-5-carboxamide,
2-((1S,4S)-5-(3-(dimethylcarbamoyl)phenyl)-2,5-diazabicyclo[2.2.1]heptan-2-yl)-N-hydroxypyrimidine-5-carboxamide,
2-((1S,4S)-5-(3-((dimethylamino)methyl)phenyl)-2,5-diazabicyclo[2.2.1]heptan-2-yl)-N-hydroxypyrimidine-5-carboxamide,
N-hydroxy-2-((1S,4S)-5-(3-methoxyphenyl)-2,5-diazabicyclo[2.2.1]heptan-2-yl)pyrimidine-5-carboxamide,
N-hydroxy-2-((1S,4S)-5-m-tolyl-2,5-diazabicyclo[2.2.1]heptan-2-yl)pyrimidine-5-carboxamide,
N-hydroxy-6-(5-p-tolyl-2,5-diazabicyclo[2.2.1]heptan-2-yl)nicotinamide,
N-hydroxy-5-((1S,4S)-5-(3-(trifluoromethyl)phenyl)-2,5-diazabicyclo[2.2.1]heptan-2-yl)pyrazine-2-carboxamide,
2-fluoro-N-hydroxy-4-((1S,4S)-5-(3-(trifluoromethyl)phenyl)-2,5-diazabicyclo[2.2.1]heptan-2-yl)benzamide,
N-hydroxy-2-((1S,4S)-5-(pyrrolidine-1-carbonyl)-2,5-diazabicyclo[2.2.1]heptan-2-yl)pyrimidine-5-carboxamide,
N-hydroxy-2-((1S,4S)-5-(4-(trifluoromethyl)pyrimidin-2-yl)-2,5-diazabicyclo[2.2.1]heptan-2-yl)pyrimidine-5-carboxamide,
N-hydroxy-6-((1S,4S)-5-(3-(trifluoromethyl)phenyl)-2,5-diazabicyclo[2.2.1]heptan-2-yl)pyridazine-3-carboxamide,
N-hydroxy-2-((1R,4R)-5-(4-(trifluoromethyl)pyridin-2-yl)-2,5-diazabicyclo[2.2.1]heptan-2-yl)pyrimidine-5-carboxamide,
N-hydroxy-2-((1R,4R)-5-m-tolyl-2,5-diazabicyclo[2.2.1]heptan-2-yl)pyrimidine-5-carboxamide,
2-(5-(3-cyanophenyl)-2,5-diazabicyclo[2.2.1]heptan-2-yl)-N-hydroxypyrimidine-5-carboxamide,
N-hydroxy-4-(5-(3-methoxyphenyl)-2,5-diazabicyclo[2.2.1]heptan-2-yl)benzamide,
N-hydroxy-4-(5-m-tolyl-2,5-diazabicyclo[2.2.1]heptan-2-yl)benzamide,
N-hydroxy-4-((1S,4S)-5-(3-(trifluoromethyl)phenyl)-2,5-diazabicyclo[2.2.1]heptan-2-yl)benzamide,
N-hydroxy-4-((1S,4S)-5-(4-(trifluoromethyl)pyridin-2-yl)-2,5-diazabicyclo[2.2.1]heptan-2-yl)benzamide,
4-((1S,4S)-5-(3-cyanophenyl)-2,5-diazabicyclo[2.2.1]heptan-2-yl)-N-hydroxybenzamide,
N-hydroxy-4-((1R,4R)-5-m-tolyl-2,5-diazabicyclo[2.2.1]heptan-2-yl)benzamide,
N-hydroxy-4-((1R,4R)-5-(4-(trifluoromethyl)pyridin-2-yl)-2,5-diazabicyclo[2.2.1]heptan-2-yl)benzamide,
N-hydroxy-4-((1S,4S)-5-(4-(trifluoromethyl)pyrimidin-2-yl)-2,5-diazabicyclo[2.2.1]heptan-2-yl)benzamide,
N-hydroxy-N-methyl-4-((1S,4S)-5-p-tolyl-2,5-diazabicyclo[2.2.1]heptan-2-yl)benzamide and

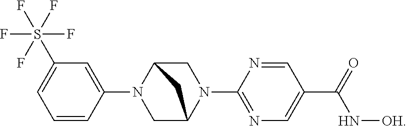 [SMILES: O=C(NO)c1cnc(N2C[C@@H]3C[C@H]2CN3c2cccc(S(F)(F)(F)(F)F)c2)nc1]

In a preferred embodiment of the FTLD targeted agents according to the present disclosure, embodiment XX, the compounds are represented by the Formula (XI):

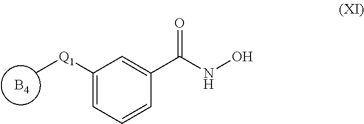

and N-oxides, hydrates, solvates, pharmaceutically acceptable salts, prodrugs, polymorphs and complexes thereof, and racemic and scalemic mixtures, diastereomers and enantiomers thereof,

wherein

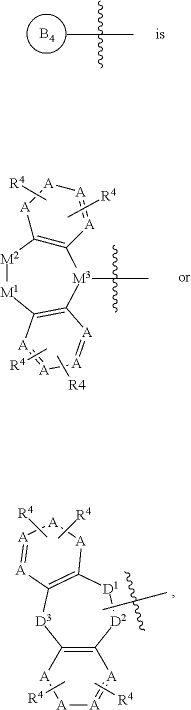

Q¹ is selected from the group consisting of —C₁-C₆alkyl, covalent bond, —C₀-C₆alkyl-O—C₀-C₆alkyl-, —C₀-C₆alkyl-NR₃—C₀-C₆alkyl-, —C₀-C₆alkyl-S(O)₀₋₂—C₀-C₆alkyl-, —C₀-C₆alkyl-NR₃C(O)—C₀-C₆alkyl-, —C₀-C₆alkyl-C(O)NR₃—C₀-C₆alkyl- and —C₀-C₆alkyl-OC(O)NR₃—C₀-C₆alkyl-; and

R³, R⁴, M¹-M², M³, A, D¹-D², D³ are as defined in Formula I.

In a preferred embodiment of embodiment XX, embodiment XX-1, the moiety

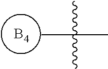

is selected from a radical consisting of

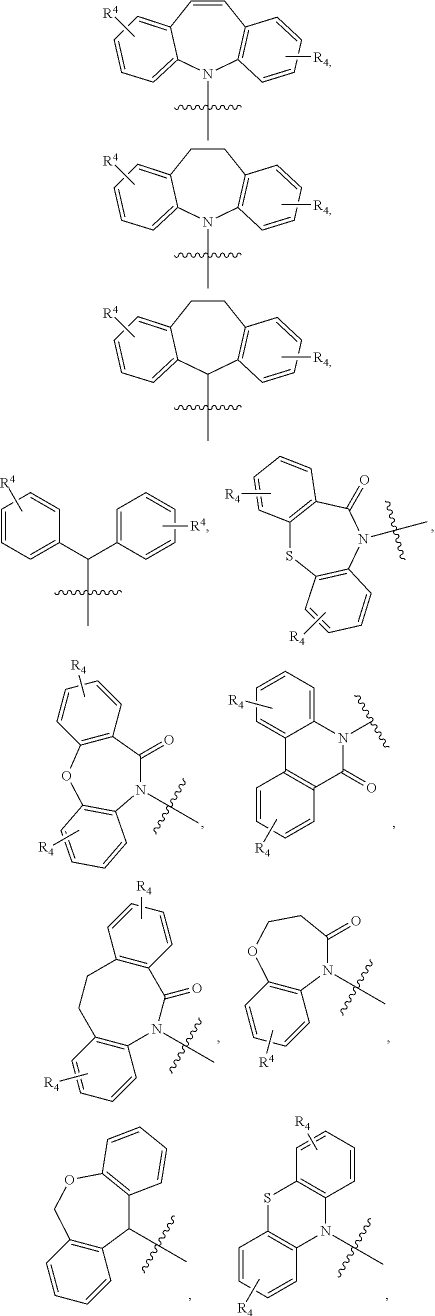

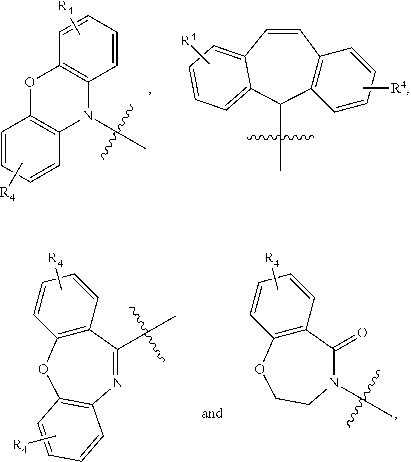

wherein R⁴ is as defined in Formula I.
In a preferred embodiment, embodiment YY, of the FTLD targeted agents according to the present disclosure, the compounds are represented by the Formula (XII):

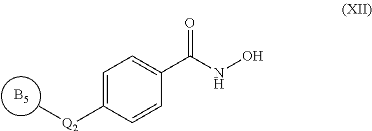

and N-oxides, hydrates, solvates, pharmaceutically acceptable salts, prodrugs, polymorphs and complexes thereof, and racemic and scalemic mixtures, diastereomers and enantiomers thereof, wherein

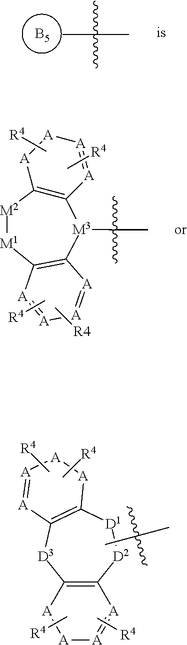

Q² is selected from the group consisting of —C₁-C₆alkyl, covalent bond, —C₀-C₆alkyl-O—C₀-C₆alkyl-, —C₀-C₆alkyl-NR₃—C₀-C₆alkyl-, —C₀-C₆alkyl-S(O)₀₋₂—C₀-C₆alkyl-, —C₀-C₆alkyl-NR₃C(O)—C₀-C₆alkyl-, —C₀-C₆alkyl-C(O)NR₃—C₀-C₆alkyl- and —C₀-C₆alkyl-OC(O)NR₃—C₀-C₆alkyl-; and

R³, R⁴, M¹-M², M³, A, D¹-D², D³ are as defined in Formula I;

In a preferred embodiment of embodiment YY, embodiment YY-1, the moiety

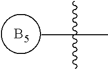

is selected from a radical consisting of

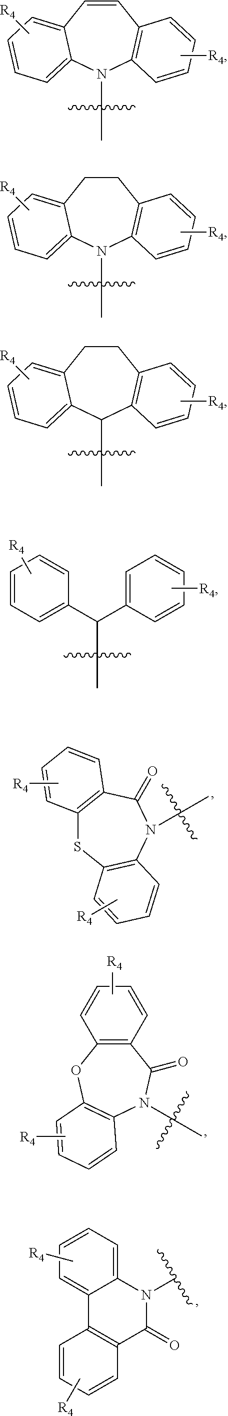

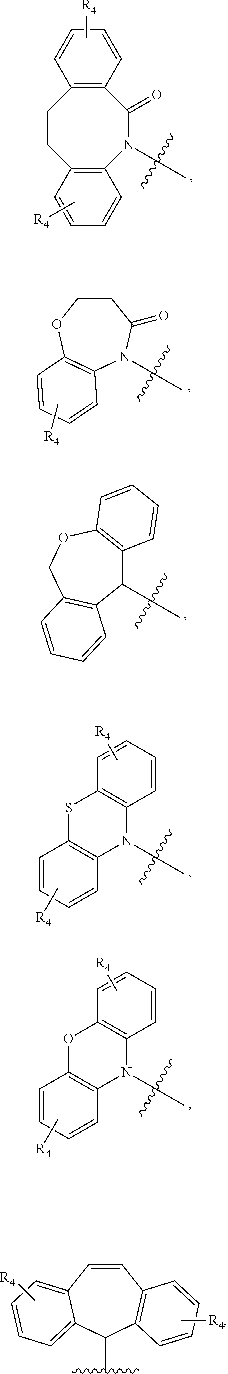

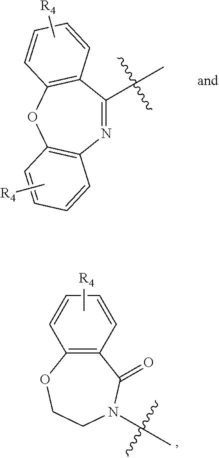

wherein R⁴ is as defined in Formula I.

In a preferred embodiment, embodiment ZZ, of the FTLD targeted agents according to the present disclosure, the compounds are represented by the Formula (XIII):

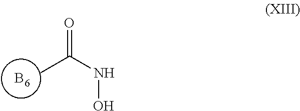

and N-oxides, hydrates, solvates, pharmaceutically acceptable salts, prodrugs, polymorphs and complexes thereof, and racemic and scalemic mixtures, diastereomers and enantiomers thereof, wherein

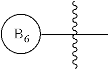

is a radical selected from the group consisting of

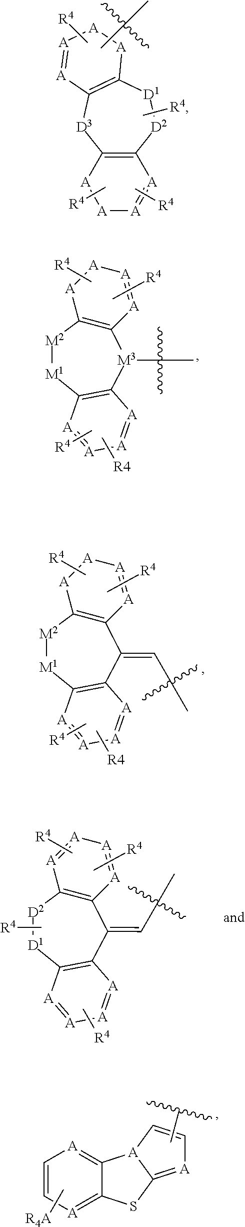

R⁴, M¹-M², M³, A, D¹-D², D³ are as defined in Formula I.

In a preferred embodiment, embodiment AAA, of the FTLD targeted agents according to the present disclosure, the compounds are represent by the Formula (XIV):

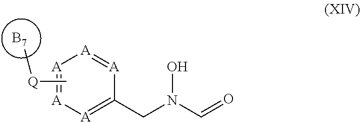

and N-oxides, hydrates, solvates, pharmaceutically acceptable salts, prodrugs, polymorphs and complexes thereof, and racemic and scalemic mixtures, diastereomers and enantiomers thereof,

wherein

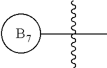

is a radical selected from the group consisting of aryl, heteroaryl, heterocyclyl, cycloalkyl,

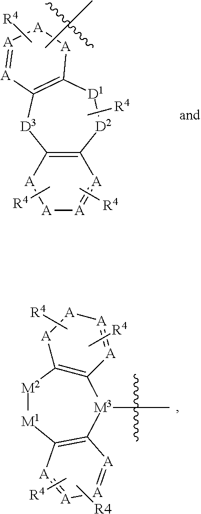

wherein each aryl, heteroaryl, cycloalkyl and heterocyclyl is optionally substituted; and

wherein Q, R⁴, M¹-M², M³, A, D¹-D², D³ are as defined in Formula I.

### Additional Compound Forms:

In the case compounds of Formula (I-IX) may contain asymmetric centers and exist as different enantiomers or diastereomers. All enantiomers or diastereomeric forms are embodied herein. The compounds of the invention may be racemic, or in a single enantiomer form

Compounds in the disclosure, e.g., FTLD targeted agents, may be in the form of pharmaceutically acceptable salts. The phrase “pharmaceutically acceptable” refers to salts prepared from pharmaceutically acceptable non-toxic bases and acids, including inorganic and organic bases and inorganic and organic acids. Salts derived from inorganic bases include lithium, sodium, potassium, magnesium, calcium and zinc. Salts derived from organic bases include ammonia, primary (e.g. Tromethamine), secondary and tertiary amines, and amino acids (e.g. Lysine). Salts derived from inorganic acids include sulfuric, hydrochloric, phosphoric, methanesulphonic, hydrobromic. Salts derived from organic acids include C₁₋₆ alkyl carboxylic acids, di-carboxylic acids and tricarboxylic acids such as acetic acid, propionic acid, fumaric acid, maleic acid, succinic acid, tartaric acid, adipic acid and citric acid, and alkylsulfonic acids such as methanesulphonic, and aryl sulfonic acids such as para-tolouene sulfonic acid and benzene sulfonic acid. For detailed list of slats see P. H. Stahl and C. G. Wermuth (eds.) “Handbook of Pharmaceutical Salts, Properties, Selection and Use” Wiley-VCH (ISBN 3-906390-26-8)

Compounds and pharmaceutically acceptable salts thereof may be in the form of a solvates. This occurs when a compound of the invention crystallizes in a manner that it incorporates solvent molecules into the crystal lattice. Examples of solvents forming solvates are water (hydrates), MeOH, EtOH, iPrOH, and acetone. Compounds of the invention described herein cover all solvates of the depicted compounds.

Compounds in the disclosure may exist in different crystal forms known as polymorphs.

Practitioners of the art will recognize that certain chemical groups may exist in multiple tautomeric forms. The scope of this disclosure is meant to include all such tautomeric forms. For example, a tetrazole may exist in two tautomeric forms, 1-H tetrazole and a 2-H tetrazole. This is depicted in figure below. This example is not meant to be limiting in the scope of tautomeric forms.

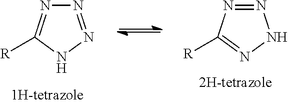

Practitioners of the art will recognize that certain electrophilic ketones, may exist in a hydrated form. The scope of this disclosure is to include all such hydrated forms. For example, a trifluoromethyl ketone may exist in a hydrated form via addition of water to the carbonyl group. This is depicted in figure below. This example is not meant to be limiting in the scope of hydrated forms.

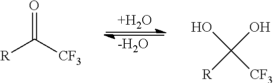

### Synthesis of Compounds of the Invention

In certain embodiments, the FTLD targeted agents may be prepared and/or substituted in the manner described in PCT/US2009/042818 filed on May 5, 2009 and published on Nov. 12, 2009 as WO/2009/137462.

### III. METHODS OF THE INVENTION

In another embodiment, the invention provides a method for targeted treatment of FTD or FTLD in a subject (e.g., a mammal, e.g., a human), wherein said method comprises administering an FTLD targeted agent to a subject identified as suffering from FTD or FTLD, such that the FTD or FTLD is treated in the subject.

In certain embodiments of the invention, the subject identified as suffering from FTD or FTLD is identified by an FTD or FTLD diagnostic assay. Alternatively, such assessment may be made without an FTD or FTLD diagnostic assay by a clinician qualified in the field of neurodegenerative disorders to assess frontotemporal lobe dementia in a subject, with an expectation that frontotemporal lobe dementia has high correlation to quantifiable values of FTLD.

In certain embodiments of the invention, the method for targeted treatment of FTD or FTLD may comprise an additional step of identifying the subject suffering from FTD or FTLD by administering to the subject an FTD or FTLD diagnostic assay.

The FTLD diagnostic assay serves as a quantifiable analysis tool in determining whether a subject may be identified with FrontoTemporal Lobar Degeneration (FTLD). In certain embodiments, the FTLD diagnostic assay identifies a mutant allele of the progranulin gene, wherein the presence of the mutant allele of the progranulin gene identifies the subject suffering from FTLD. In particular embodiments, the mutant allele of the progranulin gene is a mutant T allele of rs5848.

The methods and materials provided herein can be used to determine whether both alleles containing GRN nucleic acid of a mammal contain the mutant ‘T’ allele of rs5848, or whether only a single allele containing GRN nucleic acid of the mammal contains the mutant ‘T’ allele of rs5848. For example, this description provides methods and materials for determining whether or not a mammal is homozygous or heterozygous for the mutant ‘T’ allele of rs5848. As described herein, a subject that is homozygous or, in some cases heterozygous, for the mutant ‘T’ allele of rs5848 is identified as suffering from FTLD.

Any appropriate method can be used to detect the mutant ‘T’ allele of rs5848 in GRN nucleic acid. For example, mutations can be detected by sequencing cDNA, untranslated sequences, denaturing high performance liquid chromatography (DHPLC; underfill et al., Genome Res., 7:996-1005 (1997)), allele-specific hybridization (Stoneking et al., Am. J. Hum. Genet., 48:370-382 (1991); and Prince et al., Genome Res., 11(1): 152-162 (2001)), allele-specific restriction digests, mutation specific polymerase chain reactions, single-stranded conformational polymorphism detection (Schafer et al., Nat. Biotechnol., 15:33-39 (1998)), infrared matrix-assisted laser desorption/ionization mass spectrometry (WO 99/57318), and combinations of such methods.

In certain embodiments, genomic DNA can be used to detect the mutant ‘T’ allele of rs5848 in GRN nucleic acid. Genomic DNA typically is extracted from a biological sample such as a peripheral blood sample, but can be extracted from other biological samples, including tissues (e.g., mucosal scrapings of the lining of the mouth or from renal or hepatic tissue). Any appropriate method can be used to extract genomic DNA from a blood or tissue sample, including, for example, phenol extraction. In some cases, genomic DNA can be extracted with kits such as the QIAamp® Tissue Kit (Qiagen, Chatsworth, Calif.), the Wizard® Genomic DNA purification kit (Promega, Madison, Wis.), the Puregene DNA Isolation System (Gentra Systems, Minneapolis, Minn.), or the A.S.A.P.3 Genomic DNA isolation kit (Boehringer Mannheim, Indianapolis, Ind.).

An amplification step can be performed before proceeding with the detection method. For example, the 3′ UTR of a GRN nucleic acid can be amplified and then directly sequenced. Dye primer sequencing can be used to increase the accuracy of detecting heterozygous samples.

The mammal can be any type of mammal including, without limitation, a mouse, rat, dog, cat, horse, sheep, goat, cow, pig, monkey, or human. Examples of GRN nucleic acid include, without limitation, the nucleic acid sequence set forth in GenBank® Accession Number M75161 (GI:183612).

The present invention also relates to methods and materials for detecting mutations that are linked to frontotemporal lobe dementia. The methods and materials provided herein are based, in part, on the discovery that mutations within progranulin (GRN) nucleic acid are linked to frontotemporal lobe dementia (e.g., FTLD). The human GRN gene is located at chromosome 17q21, and its coding sequence is available at GenBank® Accession Number M75161 (g.i.:183612). The GRN gene is also known as epithelin precursor, proepithelin, PEPI, acrogranin, and granulin. A GRN gene can have 12 exons that together can encode a polypeptide with a molecular weight of 68.5 kDa. Granulins form a family of cysteine-rich polypeptides, some of which have growth modulatory activity. The widespread occurrence of GRN mRNA in cells from the hematopoietic system and in epithelia implies functions in these tissues. At least four different human granulin polypeptides can be processed from a single GRN precursor which can contain 7.5 repeats that each contain 12 conserved cysteine residues. Both the GRN precursor and processed GRN polypeptides can have biological activity. The term “GRN polypeptide” as used herein includes, without limitation, human GRN polypeptides (e.g., human GRN polypeptides set forth in GenBank® under g.i. numbers 183612, 4504151, and 77416865). A human progranulin polypeptide can be a 593-amino acid glycosylated polypeptide having a consensus sequence that is repeated seven and a half times. Additional exemplary mutations that may be used in the diagnostic assay include, but are not limited to those described in Human Molecular Genetics, 2006, Vol. 15, No. 20 2988-3001, Mutations in progranulin are a major cause of ubiquitin-positive frontotemporal lobar degeneration by Gass et al., and in NATURE|Vol 442|24 Aug. 2006, Mutations in progranulin cause tau-negative frontotemporal dementia linked to chromosome 17 by Baker et al.

Alternatively, the FTLD diagnostic assay measures progranulin or progranulin mRNA levels. The skilled artisan may measure these levels, in light of the present invention, in a variety of acceptable ways. However, an exemplary method of analysis of these levels is depicted the Exemplification section. For example, loss-of-function GRN mutations demonstrate a significant reduction in progranulin levels. Such loss-of-function mutations, in certain embodiments, measure to be about one third of the level observed in wild type. GRN levels may range from 53 to 94 ng/ml (mean value±SD: 68±16 ng/ml) in mutation carriers, while non-GRN carriers show levels from 115 to 386 ng/ml (mean value±SD: 220±47 ng/ml).

In certain embodiments, the compounds of the invention also show a positive effect on cognitive and memory performance.

In another embodiment, the invention provides a method for treating frontotemporal lobe dementia in a subject, wherein said method comprises administering an FTLD targeted agent to a subject identified as suffering from FTLD, such that the frontotemporal lobe dementia is treated in the subject. The diagnosis of a subject with frontotemporal lobe dementia may be clinically confirmed by measurement and analysis of progranullin levels or progranulin mRNA levels. Symptoms of dementia can include changes in behavior such as changes that result in impulsive, repetitive, compulsive, or even criminal behavior. For example, changes in dietary habits and personal hygiene can be symptoms of dementia. Symptoms of dementia also can include language dysfunction, which can present as problems in expression of language, such as problems using the correct words, naming objects, or expressing oneself. Difficulties reading and writing can also develop.

In yet another embodiment, the invention provides a method for treating FrontoTemporal Lobar Degeneration (FTLD) in a subject identified by an FTLD diagnostic assay, wherein said method comprises identifying a subject suffering from FTLD by applying to the subject an FTLD diagnostic assay, and administering to said identified subject an FTLD targeted agent, such that the FTLD is treated in a subject.

The FLTD targeted agent may comprise one or more of the compounds described in the Compounds of the Invention section.

For example, in certain embodiments, the FTLD targeted agent has Formula (IV):

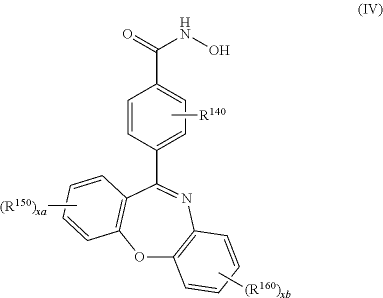

wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xa and xb denote numbers that are each independently selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, C₀-C₆-alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiments of the invention, the FTLD targeted agent has Formula (V):

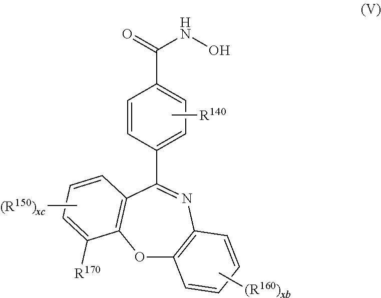

wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xb denotes a number selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xc is 0 or 1; and
R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —CO—C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
   or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl; and
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

In certain embodiments of the invention, the FTLD targeted agent has Formula (VI):

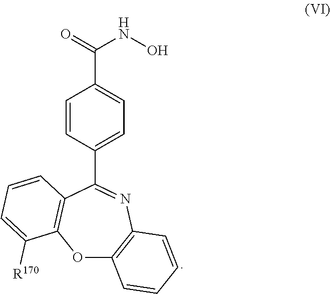

In certain embodiments of the invention, the FTLD targeted agent is:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide, and
(Z)—N-hydroxy-4-(5-(2-morpholinoethyl)-5H-dibenzo[b,e][1,4]diazepin-11-yl)benzamide or a pharmaceutically acceptable salt thereof.

### IV. PHARMACEUTICAL COMPOSITIONS OF THE INVENTION

Another embodiment of the invention provides pharmaceutical compositions for targeted treatment of FTLD in a subject comprising a therapeutically effective amount of a compound of the invention, a derivative or a pharmaceutically acceptable salt thereof, and a pharmaceutically acceptable excipient, carrier or diluent.

In a particular embodiment, the invention provides a diminished peripheral formulation comprising an FTLD targeted agent (e.g., a compound of the invention), and a pharmaceutically acceptable carrier, wherein the FTLD targeted agent is formulated to improve the targeted treatment of FTLD. In certain embodiments, this formulation is suited to increase brain penetration and/or reduce peripheral dose levels.

The pharmaceutical compositions or formulations can be administered in a variety of dosage forms including, but not limited to, a solid dosage form or in a liquid dosage form, an oral dosage form, a parenteral dosage form, an intranasal dosage form, a suppository, a lozenge, a troche, buccal, a controlled release dosage form, a pulsed release dosage form, an immediate release dosage form, an intravenous solution, a suspension or combinations thereof. The dosage can be an oral dosage form that is a controlled release dosage form. The oral dosage form can be a tablet or a caplet. The compounds can be administered, for example, by oral or parenteral routes, including intravenous, intramuscular, intraperitoneal, subcutaneous, transdermal, airway (aerosol), rectal, vaginal and topical (including buccal and sublingual) administration. In one embodiment, the compounds or pharmaceutical compositions comprising the compounds are delivered to a desired site, such as the brain, by continuous injection via a shunt.

In another embodiment, the compound can be administered parenterally, such as intravenous (i.v.) administration. The formulations for administration will commonly comprise a solution of the compound of the invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of compound of the invention in these formulations can vary widely, and will be selected primarily based on fluid volumes, viscosities, body weight, and the like, in accordance with the particular mode of administration selected and the patient's needs. For i.v. administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In one embodiment, a compound of the invention can be administered by introduction into the central nervous system of the subject, e.g., into the cerebrospinal fluid of the subject. The formulations for administration will commonly comprise a solution of the compound of the invention dissolved in a pharmaceutically acceptable carrier. In certain aspects, the compound of the invention is introduced intrathecally, e.g., into a cerebral ventricle, the lumbar area, or the cisterna magna. In another aspect, the compound of the invention is introduced intraocularly, to thereby contact retinal ganglion cells.

The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

In one embodiment, the pharmaceutical composition comprising a compound of the invention is administered into a subject intrathecally. As used herein, the term “intrathecal administration” is intended to include delivering a pharmaceutical composition comprising a compound of the invention directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a compound of the invention to any of the above mentioned sites can be achieved by direct injection of the pharmaceutical composition comprising the compound of the invention or by the use of infusion pumps. For injection, the pharmaceutical compositions can be formulated in liquid solutions, preferably in physiologically compatible buffers such as Hank's solution or Ringer's solution. In addition, the pharmaceutical compositions may be formulated in solid form and re-dissolved or suspended immediately prior to use. Lyophilized forms are also included. The injection can be, for example, in the form of a bolus injection or continuous infusion (e.g., using infusion pumps) of pharmaceutical composition.

In one embodiment, the pharmaceutical composition comprising a compound of the invention is administered by lateral cerebro ventricular injection into the brain of a subject. The injection can be made, for example, through a burr hole made in the subject's skull. In another embodiment, the encapsulated therapeutic agent is administered through a surgically inserted shunt into the cerebral ventricle of a subject. For example, the injection can be made into the lateral ventricles, which are larger, even though injection into the third and fourth smaller ventricles can also be made.

In yet another embodiment, the pharmaceutical composition is administered by injection into the cisterna magna, or lumbar area of a subject.

For oral administration, the compounds will generally be provided in unit dosage forms of a tablet, pill, dragee, lozenge or capsule; as a powder or granules; or as an aqueous solution, suspension, liquid, gels, syrup, slurry, etc. suitable for ingestion by the patient. Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutical preparations for oral use can be obtained through combination of a compound of the invention with a solid excipient, optionally grinding a resulting mixture, and processing the mixture of granules, after adding suitable additional compounds, if desired, to obtain tablets or dragee cores. Suitable solid excipients in addition to those previously mentioned are carbohydrate or protein fillers that include, but are not limited to, sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For transmucosal administration (e.g., buccal, rectal, nasal, ocular, etc.), penetrants appropriate to the barrier to be permeated are used in the formulation. Such penetrants are generally known in the art.

Formulations for rectal administration may be presented as a suppository with a suitable base comprising for example cocoa butter or a salicylate. Formulations suitable for vaginal administration may be presented as pessaries, tampons, creams, gels, pastes, foams or spray formulations containing in addition to the active ingredient such carriers as are known in the art to be appropriate. For intramuscular, intraperitoneal, subcutaneous and intravenous use, the compounds will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The suppositories for rectal administration of the drug can be prepared by mixing the drug with a suitable non-irritating excipient which is solid at ordinary temperatures but liquid at the rectal temperatures and will therefore melt in the rectum to release the drug. Such materials are cocoa butter and polyethylene glycols.

The compounds can be delivered transdermally, by a topical route, formulated as applicator sticks, solutions, suspensions, emulsions, gels, creams, ointments, pastes, jellies, paints, powders, or aerosols.

The compounds may also be presented as aqueous or liposome formulations. Aqueous suspensions can contain a compound of the invention in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil suspensions can be formulated by suspending a compound of the invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In addition to the formulations described previously, the compounds may also be formulated as a depot preparation. Such long acting formulations may be administered by implantation or transcutaneous delivery (e.g., subcutaneously or intramuscularly), intramuscular injection or a transdermal patch. Thus, for example, the compounds may be formulated with suitable polymeric or hydrophobic materials (e.g., as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

For administration by inhalation, the compounds are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In general a suitable dose will be in the range of 0.01 to 100 mg per kilogram body weight of the recipient per day, preferably in the range of 0.2 to 10 mg per kilogram body weight per day. The desired dose is preferably presented once daily, but may be dosed as two, three, four, five, six or more sub-doses administered at appropriate intervals throughout the day.

The compounds can be administered as the sole active agent, or in combination with other known therapeutics to be beneficial in the treatment of neurological disorders. In any event, the administering physician can provide a method of treatment that is prophylactic or therapeutic by adjusting the amount and timing of drug administration on the basis of observations of one or more symptoms (e.g., motor or cognitive function as measured by standard clinical scales or assessments) of the disorder being treated.

Details on techniques for formulation and administration are well described in the scientific and patent literature, see, e.g., the latest edition of Remington's Pharmaceutical Sciences, Maack Publishing Co, Easton Pa. (“Remington's After a pharmaceutical composition has been formulated in an acceptable carrier, it can be placed in an appropriate container and labeled for treatment of an indicated condition. For administration of the compounds of the invention, such labeling would include, e.g., instructions concerning the amount, frequency and method of administration.

The present invention is illustrated by the following examples, which are not intended to be limiting in any way.

### Example 1

### General Measurement of Gene Expression

Primary cortical (Cx) and hippocampal (Hp) neurons are derived from E17 Sprague-Dawley rats and cultured in 24-well plates (325,000 cells/well for Cx and 250,000 cells/well for Hp). Half of the medium is replaced with fresh medium every four days. On day 10 of culture, neurons are incubated in the presence of different concentrations (0.1-3 μM) of a compound of the invention, e.g., Compound 1, for 2-24 h. At specific time points, conditioned medium is removed and frozen until needed for ELISA assay. Cells are washed with PBS and RNA is extracted with Qiazol reagent using the RNeasy kit from Qiagen (Valencia, Calif.) following manufacturer instructions. RNA is then transcribed into cDNA using the high capacity cDNA reverse transcription kit from Applied Biosystems (Carlsbad, Calif.) according to kit instructions.

Relative progranulin gene expression is measured by quantitative PCR using rat-specific probes (Applied Biosystems, Carlsbad, Calif.) for the granulin gene and for Rp113a as a reference gene. The ΔΔCt method is used for calculations of relative gene expression levels (Pfaffl, Nucleic Acids Res., 2001). This method compares expression of the different genes (reference and gene of interest) in cells treated with vehicle versus the compound of the invention. Moreover, this measurement and analysis of mRNA levels is expected to be predictive of the expression of progranulin protein levels.

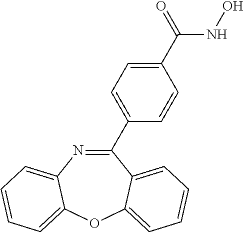 [SMILES: O=C(NO)c1ccc(C2=Nc3ccccc3Oc3ccccc32)cc1]

### (Z)-4-(dibenzo[b,f][1,4]oxazepin-11-yl)-N-hydroxybenzamide (Compound 1)

Progranulin protein levels may be measured in the neuronal culture medium using an ELISA kit form Adipogen (Incheon, South Korea) according to manufacturer instructions.

### Example 2

### Dose/Time Dependent Progranulin Expression Levels

Neuronal granulin gene expression was measured at different time points as described in Example 1 (1, 4, 6, 8, 10, 18 and 24 h) following treatment with (Z)-4-(dibenzo[b,f][1,4]oxazepin-11-yl)-N-hydroxybenzamide

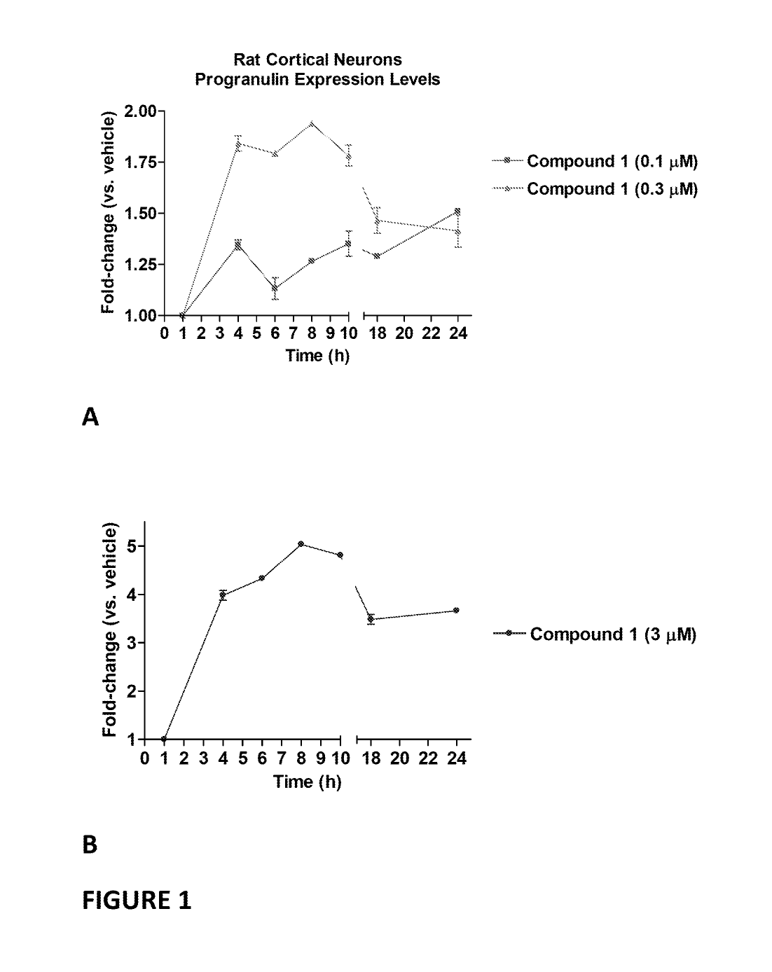

(Compound 1) at 0.1, 0.3 and 3 μM. Compound 1 increased progranulin mRNA expression in a dose and time-dependent manner, reaching increases of ˜2-fold and ˜5-fold after 8 h of treatment with 0.3 μM and 3 μM respectively as shown in FIGS. 1A and 1B.

With the lowest dose, ˜40% increase in progranulin gene expression was observed as soon as 4 h after treatment and was sustained for 24 h.

Given the results obtained in normal cells, Compound 1 is expected to induce progranulin expression in models of Frontotemporal dementia linked to progranulin deficiency.

Confirmation is expected to be established through investigation of the induction of progranulin by Compound 1 in cellular models carrying heterozygous human progranulin mutations that lead to haploinsufficiency and ultimately, frontotemporal dementia. Such analysis includes examining the effects of Compound 1 on progranulin levels in brain cortex and hippocampus, CSF, and plasma, as well as other tissues and brain areas, in mouse models carrying disease-causing alteration of the progranulin gene (mutation or knockout of one allele).

### Example 3

### Comparative Analysis of Brain Penetration of HDAC Inhibitors

Compound 1, e.g., a representative FTLD targeted agent of the invention, displays high brain penetration in rodents after acute and repeated dosing. The brain:plasma (b:p) ratios for drug levels observed are ˜5 for Compound 1, demonstrating excellent brain penetration.

In contrast, SAHA (Zolinza), a HDACi approved for use in cancer patients displays a b:p of approximately 0.1 in acute dosing experiments in rodents. Furthermore, a ˜30 fold excess of plasma protein unbound (‘free’) SAHA is required to produce acetylation of histones in the brain of mice, compared to Compound 1. This means that the exposure of SAHA needs to be ˜30 times higher in the periphery of the body to achieve the same effect on acetylation of histones that Compound 1 achieves in the brain. Because target histone deacetylases are expressed throughout the body, this will result in increased peripheral on-target toxicity of SAHA compared to Compound 1. In addition, ˜30fold higher peripheral off-target toxicity can be expected.

In humans, dosing of SAHA and Compound 1 with 400 mg orally would be expected to result in plasma drug exposures that are roughly equivalent. However, because 400 mg is the Maximum Tolerated Dose (MTD) of SAHA in cancer patients, whereas an MTD was not observed with Compound 1 at 400 mg in healthy volunteers (the highest dose used), the use of SAHA to achieve increased levels of brain exposure is expected to be restricted in humans due to its side effects, while the compounds of the invention have enhanced utility as targeted treatment for FTLD.

### Example 4

### Dose/Time-Dependent Progranulin Expression Levels in Patient Lymphoblasts

Immortalized (Epstein-Barr-Virus) B-lymphocytes isolated from the peripheral blood of patients subject harboring different mutations in the granulin gene were used to measure effects of compounds on progranulin mRNA and protein expression. The cell lines were maintained in complete growth medium (cGM), comprised of RPMI 1640 medium supplemented with 15% heat-inactivated FBS, 4 mM L-Glutamine and 1% Penicillin/Streptomycin. Cells were seeded into 24-well tissue-culture plates at a density of 400,000-500,000 cells per well in cGM. Following an overnight incubation, the cells were treated with 0.3 or 3 μM of Compound 1. Following an 8- or 24-hour exposure to Compound 1, cells were collected, spun, and the resulting cell pellets underwent a single rinse in chilled PBS.

For analysis of progranulin mRNA expression, samples were analyzed as described in Example 1, with the exception that probes specific for the human granulin gene (Applied Biosystems, Carlsbad, Calif.) were used.

For analysis of progranulin protein expression, cell pellets were re-suspended in 150 μl of chilled RIPA Lysis Buffer supplemented with a protease-inhibitor cocktail and incubated for 15-20 minutes at 4° C. to allow for cell lysis. Total protein concentrations of each sample were determined by performing a BCA protein assay (Pierce). Quantitative determination of Progranulin protein levels was performed by ELISA (AdipoGen, Incheon, South Korea), using a 1:20 dilution of the cell lysate following the manufacturer protocol. Progranulin levels were expressed relative to protein amount, and compound treatment groups were expressed relative to the vehicle-treated group.

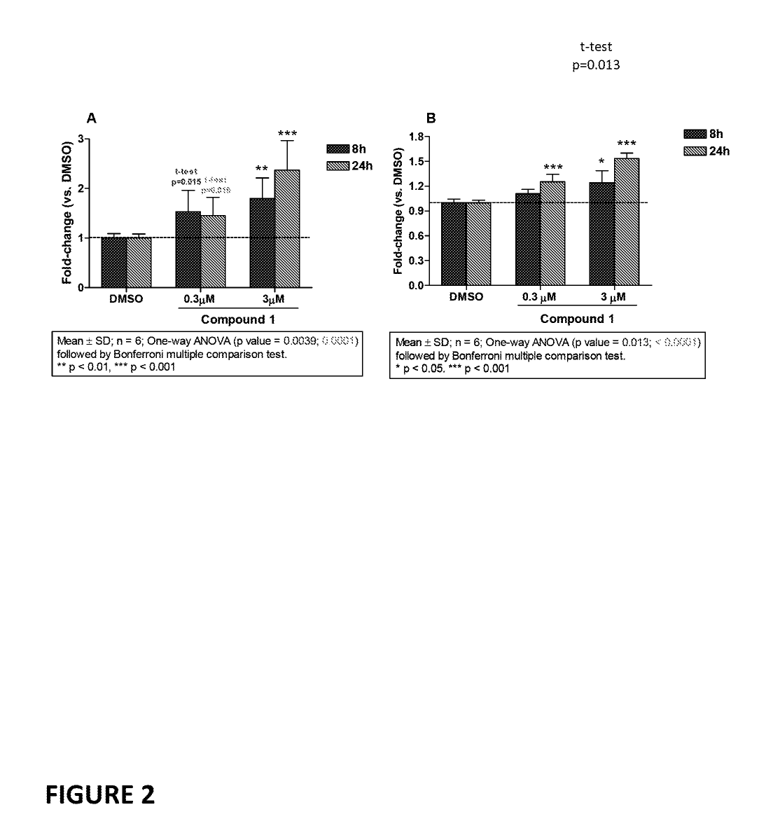

In lymphoblasts from progranulin mutation carriers, Compound 1 induced an increase in progranulin mRNA in a dose and time-dependent manner, reaching increases of 2.4-fold after 24 h with 3 μM (FIG. 2A). This increase also translated to proganulin protein expression, albeit to a lesser extent. The increase reached up to 1.5-fold after 24 h with 3 μM of Compound 1 (FIG. 2B).

### Example 5

### Dose/Time-Dependent Progranulin Expression Levels in Patient Fibroblasts

Primary fibroblasts from patients harboring different mutations in the progranulin gene were isolated and maintained in complete growth medium (cGM), comprised of D-MEM supplemented with 10% heat-inactivated FBS and 1% Penicillin/Streptomycin. The fibroblasts were seeded into 6-well tissue-culture plates at a density of 150,000-190,000 cells per well in cGM. Following overnight incubation, the cells were treated with 0.3 or 3 μM of Compound 1. Analysis of progranulin mRNA expression was performed as described in Example 4. Analysis of progranulin protein was performed as described in Example 4.

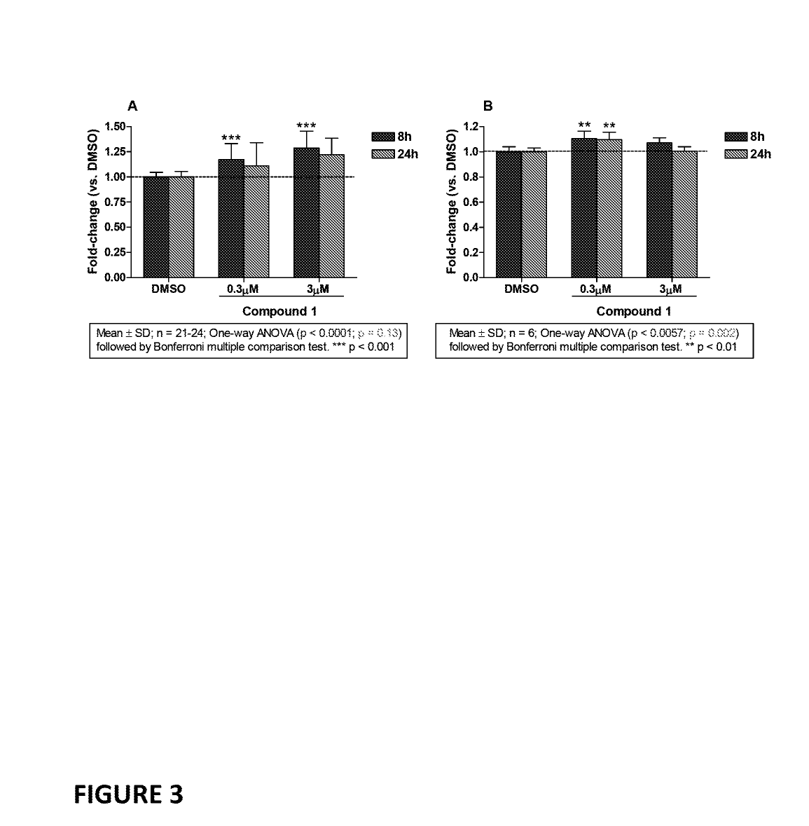

In primary fibroblasts from progranulin mutation carriers, Compound 1 induced an increase in progranulin mRNA in a dose and time-dependent manner, reaching increases of 1.3-fold after 8 h with 3 μM (FIG. 3A). This increase also translated to progranulin protein expression, albeit to a lesser extent. The increase reached up to 1.1-fold after 8 h with 0.3 μM of Compound 1 (FIG. 3B).

### Example 6

### Dose Dependent Progranulin Expression Levels in Human Lymphoblasts

Cellular protein from immortalized (Epstein-Barr-Virus) B-lymphocytes isolated from the peripheral blood of normal human volunteers was isolated as described in Example 4. Cells were treated with Compound 1 for 24 h.

For Western Blotting analysis of progranulin protein, 50 μg of total cellular protein were separated on a 4-12% Bis-Tris Poly-Acrylamide gel using MOPS Running Buffer, transferred to a PVDF membrane and probed with a R & D Systems (Minneapolis, Minn.) mouse monoclonal anti-human progranulin antibody, followed by an IRDye secondary antibody (Rockland). Detection was performed using the Li-COR Odyssey imaging system. Progranulin signals (Integrated Intensity after background subtraction) were quantified using Li-COR Odyssey software.

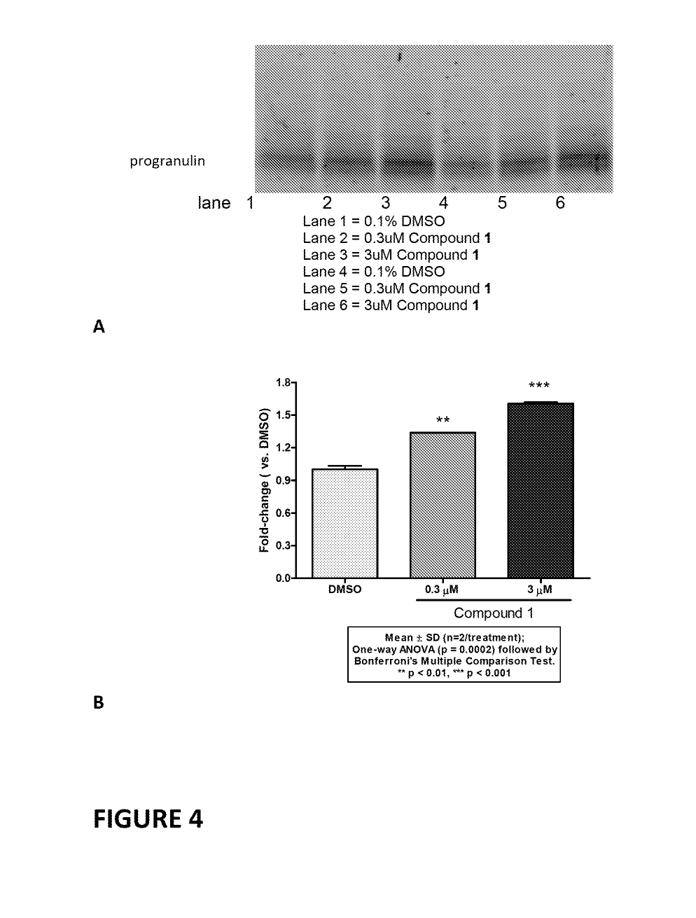

In immortalized lymphoblasts from a normal human subject, Compound 1 induced an increase in progranulin protein as measured by western blot (FIG. 4A). The increase was dose-dependent, reaching 1.3-fold for 0.3 μM and 1.6-fold for 3 μM (FIG. 4B).

### Example 7

### Progranulin Expression Levels in Mouse Brain

C57BL/6 mice (n=6/group; Charles River) were treated with either vehicle (1% carboxymethylcellulose-medium viscosity/0.5% Tween 80 (99.5:0.5 v/v)) or with an oral dose of 100 mg/kg of Compound 1 prepared in the same vehicle. Animals were sacrificed at specific time points and the brain extracted. Left and right hemisphere of the cerebral cortex were separated and used for RNA and protein extractions.

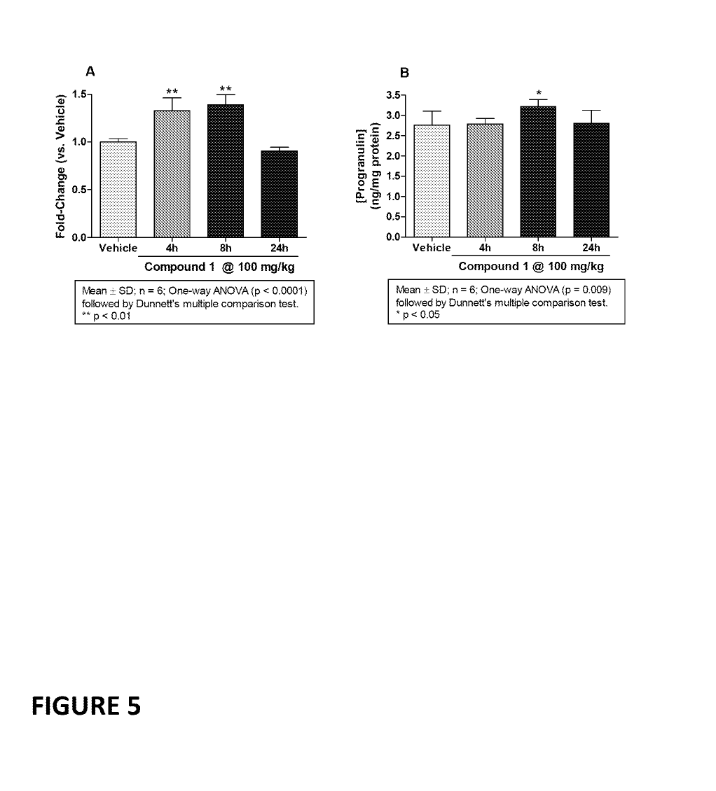

RNA was extracted with Qiazol reagent using the RNeasy lipid-rich tissue kit from Qiagen (Valencia, Calif.) following manufacturer instructions. RNA was then transcribed into cDNA using the high capacity cDNA reverse transcription kit from Applied Biosystems (Carlsbad, Calif.), according to kit instructions. Relative progranulin gene expression was measured by quantitative PCR using mouse-specific probes (Applied Biosystems, Carlsbad, Calif.) for the granulin gene, and for Rp113a as a reference gene. The ΔΔCt method was used for calculations of relative gene expression levels (Pfaffl, Nucleic Acids Res., 2001). This method compares expression of the different genes (reference and gene of interest) in animals treated with vehicle versus the compound of the invention (FIG. 5A).

Proteins were extracted in 50 mM Tris pH 7.4/150 mM NaCl/0.1% SDS with protease inhibitors using an extraction ratio of 200 mg of brain tissue per ml buffer. Sample was spun for 20 minutes at 16,200 g and supernatant collected. Progranulin levels were measured using a 1:4 dilution of the tissue extract (FIG. 5B) with a mouse ELISA kit from Adipogen (Incheon, South Korea) according to the manufacturer instructions. Progranulin levels were normalized to total protein concentration as measured by the BCA method (Pierce).

In the brain of normal mice, Compound 1 induced an increase in progranulin mRNA by 1.45-fold 8 h after acute dosing (FIG. 5A). Protein levels increased concomitantly by about 1.16-fold (FIG. 5B).

### Example 8

### Progranulin Expression Levels in Rat Plasma and CSF

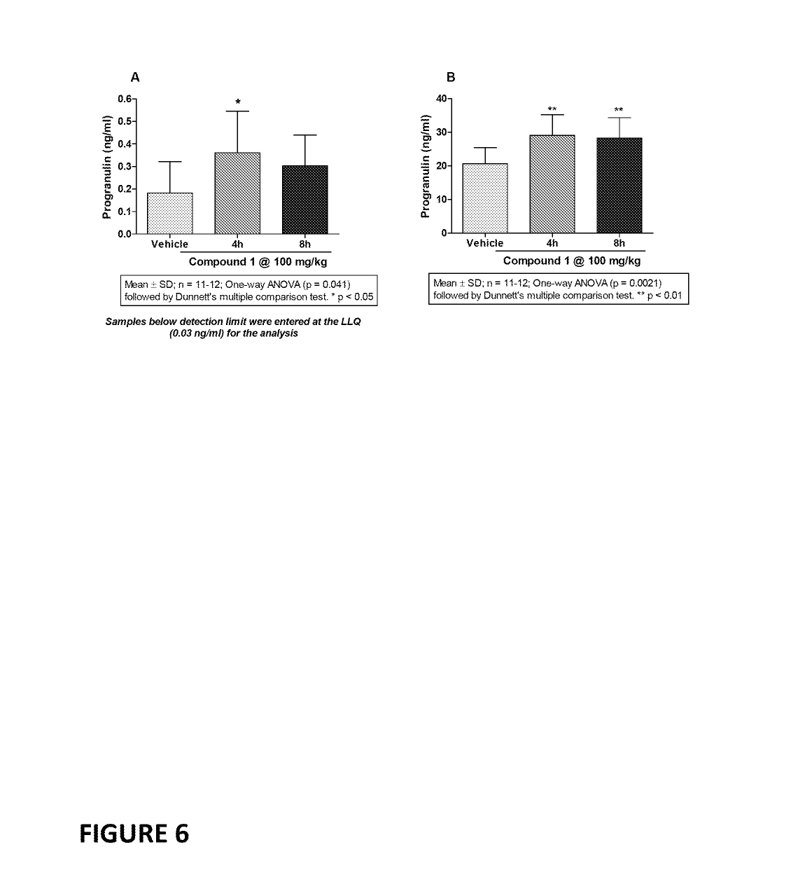

Sprague Dawley rats (n=12/group; Charles River) were dosed intra-peritoneally with either vehicle (Solutol HS 15/Ethanol (3:2 v/v)) or with a dose of 100 mg/kg of Compound 1 prepared in the same vehicle. EDTA-Plasma and CSF were collected after 4 and 8 hours of treatment, and progranulin levels were assessed by ELISA (Adipogen, Incheon, South Korea) using a 1:2 dilution for CSF (FIG. 6A) and a 1:25 dilution for plasma (FIG. 6B).

In the CSF and plasma of normal rats, Compound 1 induced an increase in CSF progranulin protein by about 1.9-fold 4 h after acute dosing. In plasma, progranulin protein levels increased concomitantly by about 1.4-fold.

### Example 9

### Dose/Time Progranulin Expression Levels in Rat Primary Cortical Neurons

Primary cortical neurons are prepared and treated as in Example 1. Total protein extraction, determination and progranulin levels measurement were performed as described in Example 4.

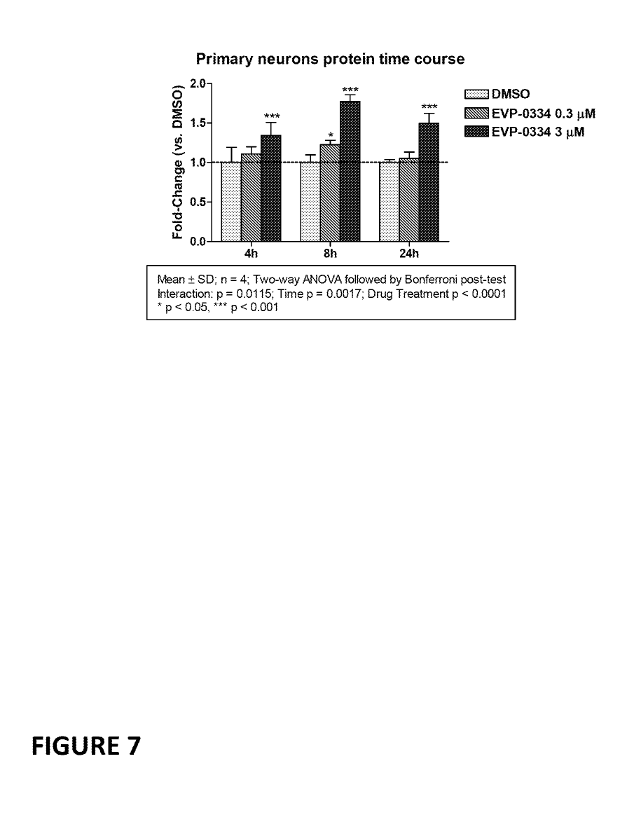

In rat primary cortical neurons, Compound 1 induced a dose and time-dependent increase in progranulin protein expression, which reached up to 1.77-fold after 8 h with 3 μM of Compound 1 (FIG. 7).

### Summary Conclusion Examples 1-9

Examples 4 and 5 demonstrate that Compound 1 increases progranulin protein and RNA levels significantly in cells from human FTLD-PGRN mutation carriers. Furthermore, examples included in examples 1-9 demonstrate that Compound 1 can increase progranulin levels in a variety of biological systems including cultured brain cells, blood cells, in vivo brain, in vivo CSF, and in vivo blood of human/rodent species examples. Combined, the examples show that Compound 1 could be useful for the treatment of FTLD-PGRN, with a potential to modify the disease and to alleviate symptoms.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

1. A method of treating frontotemporal dementia (FTD) or frontotemporal lobar degeneration (FTLD) comprising administering to a patient in need thereof an effective amount of a composition comprising a compound of Formula (IV): 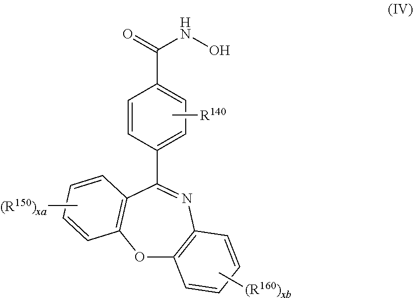
or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xa and xb denote numbers that are each independently selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

2. The method of claim 1, wherein the composition comprises a compound of Formula (V): 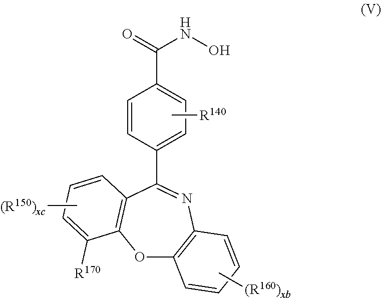
or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xb denotes a number selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xc is 0 or 1; and
R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl; and
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

3. The method of claim 2 wherein xb and xc are 0.

4. The method of claim 3 wherein R¹⁴⁰ is selected from the group consisting of: H, —OH, halo, —CN, —C₁-C₄alkyl, —C₁-C₄alkoxyl, —CF₃, —OCF₃, and —NO₂.

5. The method of claim 3 or 4 wherein R¹⁷⁰ is selected from: H, halo, —CN, —CF₃, —OCF₃, —C₁-C₆alkyl, and —C₁-C₆alkoxyl.

6. The method of claim 2 wherein the composition comprises a compound of Formula (VI): 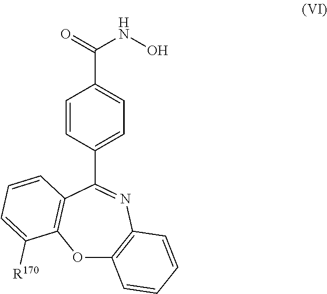
or a pharmaceutically acceptable salt thereof.

7. The method of claim 6 wherein R¹⁷⁰ is selected from: H, halo, —CN, —CF₃, —OCF₃, —C₁-C₆alkyl, and —C₁-C₆alkoxyl.

8. The method of claim 1 wherein the composition comprises a compound selected from:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide,
(Z)—N-hydroxy-4-(5-(2-morpholinoethyl)-5H-dibenzo[b,e][1,4]diazepin-11-yl)benzamide,
or a pharmaceutically acceptable salt thereof.

9. The method of claim 1 wherein the composition comprises (Z)-4-(dibenzo[b,f][1,4]oxazepin-11-yl)-N-hydroxybenzamide or a pharmaceutically acceptable salt thereof.

10. The method of claim 1 wherein the patient is suffering FTD.

11. The method of claim 1 wherein the patient is suffering from FTLD.

12. A method of treating a patient at risk of developing frontotemporal dementia (FTD) or frontotemporal lobar degeneration (FTLD), comprising administering to the patient an effective amount of pharmaceutical composition comprising a compound of Formula (IV): 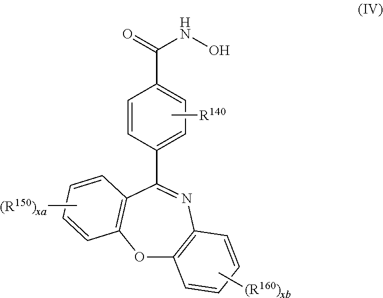
or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xa and xb denote numbers that are each independently selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

13. The method claim 12, wherein the composition comprises a compound of Formula (V): 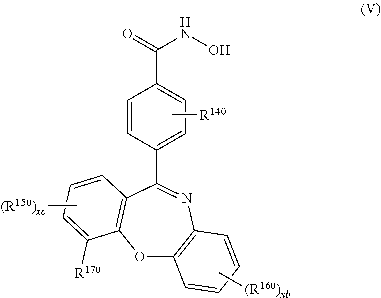
or a pharmaceutically acceptable salt thereof
wherein
R¹⁴⁰ is selected from the group consisting of H, —OH, halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xb denotes a number selected from 0, 1 and 2; and
R¹⁵⁰ and R¹⁶⁰ are independently selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₀-C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, and wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂;
xc is 0 or 1; and
R¹⁷⁰ is selected from the group consisting of H, halo, —CN, —CF₃, —OCF₃, —C₁-C₆alkyl, —C₁-C₆alkoxyl, —O—C₂-C₆alkyl-O—R⁵³, —OR⁵³, —C₀-C₆alkyl-S(O)₀₋₂—R⁵³, —C₀-C₆alkyl-C(O)—R⁵³, —C₀-C₆alkyl-C(O)NR⁵⁰R⁵¹, —C₀-C₆alkyl-NR⁵²C(O)—R⁵³, —C₀-C₆alkyl-S(O)₂NR⁵⁰R⁵¹, —C₀-C₆alkyl-NR⁵²S(O)₂—R⁵³, —C₀-C₆alkyl-OC(O)NR⁵⁰R⁵¹, —C₆alkyl-NR⁵²C(O)O—R⁵³, —C₀-C₆alkyl-NR⁵²C(O)NR⁵⁰R⁵¹, —C₀-C₆alkyl-C(O)O—R⁵³, —C₀-C₆alkyl-OC(O)—R⁵³, —C₀-C₆alkyl-aryl, —C₀-C₆alkyl-heteroaryl, —C₀-C₆alkyl-cycloalkyl, —C₀-C₆alkyl-heterocyclyl, —NH₂, —NR⁵⁰R⁵¹, —C₁-C₆alkyl-NR⁵⁰R⁵¹, —O—C₂-C₆alkyl-NR⁵⁰R⁵¹, —NR⁵³—C₂-C₆alkyl-NR⁵⁰R⁵¹ and —O-heterocyclyl-R⁵³, wherein each alkyl and heteroalkyl is optionally substituted with one or three substituents independently selected from the group consisting of F, —OH and oxo, wherein each aryl, heteroaryl, cycloalkyl and heterocyclyl is optionally substituted with one or two substituents independently selected from the group consisting of halo, —CN, —C₁-C₄alkyl, —C₁-C₄alkoxyl, —O—C₂-C₄alkyl-O—C₁-C₄alkyl, —CF₃, —OCF₃, —NO₂, —C₁-C₆alkyl-S(O)₀₋₂R⁵³, —NH₂, —NR⁵⁰R⁵¹, —C₁-C₆alkyl-NR⁵⁰R⁵¹ and —N(C₁-C₆alkyl)₂
R⁵⁰ and R⁵¹ are independently selected from the group consisting of H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl;
or
R⁵⁰ and R⁵¹, together with the N atom to which they are attached, optionally form a 3-10 membered heterocyclic ring, wherein the heterocyclyl is optionally substituted with one to three substituents independently selected from the group consisting of halo, —OH, amino, —CN or —C₁-C₄alkyl;
R⁵² is independently selected from the group consisting of —H, —C₁-C₆alkyl, —C₂-C₆alkyl-O—C₁-C₆alkyl, —C₀-C₆alkyl-C₃-C₇cycloalkyl, wherein each alkyl and cycloalkyl is optionally substituted with one or more substituents independently selected from the group consisting of halo, —OH, amino, —CN or —C₁-C₄alkyl; and
R⁵³ is independently selected from the group consisting of —C₁-C₆alkyl, —C₀-C₄alkyl-C₃-C₇cycloalkyl, —C₀-C₄alkyl-aryl, —C₀-C₄alkyl-heteroaryl and —C₀-C₄alkyl-heterocyclyl, wherein each alkyl, aryl, heteroaryl and heterocyclyl is optionally substituted with one or three substituents independently selected from the group consisting of halo, —OH, amino, —CN or —C₁-C₄alkyl.

14. The method of claim 13 wherein xb and xc are 0.

15. The method of claim 14 wherein R¹⁴⁰ is selected from the group consisting of: H, —OH, halo, —CN, —C₁-C₄alkyl, —C₁-C₄alkoxyl, —CF₃, —OCF₃, and —NO₂.

16. The method of claim 14 or 15 wherein R¹⁷⁰ is selected from: H, halo, —CN, —CF₃, —OCF₃, —C₁-C₆alkyl, and —C₁-C₆alkoxyl.

17. The method of claim 13 wherein the composition comprises a compound of Formula (VI): 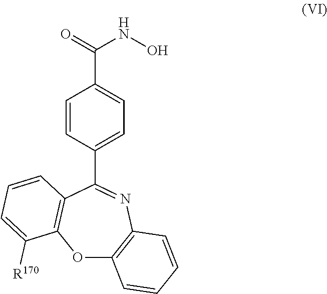
or a pharmaceutically acceptable salt thereof.

18. The method of claim 17 wherein R¹⁷⁰ is selected from: H, halo, —CN, —CF₃, —OCF₃, —C₁-C₆alkyl, and —C₁-C₆alkoxyl.

19. The method of claim 12 wherein the composition comprises a compound selected from:
(Z)-4-(dibenzo[b,f][1,4]oxazepin-11-yl)-N-hydroxybenzamide,
4-(10,11-dihydrodibenzo[b,f][1,4]oxazepin-11-yl)-N-hydroxybenzamide,
N-hydroxy-4-(10-methyl-10,11-dihydrodibenzo[b,f][1,4]oxazepin-11-yl)benzamide,
(Z)-4-(8-chloro-5H-dibenzo[b,e][1,4]diazepin-11-yl)-N-hydroxybenzamide,
(Z)-4-(benzo[b]pyrido[3,2-f][1,4]oxazepin-5-yl)-N-hydroxybenzamide,
(Z)-4-(2-fluorodibenzo[b,f][1,4]oxazepin-11-yl)-N-hydroxybenzamide,
(Z)—N-hydroxy-4-(2-methoxydibenzo[b,f][1,4]oxazepin-11-yl)benzamide,
(Z)-4-(benzo[b]pyrido[4,3-f][1,4]oxazepin-5-yl)-N-hydroxybenzamide,
(Z)-4-(2-(2-(dimethylamino)ethoxy)dibenzo[b,f][1,4]oxazepin-11-yl)-N-hydroxybenzamide,
(Z)—N-hydroxy-4-(8-(trifluoromethyl)dibenzo[b,f][1,4]oxazepin-11-yl)benzamide,
(Z)-4-(dibenzo[b,f][1,4]oxazepin-11-yl)-2-fluoro-N-hydroxybenzamide,
(Z)-5-(4-(hydroxycarbamoyl)phenyl)benzo[b]pyrido[4,3-f][1,4]oxazepine 2-oxide,
(Z)—N-hydroxy-4-(3-methoxydibenzo[b,f][1,4]oxazepin-11-yl)benzamide,
(Z)-3-(dibenzo[b,f][1,4]oxazepin-11-yl)-N-hydroxybenzamide,
(Z)—N-hydroxy-4-(8-methyldibenzo[b,f][1,4]oxazepin-11-yl)benzamide,
(Z)—N-hydroxy-4-(4-methoxydibenzo[b,f][1,4]oxazepin-11-yl)benzamide,
(Z)-4-(9-fluorodibenzo[b,f][1,4]oxazepin-11-yl)-N-hydroxybenzamide,
(Z)—N-hydroxy-4-(7-(trifluoromethyl)dibenzo[b,f][1,4]oxazepin-11-yl)benzamide,
(Z)-4-(7-chlorodibenzo[b,f][1,4]oxazepin-11-yl)-N-hydroxybenzamide,
(Z)-4-(2-chlorodibenzo[b,f][1,4]oxazepin-11-yl)-N-hydroxybenzamide,
(Z)-4-(8-cyanodibenzo[b,f][1,4]oxazepin-11-yl)-N-hydroxybenzamide,
(Z)—N-hydroxy-4-(4-methyldibenzo[b,f][1,4]oxazepin-11-yl)benzamide,
(Z)—N-hydroxy-4-(3-methyldibenzo[b,f][1,4]oxazepin-11-yl)benzamide,
(Z)-4-(benzo[b]thieno[2,3-f][1,4]oxazepin-10-yl)-N-hydroxybenzamide,
(Z)-4-(3-fluorodibenzo[b,f][1,4]oxazepin-11-yl)-N-hydroxybenzamide,
(Z)-4-(8-chlorodibenzo[b,f][1,4]oxazepin-11-yl)-N-hydroxybenzamide,
(Z)—N-hydroxy-4-(3-(trifluoromethyl)dibenzo[b,f][1,4]oxazepin-11-yl)benzamide,
(Z)-4-(6-fluorodibenzo[b,f][1,4]oxazepin-11-yl)-N-hydroxybenzamide,
(Z)-4-(7-cyanodibenzo[b,f][1,4]oxazepin-11-yl)-N-hydroxybenzamide,
(Z)—N-hydroxy-4-(4-hydroxydibenzo[b,f][1,4]oxazepin-11-yl)benzamide,
(Z)—N-hydroxy-4-(1-methoxydibenzo[b,f][1,4]oxazepin-11-yl)benzamide,
(Z)—N-hydroxy-4-(4-(2-methoxyethoxy)dibenzo[b,f][1,4]oxazepin-11-yl)benzamide,
(Z)-4-(1-fluorodibenzo[b,f][1,4]oxazepin-11-yl)-N-hydroxybenzamide,
(Z)—N-hydroxy-4-(2-(trifluoromethyl)benzo[f]pyrido[2,3-b][1,4]oxazepin-6-yl)benzamide,
(Z)-4-(11-cyclopropyl-11H-benzo[b]pyrido[2,3-e][1,4]diazepin-5-yl)-N-hydroxybenzamide,
(Z)-4-(5-cyclopropyl-5H-dibenzo[b,e][1,4]diazepin-11-yl)-N-hydroxybenzamide,
(Z)-4-(5H-dibenzo[b,e][1,4]diazepin-11-yl)-N-hydroxybenzamide,
(Z)—N-hydroxy-4-(4-(2-morpholinoethoxy)dibenzo[b,f][1,4]oxazepin-11-yl)benzamide,
(Z)-4-(benzo[f]pyrido[2,3-b][1,4]oxazepin-6-yl)-N-hydroxybenzamide,
(Z)-4-(2-fluoro-4-methoxydibenzo[b,f][1,4]oxazepin-11-yl)-N-hydroxybenzamide,
(Z)—N-hydroxy-4-(4-(methylthio)dibenzo[b,f][1,4]oxazepin-11-yl)benzamide,
(Z)—N-hydroxy-4-(4-(trifluoromethyl)dibenzo[b,f][1,4]oxazepin-11-yl)benzamide,
(Z)—N-hydroxy-4-(4-(methylsulfinyl)dibenzo[b,f][1,4]oxazepin-11-yl)benzamide,
(Z)-4-(5H-benzo[e]pyrrolo[1,2-a][1,4]diazepin-11-yl)-N-hydroxybenzamide,
(Z)—N-hydroxy-4-(4-(methylsulfonyl)dibenzo[b,f][1,4]oxazepin-11-yl)benzamide,
(E)-4-((dibenzo[b,f][1,4]oxazepin-11-ylamino)methyl)-N-hydroxybenzamide,
(Z)—N-hydroxy-4-(4-methoxy-8-(trifluoromethyl)dibenzo[b,f][1,4]oxazepin-11-yl)benzamide,
(Z)—N-hydroxy-4-(3-morpholinodibenzo[b,f][1,4]oxazepin-11-yl)benzamide,
(Z)—N-hydroxy-4-(4-propyldibenzo[b,f][1,4]oxazepin-11-yl)benzamide,
(Z)—N-hydroxy-4-(4-(trifluoromethoxy)dibenzo[b,f][1,4]oxazepin-11-yl)benzamide,
(Z)—N-hydroxy-4-(6-methyldibenzo[b,f][1,4]oxazepin-11-yl)benzamide,
(E)-4-(dibenzo[b,f][1,4]oxazepin-11-yl)-3-fluoro-N-hydroxybenzamide,
(E)-6-(dibenzo[b,f][1,4]oxazepin-11-yl)-N-hydroxynicotinamide,
(E)-5-(dibenzo[b,f][1,4]oxazepin-11-yl)-N-hydroxyfuran-2-carboxamide,
(E)-5-(dibenzo[b,f][1,4]oxazepin-11-yl)-N-hydroxythiophene-2-carboxamide,
(Z)-4-(5-ethyl-5H-dibenzo[b,e][1,4]diazepin-11-yl)-N-hydroxybenzamide,
(Z)-4-(5-cyclopropyl-5H-dibenzo[b,e][1,4]diazepin-11-yl)-N-hydroxy-N-methylbenzamide,
(Z)—N-hydroxy-4-(5-isopropyl-5H-dibenzo[b,e][1,4]diazepin-11-yl)benzamide,
(E)-4-((5-cyclopropyl-5H-dibenzo[b,e][1,4]diazepin-11-ylamino)methyl)-N-hydroxybenzamide,
(Z)-4-(4-fluorodibenzo[b,f][1,4]oxazepin-11-yl)-N-hydroxybenzamide,
(Z)—N-hydroxy-4-(5-(2-methoxyethyl)-5H-dibenzo[b,e][1,4]diazepin-11-yl)benzamide,
(E)-4-(2-(dibenzo[b,f][1,4]oxazepin-11-ylamino)ethyl)-N-hydroxybenzamide,
(Z)-4-(11-ethyl-11H-benzo[b]pyrido[2,3-e][1,4]diazepin-5-yl)-N-hydroxybenzamide,
(Z)-4-(5-cyclopropyl-2-fluoro-5H-dibenzo[b,e][1,4]diazepin-11-yl)-N-hydroxybenzamide,
(Z)—N-hydroxy-4-(11-isopropyl-11H-benzo[b]pyrido[2,3-e][1,4]diazepin-5-yl)benzamide,
(Z)-4-(benzo[f]thieno[2,3-b][1,4]oxazepin-5-yl)-N-hydroxybenzamide,
(Z)-6-(4-(dibenzo[b,f][1,4]oxazepin-11-yl)benzamidooxy)-3,4,5-trihydroxytetrahydro-2H-pyran-2-carboxylic acid,
(Z)—N-hydroxy-4-(11-(3-morpholinopropyl)-11H-benzo[b]pyrido[2,3-e][1,4]diazepin-5-yl)benzamide,
(Z)—N-hydroxy-4-(11-(2-morpholinoethyl)-11H-benzo[b]pyrido[2,3-e][1,4]diazepin-5-yl)benzamide,
(Z)-4-(11-(cyclopropylmethyl)-11H-benzo[b]pyrido[2,3-e][1,4]diazepin-5-yl)-N-hydroxybenzamide,
(Z)—N-hydroxy-4-(5-(2-morpholinoethyl)-5H-dibenzo[b,e][1,4]diazepin-11-yl)benzamide,
or a pharmaceutically acceptable salt thereof.

20. The method of claim 12 wherein the composition comprises (Z)-4-(dibenzo[b,f][1,4]oxazepin-11-yl)-N-hydroxybenzamide or a pharmaceutically acceptable salt thereof.

21. The method of claim 1 or 12 wherein the patient harbors a mutant allele of the progranulin gene.

22. The method of claim 21, wherein the mutant allele of the progranulin gene is a mutant T allele of rs5848.

23. The method of any of claim 1 or 12 wherein the compound is administered to a human patient at a daily oral dose of 10 mg-1 gm.

24. The method of claim 23 wherein the compound is administered at a daily oral dose of 20-800 mg.

25. The method of claim 23 wherein the compound is administered at a daily oral dose of 40-600 mg.

26. The method of claim 23 wherein the compound is administered at a daily oral dose of 50-400 mg.

27. A method for targeted treatment of FrontoTemporal Lobar Degeneration (FTLD) in a subject, wherein said method comprises administering an FTLD targeted agent to a subject identified as suffering from FTLD, such that the FTLD is treated in the subject.

28. The method of claim 23, wherein the subject identified as suffering from FTLD is identified by an FTLD diagnostic assay.

29. The method of claim 23, further comprising the step of identifying the subject suffering from FTLD by administering to the subject an FTLD diagnostic assay.

30. The method of claim 23, wherein the FTLD diagnostic assay is an assay that identifies a mutant allele of the progranulin gene.

31. The method of claim 26, wherein the mutant allele of the progranulin gene is a mutant T allele of rs5848.

32. The method of claim 23, wherein the FTLD diagnostic assay is an assay that measures progranulin levels.

33. The method of claim 23, wherein the FTLD diagnostic assay is an assay that measures progranulin mRNA.

34-35. (canceled)

36. A method for treating frontotemporal lobe dementia in a subject, wherein said method comprises administering an FTLD targeted agent to a subject identified as suffering from FTLD, such that the frontotemporal lobe dementia is treated in the subject.

37. The method of claim 36, wherein the subject identified as suffering from FTLD is identified by an FTLD diagnostic assay

38. The method of claim 36, further comprising the step of identifying the subject suffering from FTLD by administering to the subject an FTLD diagnostic assay.

39. The method of claim 36, wherein the FTLD diagnostic assay is an assay that identifies a mutant allele of the progranulin gene.

40. The method of claim 39, wherein the mutant allele of the progranulin gene is a mutant T allele of rs5848.

41. The method of claim 23, wherein the FTLD diagnostic assay is an assay that measures progranulin levels.

42. The method of claim 36, wherein the FTLD diagnostic assay is an assay that measures progranulin mRNA.

43-44. (canceled)
